(19) United States
(12) Patent Application Publication
(10) Pub. No.: US 2023/0313299 A1
(43) Pub. Date: Oct. 5, 2023

(54) METHOD FOR DIAGNOSIS AND/OR PROGNOSIS OF LIVER DISEASE PROGRESSION AND RISK OF HEPATOCELLULAR CARCINOMA AND DISCOVERY OF THERAPEUTIC COMPOUNDS AND TARGETS TO TREAT LIVER DISEASE AND CANCER

(71) Applicants: UNIVERSITÉ DE STRASBOURG, Strasbourg (FR); INSTITUT NATIONAL DE LA SANTE ET DE LA RECHERCHE MEDICALE (INSERM), Paris (FR)

(72) Inventors: Thomas BAUMERT, Freidburg (DE); Joachim LUPBERGER, Strasbourg (FR); Frank Sven JÜHLING, Kehl (DE); Yujin HOSHIDA, Dallas, TX (US)

(21) Appl. No.: 17/905,285

(22) PCT Filed: Mar. 2, 2021
(86) PCT No.: PCT/EP2021/055203

§ 371 Date: Aug. 30, 2022

Related U.S. Application Data
(60) Provisional application No. 62/983,965, filed on Mar. 2, 2020.

(51) Int. Cl. C12Q 1/6883; C12Q 1/6886; C12N 5/071; G01N 33/50

(52) U.S. Cl. CPC C12Q 1/6883; C12Q 1/6886; C12N 5/067; G01N 33/5067; C12Q 2600/158; C12Q 2600/118; C12Q 2600/136; C12N 2506/30

(57) ABSTRACT

The present invention relates to diagnosis and/or prognosis of liver disease progression and risk of hepatocellular carcinoma and the discovery of therapeutic compounds and targets to treat liver disease and cancer.

### FIELD OF THE INVENTION

The present invention relates to diagnosis and/or prognosis of liver disease progression and risk of hepatocellular carcinoma and the discovery of therapeutic compounds and targets to treat liver disease and cancer.

### BACKGROUND OF THE INVENTION

Hepatocellular carcinoma (HCC) is the most common primary malignancy of the liver with rising incidence[1]. HCC usually arises in advanced liver disease of viral and metabolic origin. Chronic hepatitis C (CHC) is a major cause of HCC. The HCC risk still remains elevated post-cure especially in patients with advanced fibrosis [2]. Non-alcoholic steatohepatitis (NASH) patients are also at high risk of developing HCC. Given the change in lifestyle with increasing obesity and diabetes, NASH will replace viral hepatitis as major cause for HCC [3]. Liver fibrosis is the key risk factor of HCC and HCC almost arises in advanced liver fibrosis [4]. There are no approved therapeutic strategies to treat liver fibrosis and prevent liver disease progression to HCC [4]. Due to the high HCC mortality and unsatisfactory treatment options, the development of strategies to treat liver fibrosis and prevent liver disease progression to HCC is a key unmet medical need[4].

Epigenetic regulation is a major determinant of gene expression. Alteration of the epigenetic program plays a key role for pathogenesis of human disease and cancer[5]. Several studies have investigated the role of epigenetics in HCC, however the role of epigenome modifications for liver disease progression and hepatocarcinogenesis is only recently emerging. The inventors and others have recently demonstrated that CHC results in genome-wide epigenetic modifications, which are associated with HCC risk and persist post cure with DAA[6, 7].

The reversibility of epigenetic changes offers a therapeutic perspective to counteract the associated transcriptional changes and their functional consequences for disease biology. Small molecule inhibitors targeting chromatin modifiers or readers are currently explored as therapeutic approaches with a particular focus on cancer[8, 9].

Thus, there is a pressing need of new therapy to treat liver disease and prevent liver disease progression and the development of hepatocellular carcinoma. To identify patients at risk for liver disease progression and HCC, it is important to identify epigenetic and transcriptional changes associated with HCC in CHC and NASH patient and assess their impact as biomarker for surveillance and treatment approaches. The identification of pathways associated with liver disease progression and hepatocarcinogenesis provides opportunities to treat advanced liver disease and prevent HCC.

### SUMMARY OF THE INVENTION

The present invention features, at least in part, a method of diagnosis and/or prognosis of liver disease progression and risk of hepatocellular carcinoma in a subject comprising detection of an epigenetic or transcriptomic change in subjects with liver disease, the method comprising comparing the level of expression of a marker or a plurality of markers in a subject sample; and the level of expression of the marker or plurality of markers in a control sample, wherein the marker or plurality of markers are selected from the group consisting of the markers listed in table S3 and a significant difference between the level of expression of the marker or plurality of markers in the subject sample and the control sample is an indication that the subject is at risk for progression of liver disease and developing a hepatocellular carcinoma. In one embodiment, the subject is at risk for progression of liver disease, death and developing a hepatocellular carcinoma and the liver disease is a non-alcoholic or alcoholic steatohepatitis or chronic hepatitis A, B, C, D, E-related liver disease or liver fibrosis. In another embodiment, the liver disease is a non-alcoholic or alcoholic steatohepatitis or chronic hepatitis B or C-related liver disease or liver fibrosis. In another embodiment, the subject is a direct-acting antivirals-cured patient or a patient cured of viral infection by any treatment.

In one embodiment the marker or plurality of markers have increased expression relative to a control. In another embodiment the marker or plurality of markers have decreased expression relative to a control. In another embodiment, at least one marker has increased expression and at least one marker has decreased expression relative to a control. The marker can be detected in liver tissue, the serum or plasma or urine or any other body part.

In one embodiment, the subject has undergone tumor resection and in another embodiment the subject sample is obtained from non-tumorous liver tissue or tissue surrounding a resected tumor. In another embodiment, the patient has undergone liver biopsy of fine needle aspiration or obtained a blood test. In yet another embodiment the subject sample is selected from the group consisting of fresh tissue, fresh frozen tissue, fixed embedded tissue or subject-derived spheroids. In still another embodiment, the subject-derived spheroids were generated from fresh liver tissue and cultured in spheroid culture medium. In another embodiment, the subject sample is serum or plasma or urine.

The present invention also features a method of diagnosis and/or prognosis of liver disease progression, survival or risk hepatocellular carcinoma in a subject comprising detecting a biomarker selected from the list of 1693 genes displayed in Table S3.

The present invention also features a method of diagnosis and/or prognosis of liver disease progression, survival or risk hepatocellular carcinoma in a subject comprising detecting a biomarker selected from GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR, and GALK1 genes.

The present invention further features a method of assessing the efficacy of a liver disease and hepatocellular carcinoma prevention and treatment approach in a subject with liver disease, the method comprising comparing the level of expression of a marker or a plurality of markers in a subject sample; and the level of expression of the marker or plurality of markers in a second subject sample following the treatment with the therapy, wherein the marker or plurality of markers are selected from the group consisting of the markers listed in Table S3, and a significant difference between the level of expression of the marker or plurality of markers indicates the efficacy of the liver disease or hepatocellular carcinoma prevention therapy.

The present invention further features a method of identifying an agent or compound for use in treatment of viral and metabolic liver disease and chemoprevention or treatment of metabolic and viral hepatocellular carcinoma, said method comprising the steps of providing a sample, contacting the sample with a candidate compound; and detecting an increase or decrease in expression of a marker or a plurality of markers selected from the group consisting of the markers in Table S3, relative to a control, wherein an agent or compound that increases or decreases the expression of said marker or plurality of markers relative to the control is an agent or compound for use in treatment of liver disease or chemoprevention or treatment of metabolic and viral hepatocellular carcinoma. In one embodiment the sample is a subject-derived HCC or adjacent liver tissues or a cancer cell line. In another embodiment the liver cancer cell line is selected from the group consisting of the Huh6, Huh7, Huh7.5.1, Hep3B.1-7, HepG2, SkHep1, C3A, PLC/PRF/5 and SNU-398 cell lines or optionally a combination with another cell line such as LX2 cells, THP1 cells or another cell line or primary cells such as human Kupffer cells or human myofibroblasts or liver sinusoidal endothelial cells.

The sample to be assessed can be any sample that contains a gene expression product. Suitable sources of gene expression products, i.e., samples, can include cells, lysed cells, cellular material for determining gene expression, or material containing gene expression products. Examples of such samples are blood, plasma, lymph, urine, tissue, mucus, sputum, saliva or other cell samples. Methods of obtaining such samples are known in the art. In one embodiment, the sample is derived from an individual who has been clinically diagnosed as having or at risk of developing a hepatic disorder (e.g., liver disease due to any origin/etiology or hepatocellular carcinoma and/or liver fibrosis and cirrhosis). As used herein “obtaining” means acquiring a sample, either by directly procuring a sample from a subject or a sample (tissue biopsy, serum, plasma, primary cell, cultured cells), or by receiving the sample from one or more people who procured the sample from the subject or sample.

Furthermore, the present invention features a screening method for identifying an agent for prevention and treatment of liver disease and hepatocellular carcinoma said method comprising steps of (i) generating different cellular models for liver disease and hepatocellular carcinoma development using the exposure of hepatocyte-like or hepatoma cells to different liver disease injury or hepatocarcinogenic agent, said cellular models exhibiting a Prognostic Epigenetic Signature (PES) poor prognosis—HCC high-risk gene signature, (ii) selection of a candidate compound, (iii) determining the effect of the candidate compound on the PES poor prognosis-high-risk gene signature, (iv) identifying the candidate compound as an agent useful for the treatment and prevention of liver disease and HCC if the candidate compound transforms the PES high-risk gene/poor prognosis signature of the liver cells to a PES HCC low-risk/good prognosis signature in a cellular model system modeling liver injury by hepatocarcinogenic agents. In a first specific embodiment the high-risk gene signature is the poor prognosis/HCC high-risk 1693-gene signature presented in Table S3 or the 25 gene subset thereof presented in Table S4, and wherein the candidate compound is identified as an agent useful for the prevention and treatment of liver disease and HCC if the candidate compound suppresses the expression of the 10 HCC high-risk/poor prognosis genes, or of a subset thereof and/or induces the expression of the 15 HCC low-risk genes/good prognosis genes, or of a subset thereof. In a second specific embodiment the exposure of hepatocyte-like or hepatoma cells to different hepatocarcinogenic agents comprises exposure to the free fatty acids modeling metabolic liver disease such as non-alcoholic liver disease (NASH) and NASH associated fibrosis. In a specific embodiment, the hepatocyte-like cells are co cultured with non-parenchymal liver cells. In a third embodiment, the compound is pre-selected by in vitro, in silico or cell culture drug screening.

The present invention features, in another part, a compound transforming a PES HCC high-risk gene/poor prognosis signature of the liver cells to a PES HCC low-risk/good prognosis signature in a cellular model for liver disease progression and HCC risk for use in the treatment or prevention of liver disease and hepatocellular carcinoma. In a specific embodiment, the PES signature is the 25-gene signature presented in Table S4, and wherein the candidate compound is identified as an agent useful in the treatment or prevention of liver disease and hepatocellular carcinoma if the candidate compound suppresses the expression of the high-risk/poor prognosis genes, or of a subset thereof and/or induces the expression of the low-risk/good prognosis genes, or of a subset thereof. In another specific embodiment the chromatin modifier, regulator or reader-targeting compound is selected from the group consisting of BRD4 inhibitors, BRPF1B inhibitors, G9a/GLP inhibitors, PCAF/GCN5 inhibitors, LSD1 inhibitors, SPIN1 inhibitors, CREBBP/EP300 inhibitors, SMYD2 inhibitors, PRDM9 inhibitors, SMARCA2/4 inhibitors, EZH2 inhibitors, BAZ2A/2B inhibitors, SUV420H1/H2 inhibitors, CECR2/BPTF inhibitors, L3MBTL3 inhibitors, ATAD2A/B inhibitors and PRMT4/6 inhibitors.

The present invention features, in another part, a chromatin modifier, regulator or reader inhibitor or compound targeting epigenetic gene expression regulation for use in the treatment or prevention of liver disease and hepatocellular carcinoma in a subject in need thereof. In a specific embodiment, the subject has a liver disease, comprising chronic hepatitis due to viral infection or metabolic causes such as non-alcoholic steatohepatitis or alcoholic liver disease.

### DETAILED DESCRIPTION OF THE INVENTION

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “liver disease” and/or a related phrase refers to conditions related to the liver, such as alcoholic cirrhosis, alpha-1 antitypsin deficiency, autoimmune cirrhosis, cryptogenic cirrhosis, fulminant hepatitis, hepatitis A, B, C, D and E, and non-alcoholic and alcoholic steatohepatitis, biliary tract disorders, cystic fibrosis, primary biliary cirrhosis, sclerosing cholangitis, biliary obstruction, and cancer (e.g., hepatic carcinoma). Other well-known disease can be found in the prior art, e.g., Wiesner, R. H, Current Indications, Contra Indications and Timing for Liver Transplantation (1996), in Transplantation of the Liver, Saunders (publ.); Busuttil, R. W. and Klintmalm, G. B. (eds.) Chapter 6; Klein, A. W., (1998) Partial Hypertension: The Role of Liver Transplantation, Musby (publ.) in Current Surgical Therapy β.sup.th Ed. Cameron, J. (ed) for more specific disclosure relating to relevant hepatic disorders.

The terms “tumor” or “cancer” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell.

The term “hepatocellular cancer” as used herein, is meant to include primary malignancies of the liver.

The terms “hepatocellular carcinoma” and “HCC” are used herein interchangeably. They refer to the most common type of liver cancer, also called malignant hepatoma. HCC can be secondary to infection with hepatitis C virus (HCV), or secondary to hepatitis B virus (HBV) or hepatitis D virus infection, alcoholic liver disease, non-alcoholic fatty liver disease, hereditary hemochromatosis, alpha 1-antitrypsin deficiency, autoimmune hepatitis, some porphyrias, Wilson's disease, aflatoxin exposure, type 2 diabetes, obesity or other etiologies.

As used herein, a “marker” or “biomarker” is a gene or protein which may be altered, wherein said alteration is associated with a disorder of the liver or a subset thereof. The alteration may be in amount, structure, and/or activity in a tissue or cell having a or modelling a hepatic disorder, as compared to its amount, structure, and/or activity, in a normal or healthy tissue or cell (e.g., a control), and is associated with a disease state, such as cancer and/or cirrhosis. For example, a marker of the invention which is associated with liver disease progression or cancer may have altered copy number, expression level, protein level, protein activity, or methylation status, in a cancer/liver tissue or cancer/liver cell as compared to a normal, healthy tissue or cell. Furthermore, a “marker” includes a molecule whose structure is altered, e.g., mutated (contains an allelic variant), e.g., differs from the wild type sequence at the nucleotide or amino acid level, e.g., by substitution, deletion, or addition, when present in a tissue or cell associated with a disease state, such as cancer. Markers identified herein include diagnostic and therapeutic markers. A single marker may be a diagnostic marker, a therapeutic marker, or both a diagnostic and therapeutic marker.

As used herein, the term “therapeutic marker” includes markers, e.g., markers set forth in the Tables, Figures, or Sequence Listing described herein, which are believed to be involved in the development (including maintenance, progression, angiogenesis, and/or metastasis) of hepatic diseases.

The amount of a marker, e.g., expression or copy number of a marker, or protein level of a marker, in a subject or sample is “significantly” higher or lower than that of a control, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject or sample can be considered “significantly” higher or lower than that of a control if the amount is at least about two, and preferably at least about three, four, or five times, higher or lower, respectively, than the normal amount of the marker.

An “overexpression” or “increased expression” of a marker refers to an expression level or copy number in a test sample that is greater than the standard error of the assay employed to assess expression or copy number, and is preferably at least twice, and more preferably three, four, five or ten or more times the expression level or copy number of the marker in a control sample (e.g., sample from a healthy subject not afflicted with cancer), or preferably, the average expression level or copy number of the marker in several control samples.

An “underexpression” or “decreased expression” of a marker refers to an expression level or copy number in a test sample that is lower than the standard error of the assay employed to assess expression or copy number, but is preferably at least twice, and more preferably three, four, five or ten or more times less than the expression level or copy number of the marker in a control sample (e.g., sample from a healthy subject not afflicted with cancer) or, preferably, than the average expression level or copy number of the marker in several control samples.

As used herein, the term “chemoprevention” is the use of drugs or other natural or synthetic agents, which may be biologic or chemical, to try to reduce the risk of, prevent, suppress, reverse, or delay the development, or recurrence of, premalignant lesions and/or cancer more specifically a hepatocellular cancer. It also includes the treatment of a condition such as advanced liver disease or liver fibrosis, predisposing the patient at risk to develop HCC.

A “marker nucleic acid” is a nucleic acid (e.g., DNA, mRNA, cDNA, microRNA) encoded by or corresponding to a marker of the invention. For example, such marker nucleic acid molecules include DNA (e.g., cDNA) comprising the entire or a partial sequence of any of the nucleic acid sequences encoding markers set forth in the Tables, Figures, or Sequence Listing described herein or the complement or hybridizing fragment of such a sequence. The marker nucleic acid molecules also include RNA comprising the entire or a partial sequence of any of the nucleic acid sequences encoding markers set forth in the Tables, Figures, or Sequence Listing or the complement of such a sequence, wherein all thymidine residues are replaced with uridine residues.

As used herein, the terms “cells” refers to cells in various forms, including but not limited to cells retained in tissues, cell clusters, and individually isolated cells. The term “isolated”, when used herein to characterize cells, means cells which, by virtue of their origin or manipulation, are separated from at least some of the components with which they are naturally associated or with which they are associated when initially obtained or prepared. In the context of the invention, liver cancer cells are used to prepare the cellular model system of HCC development and progression. The term “liver cancer cells” refers to cells that have been isolated from a liver tumor or liver cancer sample (e.g., a biopsy sample) or to cells from a liver tumor-derived cell line or from a liver cancer-derived cell line.

As used herein, the term “non-parenchymal cell” refers to any cell that is not a parenchymal cell. In the liver, non-parenchymal cells produce key paracrine factors that influence growth, metabolism, and transport functions in hepatocytes. Nonparenchymal liver cells include Kupffer cells, stellate cells, liver resident macrophages, liver myofibroblasts, fibroblasts, sinusoidal endothelial cells, immune cells (T, B, NK cells and the like), intrahepatic lymphocytes, and biliary cells as well as cell lines modelling non-parenchymal liver cells.

As used herein, the term “gene” refers to a polynucleotide that encodes a discrete macromolecular product, be it a RNA or a protein, and may include regulatory sequences preceding (5′ non-coding sequences) and following (3′ non-coding sequences) the coding sequence. As more than one polynucleotide may encode a discrete product, the term “gene” also includes alleles and polymorphisms of a gene that encode the same product, or a functionally associated (including gain, loss or modulation of function) analog thereof.

The term “gene expression” refers to the process by which RNA and proteins are made from the instructions encoded in genes. Gene expression includes transcription and/or translation of nucleic acid material. The terms “gene expression pattern” and “gene expression profile” are used herein interchangeably. They refer to the expression (i.e., to the level or amount or copy number) of an individual gene or of a set of genes. A gene expression pattern may include information regarding the presence of target transcripts in a sample, and the relative or absolute abundance levels of target transcripts.

As used herein, the term “subject” refers to a human or another mammal (e.g., primate, dog, cat, goat, horse, pig, mouse, rat, rabbit, and the like), that can develop hepatocellular carcinoma, but may or may not be suffering from the disease. Non-human subjects may be transgenic or otherwise modified animals. In many embodiments of the present invention, the subject is a human being. In such embodiments, the subject is often referred to as an “individual” or a “patient” The term “individual” does not denote a particular age, and thus encompasses newborns, children, teenagers, and adults. The term “patient” more specifically refers to an individual suffering from a disease. In the practice of the present invention, a patient will generally be diagnosed with a liver disease.

The term “candidate compound” refers to any naturally occurring or non-naturally occurring molecule, such as a biological macromolecule (e.g., nucleic acid, polypeptide or protein), organic or inorganic molecule, or an extract made from biological materials such as bacteria, plants, fungi, or animal (particularly mammalian, including human) cells or tissues to be tested for an activity of interest. More specifically these candidate compounds are chromatin modifier or chromatin reader inhibitor. In the screening methods of the invention, candidate compounds are evaluated for their ability to modulate the expression of genes of a Prognostic Epigenetic Signature (PES).

The term “small molecule”, as used herein, refers to any natural or synthetic organic or inorganic compound or factor with a low molecular weight. Preferred small molecules have molecular weights of more than 50 Daltons and less than 2,500 Daltons. More preferably, small molecules have molecular weights of less than 600-700 Daltons. Even more preferably, small molecules have molecular weights of less than 350 Daltons.

A “pharmaceutical composition” is defined herein as comprising an effective amount of an agent that has been identified by a method of screening of the invention to be useful in the treatment or prevention of cirrhosis/HCC, and at least one pharmaceutically acceptable carrier or excipient.

As used herein, the term “effective amount” refers to any amount of a compound, agent, or composition that is sufficient to fulfil its intended purpose(s), e.g., a desired biological or medicinal response in a cell, tissue, system or subject. For example, in certain embodiments of the present invention, the purpose(s) may be: to prevent the onset of hepatocellular carcinoma, to slow down, alleviate or stop the progression, aggravation or deterioration of the symptoms of liver disease or hepatocellular carcinoma; to bring about amelioration of the symptoms of the disease, or to prevent and cure the disease or hepatocellular carcinoma.

The term “pharmaceutically acceptable carrier or excipient” refers to a carrier medium which does not interfere with the effectiveness of the biological activity of the active ingredient(s) and which is not significantly toxic to the host at the concentration at which it is administered. The term includes solvents, dispersion, media, coatings, antibacterial and antifungal agents, isotonic agents, and adsorption delaying agents, and the like. The use of such media and agents for pharmaceutically active substances is well known in the art (see for example “Remington's Pharmaceutical Sciences”, E. W. Martin, 18th Ed., 1990, Mack Publishing Co.: Easton, PA, which is incorporated herein by reference in its entirety).

The terms “approximately” and “about”, as used in reference to a number, generally include numbers that fall within a range of 10% in either direction of the number (greater than or less than the number) unless otherwise stated or otherwise evident from the context (except where such number would exceed 100% of a possible value).

In eukaryotic cells, DNA is packaged along with histone proteins in a nucleoprotein complex referred to as chromatin. The minimal repeating units of chromatin are the nucleosomes, which enable the folding of chromatin into fibers and higher order structures. Gene regulation on the chromatin level (“epigenetics”) is achieved by nature through dynamic chemical modifications of both DNA and histones and associated proteins or molecules, mediated by specialized “chromatin writer” and “chromatin eraser” or “chromatin regulator” enzymes, collectively referred to as “chromatin modifiers”, “chromatin reader” or “chromatin regulators”.

As used herein, the term “chromatin reader inhibitor” or “chromatin modifier inhibitor” or “chromatin regulator” refers to a chemical compound which modulate the Chromatin reader, modifier or regulator enzyme or associated proteins/molecules by inhibition or enhancement of its function.

The term “Prognostic Gene Signature”, as used herein, refers to molecular biomarkers, gene expression or any other means for identification or prediction of liver disease as fibrosis, cirrhosis progression and/or HCC development that comprises a 1693-gene signature referred as the “Extended Prognostic Epigenetic Signature” (Extended PES or PES Extended), and a subset composed of a 25-gene stromal liver signature predictive of liver disease as HCC development, cirrhosis progression and liver-specific and overall death previously developed by the present inventors. Table S3 provides the identity of the 1693 genes of the signature (PES Extended) and Table S4, which is presented in the Examples section below, provides the identity of the 25 genes of the signature (PES).

As used herein, the term “high-risk genes” refers to genes of the signature whose overexpression correlates with high risk of future liver disease and fibrosis progression, HCC development, and poorer liver-specific and overall survival, and the term “low-risk genes” refers to genes whose overexpression correlates with absence or low risk of future HCC development or liver disease progression and good survival.

The term “liver cells exhibiting a poor prognosis status or HCC high-risk gene signature”, as used herein to characterize liver cells of a cellular model for liver disease as fibrosis, cirrhosis or HCC development and progression according to the invention, refers to cells in which the high-risk genes, or a subset thereof, are overexpressed, and in which the low-risk genes, or a subset thereof, are underexpressed. In contrast, the term “liver cells exhibiting a good prognosis status or HCC low-risk gene signature”, as used herein to characterize liver cells (for example hepatocyte-like cells obtained by differentiation according to the invention), refers to cells in which the poor prognosis or HCC high-risk genes of Table S3, or a subset thereof as disclosed in Table S4, are underexpressed or unchanged and in which the PES good prognosis or HCC low-risk genes of Table S4, or a subset thereof, are overexpressed or unchanged.

The present invention pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining the amount, structure, and/or activity of polypeptides or nucleic acids corresponding to one or more markers of the invention, in order to determine whether an individual, eventually with liver disease, is at risk of developing a liver cancer. Such assays can be used for prognostic or predictive purposes to thereby prophylactically treat an individual prior to the onset of a cancer.

Yet another aspect of the invention pertains to monitoring the influence of agents (e. g., drugs or other compounds), administered either to prevent a liver cancer, on the amount, structure, and/or activity of a marker of the invention in clinical trials.

Methods of evaluating the copy number of a particular marker or chromosomal region are well known to those of skill in the art. The presence or absence of chromosomal gain or loss can be evaluated simply by a determination of copy number of the regions or markers identified herein.

Methods for evaluating copy number of encoding nucleic acid in a sample include, but are not limited to, hybridization-based assays. For example, one method for evaluating the copy number of encoding nucleic acid in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal mRNA (e.g., a non-amplified portion of the same or related cell, tissue, organ, etc.) provides an estimate of the relative copy number of the target nucleic acid.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al, ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

In another embodiment mRNA expression is measured using a nCounter Nanostring assay.

The activity or level of a marker protein can also be detected and/or quantified by detecting or quantifying the expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express a marker of the present invention.

Marker and Plurality of Markers

The marker for use in the methods, kits and use according to the invention is preferably a gene selected from the group consisting of the genes listed in Table S3.

The marker may be a HCC high-risk gene (also called poor prognosis gene) or a HCC low-risk gene (also called good prognosis gene).

The HCC high-risk gene is preferably selected from the group consisting of GPRIN3, COL1A2, SLC7A6, CHST11, LBH, TRPC1, IGF2BP2, ARRDC2, SELM, and TMED3.

A HCC low-risk gene is preferably selected from the group consisting of GSTA1, GRB14, SERPINA5, CAT, SLC25A1, PKLR, ADH4, GLYAT, TTR, HPX, RARRES2, ACADSB, CFHR5, DCXR and GALK1.

The marker is preferably a HCC high-risk gene as defined above or a HCC low-risk gene.

When a plurality of markers is used, the markers of said plurality of markers are as defined above.

The plurality of markers as defined above may comprise at least 10 genes, preferably at least 15 genes, more preferably at least 20 genes, still more preferably at least 25 genes and/or at most 180 genes, preferably at most 150 genes, more preferably at most 100 genes, still more preferably at most 50 genes.

The plurality of markers as defined above preferably comprises at least one gene, preferably at least 4 genes, more preferably at least 8 genes, selected from the group consisting GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

In another preferred embodiment, the plurality of markers as defined above comprises GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1 genes. This combination of PES genes advantageously allows a similar or improved capability to identify patients with higher HCC risk compared to the full signature of 1693 genes.

Assessing the Level of Expression of a Marker

The level of expression of a marker as defined above or of the markers of the plurality of markers as defined above may be assessed by any method well known by the skilled person.

The level of expression of a given marker can for example be assessed by quantifying the expressed protein encoded by said marker or by determining the copy number or level of mRNA translated from the marker.

Quantifying an expressed protein or determining the copy number or level of an mRNA may be assessed by any method well known by the skilled person, for example as defined above.

Kit

The present invention also relates to a kit for the diagnosis and/or prognosis of liver disease progression, survival and/or risk of hepatocellular carcinoma, wherein said kit comprises means for assessing the level of expression of a marker as defined above or of a plurality of markers as defined above.

The kit as defined above preferably comprises means for assessing the level of expression of at least 10 markers, preferably at least 15 markers, more preferably at least 20 markers, still more preferably at least 25 markers as defined above and/or at most 180 markers, preferably at most 150 markers, more preferably at most 100 markers, still more preferably at most 50 markers.

An embodiment of the kit as defined above comprises means for assessing the level of expression of the following markers: GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

The means for assessing the level of expression of a marker or a plurality of markers are well known by the skilled person.

Method of Diagnosis and/or Prognosis of Liver Disease Progression, Survival and/or Risk of Hepatocellular Carcinoma

The present invention thus relates to a method of diagnosis and/or prognosis of liver disease progression, survival and/or risk of hepatocellular carcinoma in a subject comprising detecting an epigenetic or transcriptomic change in subject with liver disease.

Detecting an epigenetic or transcriptomic change is particularly carried out by determining the level of expression of a marker or a plurality of markers in a subject sample and comparing the obtained level of expression with those obtained in a control sample.

The method as defined above thus particularly comprises comparing:
a) the level of expression of a marker or a plurality of markers in a subject sample; and
b) the level of expression of the marker or plurality of markers in a control sample,
   wherein the marker or plurality of markers are selected from the group consisting of the genes listed in table S3 and a significant difference between the level of expression of the marker or at least one marker of the plurality of markers in the subject sample and the control sample is an indication that the subject is at risk for progression of liver disease, low survival and/or developing a hepatocellular carcinoma.

For example, the subject is at risk or increased risk for progression of liver disease, low survival and/or developing a hepatocellular carcinoma when at least one gene of the PES Extended high-risk gene is overexpressed and/or when at least one gene of the PES Extended low-risk gene is underexpressed in the subject sample in comparison to the control sample.

On the contrary, the subject is not at risk or has decreased risk for progression of liver disease, low survival and/or developing a hepatocellular carcinoma when at least one gene of the PES Extended high-risk gene is underexpressed and/or when at least one gene of the PES Extended low-risk gene is overexpressed in the subject sample in comparison to the control sample.

On the contrary, the subject is not at risk or has a decreased risk for progression of liver disease, low survival and/or developing a hepatocellular carcinoma when at least one gene of the PES Extended high-risk gene and/or of the PES Extended low-risk gene expression is similar in the subject sample and the control sample. In this case, HCC low-risk are usually not overexpressed compared to controls but at more similar levels than in a subject at risk for progression of liver disease, low survival and/or developing a hepatocellular carcinoma.

The marker or the plurality of markers are particularly as defined above.

The liver disease is particularly as defined above.

The liver disease is preferably a non-alcoholic or alcoholic steatohepatitis, chronic hepatitis A, B, C, D or E related liver disease or of any other etiology.

The subject may have undergone tumor resection.

The subject may have undergone a liver biopsy, fine needle aspiration or a blood or urine test.

The subject may be a patient without treatment or cured by direct-acting antivirals (DAA) and/or interferon-alfa based treatment or a patient cured of or with controlled viral infection, in particular by any treatment.

The subject sample may be selected from the group consisting of a tissue, patient-derived spheroids, serum, plasma or urine.

The tissue as defined above may be a fresh tissue, fresh frozen tissue or fixed embedded tissue.

The tissue as defined above may be a non-tumorous liver tissue or a tissue surrounding a resected tumor.

Patient-derived spheroids may be generated by culturing fresh liver tissue in a spheroid culture medium.

Any spheroid culture medium well known by the skilled person may be used.

The control sample is preferably a sample from a patient without significant liver disease. The patient has particularly no liver disease and is not at risk of developing a liver disease.

The control sample is a sample of the same nature as the subject sample.

The control sample can also be of a patient with liver disease without risk of disease progression or cancer (e.g., early stage liver disease).

For example, both the control sample and subject sample are a tissue, spheroids, serum, plasma or urine.

Alternatively, the level of expression of the marker or of a marker of the plurality of markers in a control sample corresponds to an average of the level of expression of said marker obtained from several healthy subjects or corresponds to a reference value.

The reference value may be determined in function of an average of the level of expression of said marker obtained from several healthy subjects.

The marker or at least one marker of the plurality of markers may have increased expression in the subject sample relative to the control sample.

The marker or at least one marker of the plurality of markers may have decreased expression in the subject sample relative to the control sample.

Alternatively, at least one marker has increased expression in the subject sample relative to the control sample and at least one marker has decreased expression in the subject sample relative to the control sample.

The plurality of markers as defined above preferably comprises at least 10 genes, preferably at least 15 genes, more preferably 20 genes, still more preferably at least 25 genes.

The plurality of markers as defined above preferably comprises at most 180 genes, preferably at most 150 genes, more preferably at most 100 genes, still more preferably at most 50 genes.

The plurality of markers preferably comprises GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

A significant difference between the level of expression of at least one marker, preferably at least two markers, at least four markers, at least eight markers, at least ten markers or at least twelve markers selected from the group consisting of GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1 in the subject sample and the control sample is an indication that the subject is at risk for progression of liver disease, at risk of developing a hepatocellular carcinoma and/or at risk of poor survival.

In Vitro Use of a Marker or a Plurality of Markers for the Diagnosis and/or Prognosis of Liver Disease Progression, Survival and/or Risk Hepatocellular Carcinoma

The present invention also relates to the in vitro use of a marker or a plurality of markers as defined above or of a kit as defined above, for the diagnosis and/or prognosis of liver disease progression, survival and/or risk hepatocellular carcinoma in a subject.

Said marker is preferably a gene selected from the list of 1693 genes displayed in Table S3.

The marker or the plurality of markers are particularly as defined above.

The present invention particularly relates to the in vitro use of a plurality of markers as defined above or a kit as defined above, for the diagnosis and/or prognosis of liver disease progression, survival and/or risk hepatocellular carcinoma in a subject, wherein the plurality of markers comprises at least 10 genes, preferably at least 15 genes, more preferably 20 genes, still more preferably at least 25 genes, for example selected from the 1693 genes listed in Table S3.

The plurality of markers as defined above preferably comprises at most 180 genes, preferably at most 150 genes, more preferably at most 100 genes, still more preferably at most 50 genes.

The plurality of markers preferably comprises GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

An overexpression of at least one marker, preferably at least two markers, at least four markers, at least eight markers, selected from the group consisting of GPRIN3, COL1A2, SLC7A6, CHST11, LBH, TRPC1, IGF2BP2, ARRDC2, SELM, and TMED3 and/or an underexpression of at least one marker, preferably at least two markers, at least four markers, at least eight markers, at least ten markers or at least twelve markers, selected from the group consisting of GSTA1, GRB14, SERPINA5, CAT, SLC25A1, PKLR, ADH4, GLYAT, TTR HPX, RARRES2, ACADSB, CFHR5, DCXR and GALK1 in a subject sample is associated with a risk for progression of liver disease, a risk of developing a hepatocellular carcinoma and/or at risk of poor survival.

Method of Assessing the Efficacy of a Therapy for Liver Disease and/or Hepatocellular Carcinoma Prevention or Treatment

The present invention also relates to a method of assessing the efficacy or prediction the efficacy of a therapy for liver disease and/or hepatocellular carcinoma prevention or treatment in a subject with liver disease, the method comprising comparing:
a) the level of expression of a marker or a plurality of markers in a subject sample, preferably before treatment with the therapy or at a time t1 during treatment with the therapy; and
b) the level of expression of the marker or plurality of markers in a second subject sample, preferably following the treatment with the therapy or at time t2 later than t1,
   wherein the marker or plurality of markers are selected from the group consisting of the genes listed in tables S3 and a significant difference between the level of expression of the marker or plurality of markers indicates the efficacy of the therapy, in particular the efficacy of the prevention and treatment of liver disease and hepatocellular carcinoma.

The subject is preferably a subject at risk of progression of liver disease, death and/or developing a hepatocellular carcinoma.

By “a subject at risk of death”, it is herein meant a subject with poor survival.

The subject may have been diagnosed of at risk for progression of liver disease, poor survival and/or developing a hepatocellular carcinoma by the method of diagnosis and/or prognosis of liver disease progression, survival and/or risk of hepatocellular carcinoma as defined above.

The liver disease is preferably a non-alcoholic or alcoholic steatohepatitis, chronic hepatitis A, B, C, D or E-related liver disease or liver fibrosis.

A decreased expression of at least one marker, preferably at least two markers, at least four markers, at least eight markers, selected from the group consisting of GPRIN3, COL1A2, SLC7A6, CHST11, LBH, TRPC1, IGF2BP2, ARRDC2, SELM, and TMED3, and/or an increased expression of at least one marker, preferably at least two markers, at least four markers, at least eight markers, at least ten markers or at least twelve markers, selected from the group consisting of GSTA1, GRB14, SERPINA5, CAT, SLC25A1, PKLR, ADH4, GLYAT, TTR HPX, RARRES2, ACADSB, CFHR5, DCXR and GALK1 between the level obtained in step a) and the level obtained in step b) particularly indicates the efficacy of the therapy, in particular of the prevention and treatment of liver disease and hepatocellular carcinoma.

An increased expression or similar expression of at least one marker, preferably at least two markers, at least four markers, at least eight markers, selected from the group consisting of GPRIN3, COL1A2, SLC7A6, CHST11, LBH, TRPC1, IGF2BP2, ARRDC2, SELM, and TMED3, and/or a decreased expression or similar expression of at least one marker, preferably at least two markers, at least four markers, at least eight markers, at least ten markers or at least twelve markers, selected from the group consisting of GSTA1, GRB14, SERPINA5, CAT, SLC25A1, PKLR, ADH4, GLYAT, TTR HPX, RARRES2, ACADSB, CFHR5, DCXR and GALK1 between the level obtained in step a) and the level obtained in step b) particularly indicates a lack of efficacy of the therapy, in particular of the prevention and treatment of liver disease and hepatocellular carcinoma.

In case of a lack of efficacy of the therapy, another therapy may be administered to the subject.

The method as defined above may be carried out several time in course of the therapy.

Method of Identifying a Compound Useful for the Prevention or Treatment of Liver Disease and/or Hepatocellular Carcinoma

The present invention also relates to a method of identifying a compound useful for the prevention or treatment of liver disease and/or hepatocellular carcinoma, said method comprising the steps of:
a) providing a sample;
b) contacting the sample with a candidate compound; and
c) detecting an increase or decrease in expression of a marker or a plurality of markers selected from the group consisting of the genes listed in Table S3, relative to a control, and
d) identifying the compound as useful for the prevention or treatment of liver disease and/or hepatocellular carcinoma if it increases or decreases the expression of said marker or plurality of markers relative to the control.

The marker or plurality of markers and the liver-disease are particularly as defined above.

The sample may be or comprise a subject-derived HCC or adjacent liver tissue, a cancer cell, a liver cell line, cells or a cell line derived from a subject-derived HCC or adjacent liver tissue plasma, serum or urine or a combination of cells and cell lines.

The control in step c) may be (i) the level of expression sample of the marker or plurality of markers in a sample not contacted with the candidate compound or (ii) a reference value, in particular as defined above.

Method for Preventing or Delaying the Progression of a Liver Disease or for Preventing, Delaying the Onset of or Treating Hepatocellular Carcinoma

The present invention also relates to a method for preventing or delaying the progression of a liver disease or for preventing, delaying the onset of or treating hepatocellular carcinoma in a subject comprising:
performing the steps of the method of diagnosis and/or prognosis of liver disease progression and/or risk of hepatocellular carcinoma as defined above, and
administering a treatment to the subject diagnosed as at risk for progression of liver disease and/or developing a hepatocellular carcinoma.

The treatment may for example comprises at least one inhibitor of a chromatin reader or modifier, a regulator or reader-targeting or previously identified candidate compounds for treatment of advanced liver disease and HCC prevention (e.g., Nizatidine).

The inhibitor of a chromatin reader or modifier may for example be an inhibitor of p300 histone acetyltransferase (for example C646 or CTK7A), a bromodomain 3 or 4 inhibitor (for example IBET151 or JQ1), an inhibitor of the MLL/WOR5 complex (for example WFR-0103 or MM-102), an inhibitor of HDAC (Histone deacetyltransferases) (for example SAHA, TSA or TMP150).

The inhibitor of a chromatin reader or modifier or regulator or reader-targeting may for example be selected from the group consisting of in a BRPF1B inhibitors, G9a/GLP inhibitors, PCAF/GCN5 inhibitors, LSD1 inhibitors, SPIN1 inhibitors, CREBBP/EP300 inhibitors, SMYD2 inhibitors, PRDM9 inhibitors, SMARCA2/4 inhibitors, EZH2 inhibitors, BAZ2A/2B inhibitors, SUV420H1/H2 inhibitors, CECR2/BPTF inhibitors, L3MBTL3 inhibitors, ATAD2A/B inhibitors or PRMT4/6 inhibitors.

The present invention features, in another part, a chromatin modifier, regulator or reader inhibitor or compound targeting epigenetic gene expression regulation for use in the treatment or prevention of liver disease and hepatocellular carcinoma in a subject in need thereof. In a specific embodiment the subject has a liver disease, comprising chronic hepatitis due to viral infection or metabolic causes such as non-alcoholic steatohepatitis or alcoholic liver disease.

In the method for generating a cellular model identifying an agent for treatment or prevention of liver disease and hepatocellular carcinoma, hepatocyte-like cells, which were obtained by differentiation of liver cancer cells, are submitted to a liver disease causing or hepatocarcinogenic agent. As used herein, the term “submitting cells to a liver disease of hepatocarcinogenic agent” refers to a process in which cells are exposed to (e.g., contacted with and/or incubated with and/or grown in the presence of) a hepatocarcinogenic agent while being cultured. The exposure or contact is performed under conditions and for a time sufficient for the hepatocarcinogenic agent to induce the desired effect (i.e., to induce a stable HCC high-risk gene/poor prognosis signature in the cells). The hepatocarcinogenic agent may be any suitable hepatocarcinogenic agent, and its mechanism of action is not a limiting factor.

In certain embodiments of the invention, submitting hepatocyte-like cells to a hepatocarcinogenic agent may be: submitting said hepatocyte-like cells to persistent HCV infection. Methods for infecting cells (including liver cells) with HCV are known in the art¹¹. The inventors have found that when cells are differentiated with DMSO for a short period of time (about 7-10 days) and then infected with HCV for a short period of time (about 10 days), the PES/HCC risk signature is efficiently induced.

In other embodiments of the invention, submitting hepatocyte-like cells to a hepatocarcinogenic agent may be: submitting said hepatocyte-like cells to persistent HBV infection. As already mentioned above, to prepare a cellular model for HCC development and progression by HBV infection, the starting cells must be HBV susceptible cells (i.e., must be cells that are intrinsically susceptible to HBV infection or cells that have been genetically engineered to overexpress NTCP). Methods for infecting cells (including liver cells) with HBV are known in the art^{54, 55}.

In yet other embodiments of the invention, submitting hepatocyte-like cells to a hepatocarcinogenic agent may be: submitting said hepatocyte-like cells to ethanol exposure. Ethanol may be used at any suitable concentration and the exposure may be performed for any suitable time.

In yet other embodiments of the invention, submitting hepatocyte-like cells to a hepatocarcinogenic agent may be: submitting said hepatocyte-like cells to free fatty acid exposure. Free fatty acid may be used at any suitable concentration and the exposure may be performed for any suitable time. For example, the cells may be exposed to about 400 μM, about 600 μM, about 800 μM, about 1000 μM or more of oleic acid and/or about 200 μM, about 400 μM or about 600 μM or more of palmitic acid, for at least 1 day less than 5 days, preferably 3 days. Any other saturated fatty acid can be used in the method. Preferably, fresh medium containing ethanol is replenished every day. The Examples section below provides a method for exposing cells to free fatty acid.

During the step where the hepatocyte-like cells are submitted to a hepatocarcinogenic agent, DMSO may be present in the cell culture medium (e.g., at a concentration of between about 0.1% to about 3% DMSO vol:vol in the cell culture medium).

In certain embodiments, step (1) of a method for generating different cellular models for liver disease and hepatocellular carcinoma development and progression using the exposure of hepatocyte-like cells to different hepatocarcinogenic agent according to the invention is performed while the hepatocyte-like cells are co-cultured with non-parenchymal liver cells. It is known in the art that co-culture of hepatocytes with non-parenchymal liver cells better represent both normal in vivo liver physiology and disease states. The present inventors have found that, in addition to further improve the in vitro liver cell model, the presence of non-parenchymal liver cells enhances the induction of the HCC high-risk gene signature/PES poor prognosis status in a cell- and dose-dependent manner. While hepatocytes alone are sufficient for generating the HCC high-risk gene signature by exposure to a hepatocarcinogenic agent, this can be amplified through cross-talk with non-parenchymal cells.

Non-parenchymal liver cells that can be used in the context of the present invention include, but are not limited to Kupffer cells, stellate cells, liver resident macrophages, sinusoidal endothelial cells, immune cells (T, B, NK cells and the like), intrahepatic lymphocytes, fibroblasts and myofibroblasts and biliary cells as well as cell lines modelling non-parenchymal liver cells. In certain embodiments, the non-parenchymal cells co-cultured with the hepatocyte-like cells are of a single cell type (e.g., hepatic stellate cells). In other embodiments, non-parenchymal cells co-cultured with the hepatocyte-like cells are a mixture of different types of non-parenchymal cells (e.g., hepatic stellate cells and sinusoidal endothelial cells or hepatic stellate cells and Kupffer cells).

Generally, hepatocyte-like cells and non-parenchymal liver cells are co-cultured under conditions where they are in physical contact. As used herein, the term “physical contact” has its general meaning. For example, cells are in physical contact with each other when they are in a conformation or arrangement that allows for intercellular exchange of materials and/or information to take place.

The invention comprises the following items:

Item 1: A method of diagnosis and/or prognosis of liver disease progression, survival and/or risk of hepatocellular carcinoma in a subject comprising detecting an epigenetic or transcriptomic change in subject with liver disease, the method comprising comparing
a) the level of expression of a marker or a plurality of markers in a subject sample; and
b) the level of expression of the marker or plurality of markers in a control sample,

wherein the marker or plurality of markers are selected from the group consisting of the genes listed in table S3 and a significant difference between the level of expression of the marker or plurality of markers in the subject sample and the control sample is an indication that the subject is at risk for progression of liver disease, at risk of poor survival and/or at risk of developing a hepatocellular carcinoma.

Item 2: The method of item 1, wherein the liver disease is a non-alcoholic or alcoholic liver disease, a liver disease due to viral hepatitis or liver fibrosis.

Item 3: The method of item 2, wherein the liver disease is a non-alcoholic or alcoholic steatohepatitis, chronic hepatitis A, B, C, D or E related liver disease or liver fibrosis.

Item 4: The method according to any one of items 1 to 3, wherein the subject is a patient cured by direct-acting antivirals (DAA) and/or interferon-alfa based treatment or a patient cured of or with controlled viral infection by any treatment.

Item 5: The method according to any one of items 1 to 4, wherein the marker or at least one marker of the plurality of markers have increased expression in the subject sample relative to the control sample.

Item 6: The method according to any one of items 1 to 4, wherein the marker or at least one marker of the plurality of markers have decreased expression in the subject sample relative to the control sample.

Item 7: The method according to any one of items 1 to 4, wherein at least one marker has increased expression in the subject sample relative to the control sample and at least one marker has decreased expression in the subject sample relative to the control sample.

Item 8: The method according to any one of items 1 to 4, wherein at least one gene of the high-risk gene of Table S3 is overexpressed and/or wherein at least one gene of the low-risk gene of Table S3 is underexpressed, in the subject sample in comparison to the control sample.

Item 9: The method according to any one of items 1 to 8, wherein the subject has undergone tumor resection.

Item 10: The method according to any one of items 1 to 9, wherein the subject sample is obtained from a non-tumorous liver tissue or a tissue surrounding a resected tumor.

Item 11: The method according to any one of items 1 to 10, wherein the subject sample is selected from the group consisting of fresh tissue, fresh frozen tissue, fixed embedded tissue, patient-derived spheroids, serum, plasma or urine.

Item 12: The method according to item 11, wherein the patient-derived spheroids were generated by culturing fresh liver tissue in spheroid culture medium.

Item 13: An in vitro use of a marker or a plurality of markers for the diagnosis and/or prognosis of liver disease progression, survival and/or risk hepatocellular carcinoma in a subject, wherein said marker is a gene selected from the genes displayed in Table S3.

Item 14: The method according to any one of items 1 to 12 or the use according to item 13, wherein the marker is or the plurality of markers are a gene selected from the group consisting of GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

Item 15: A method of assessing the efficacy of a therapy for liver disease and/or hepatocellular carcinoma prevention or treatment in a subject with liver disease, the method comprising comparing:
a) the level of expression of a marker or a plurality of markers in a subject sample; and
b) the level of expression of the marker or plurality of markers in a second subject sample following the treatment with the therapy,

wherein the marker or plurality of markers are selected from the group consisting of the genes listed in tables S3 and a significant difference between the level of expression of the marker or plurality of markers indicates the efficacy of the prevention or treatment of liver disease and/or hepatocellular carcinoma.

Item 16: The method of item 15, wherein (i) the subject is at risk for progression of liver disease, death and/or developing a hepatocellular carcinoma and/or (ii) the liver disease is a non-alcoholic or alcoholic steatohepatitis, chronic hepatitis A, B, C, D or E-related liver disease or liver fibrosis.

Item 17: A method of identifying a compound useful for the prevention or treatment of liver disease and/or hepatocellular carcinoma, said method comprising the steps of:
a) providing a sample;
b) contacting the sample with a candidate compound; and
c) detecting an increase or decrease in expression of a marker or a plurality of markers selected from the group consisting of the genes listed in Table S3, relative to a control, and
d) identifying the compound as useful for the prevention or treatment of liver disease and/or hepatocellular carcinoma if it increases or decreases the expression of said marker or at least a marker of the plurality of markers relative to the control.

Item 18: The method according to item 17, wherein the genes is the subset of 25-genes presented in Table S4, and wherein the candidate compound is identified as an agent useful for agent for treatment of liver disease or prevention and treatment of hepatocellular carcinoma if the candidate compound suppresses the expression of the 10 HCC high-risk genes, or of a subset thereof and/or induces the expression of the 15 HCC low-risk genes, or of a subset thereof.

Item 19: The method according to item 17, wherein the sample is or comprises a subject-derived HCC or adjacent liver tissue, a cancer cell, a liver cell line, a combination of liver and non-liver cell lines including non-parenchymal cells or a cell line derived from a subject-derived HCC or adjacent liver tissue plasma, serum or urine.

Item 20: The method according to any of items 17 to 19, wherein the candidate compound is a chromatin modifier, regulator or reader inhibitor or compound targeting epigenetic gene regulation.

Item 21: The method according to item 20, wherein the chromatin modifier, regulator or reader inhibitor or compound targeting epigenetic gene regulation is selected from the list the group consisting of BRPF1B inhibitors, G9a/GLP inhibitors, PCAF/GCN5 inhibitors, LSD1 inhibitors, SPIN1 inhibitors, CREBBP/EP300 inhibitors, SMYD2 inhibitors, PRDM9 inhibitors, SMARCA2/4 inhibitors, EZH2 inhibitors, BAZ2A/2B inhibitors, SUV420H1/H2 inhibitors, CECR2/BPTF inhibitors, L3MBTL3 inhibitors, ATAD2A/B inhibitors or PRMT4/6 inhibitor.

Item 22: A method for preventing or delaying the progression of a liver disease, delaying the onset of or treating hepatocellular carcinoma in a subject comprising:
performing the steps of the method of diagnosis and/or prognosis of liver disease progression and/or risk of hepatocellular carcinoma according to any one of items 1 to 12 or 14, and
administering a preventive treatment to the subject diagnosed as at risk for progression of liver disease and/or at risk of developing a hepatocellular carcinoma.

Item 23: A kit for the diagnosis and/or prognosis of liver disease progression, survival and/or risk of hepatocellular carcinoma, wherein said kit comprises means for assessing the level of expression of GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

Item 24: A method for generating a cellular model for liver disease or hepatocellular carcinoma (HCC) development and progression, said method comprising steps of:
(a) differentiating liver cancer cell line to obtain hepatocyte-like cells; and
(b) submitting said hepatocyte-like cells to one hepatocarcinogenic/fibrosis causing agent such as hepatitis C virus or free fatty acids to obtain liver cells exhibiting a Prognostic Epigentic Signature (PES) high-risk gene signature

Item 25: The method according to item 24, wherein the liver cancer cell line is selected from the group consisting of the Huh6, Huh7, Huh7.5.1, Hep3B.1-7, HepG2, SkHepI, C3A, PLC/PRF/5 and SNU-398 cell lines or optionally a combination with another cell line such as 5 LX2 cells or THP1 cells or another cell line or liver non-parenchymal cells such as Kupffer cells, or myofibroblasts or liver sinusoidal endothelial cells.

Item 26: Use of a cellular model for liver disease progression and HCC risk according to item 24 or item 25 for identifying an agent for the treatment or prevention of liver disease and HCC.

The invention will be further illustrated by the following figures and examples. However, these examples and figures should not be interpreted in any way as limiting the scope of the present invention.

### BRIEF DESCRIPTION OF THE DRAWINGS

FIG. 1. NASH and CHC patients with advanced liver disease share similar epigenetic and transcriptional changes associated with HCC. (A) RNA-Seq (left panel) and ChIP-Seq (right panel) mapping of NASH- and CHC-induced transcriptomic and H3K27ac modifications from patient-derived liver biopsies and resections. Left panel: Unsupervised clustering of significant 4,790 differentially expressed genes in livers from NASH (n=3) and CHC (n=6) compared to control patients (n=3 and 5, respectively). Right panel: Differential signals in H3K27ac ChIP-Seq peaks for corresponding genes in livers from NASH (n=7) and CHC (n=6) compared to control patients (n=6). (B) Significant H3K27ac modifications correlate (Spearman's rank correlation coefficients and p-values) with gene expression changes in both NASH (top panel) and CHC (bottom panel) patients. Prognostic association of gene expression was determined using Cox score for time to overall death in a cohort of patients as described in the material and methods. (C) HALLMARK pathways significantly enriched for H3K27ac modifications in NASH (n=7) and CHC (n=6) compared to control (n=6) patient samples. (D) Significant H3K27ac changes of the 1,693 genes with corresponding transcriptomic changes in NASH and CHC patients derived from B. (E) Venn diagram showing the overlap of significant epigenetically modified genes (shown in D) with corresponding expression changes in NASH and CHC patients with advanced liver disease derived from the ChIP-Seq and RNA-Seq experiments shown in B.

FIG. 2. Advanced fibrosis and risk for HCC development and PES expression in patients with advanced liver disease. (A) The probabilities of future hepatocarcinogenesis and overall survival according to the presence of the epigenetic dysregulation related to the PES. The dysregulation was significantly associated with future HCC development and mortality in patients with HCV-related early-stage cirrhosis. (B) The prevalence of the presence of the epigenetic dysregulation in patients with NASH. The dysregulation was more frequently observed in patients with advanced fibrosis, one of the well-known HCC risk, compared to those with mild fibrosis. (C) The probabilities of future hepatocarcinogenesis and overall survival according to the presence of dysregulation of a gene subset termed the “prognostic epigenetic signature” (PES). (D) The prevalence of the presence of the epigenetic dysregulation in patients with NASH. The PES, including 25 genes, showed better or similar capability to identify patients with higher HCC risk compared to the full signature. The PES was defined as commonly prognostic genes in both HCV and NASH (FDR<0.25).

FIG. 3 (related to FIG. 1A). Sequencing tag density of H3K27ac enrichment in the NFcB2 gene in liver tissue of control, NASH, CHC and DAA-cured patients with HCC. H3K27ac ChIPmentation-based ChIP-Seq was performed on non-infected (Control 1-6; green), on NASH (NASH 1-7; brown), on CHC (CHC 1-6; red) and on DAA/HCC cured (DAA/HCC 1-6; orange) patient livers shown in FIG. 1A. Blue boxes indicate called H3K27ac-enriched loci. Integrative Genomics Viewer (IGV) was used to illustrate reads on the NFκB2 gene.

FIG. 4 (related to FIG. 2). NASH, CHC and DAA/HCC cured patients with advanced liver disease share epigenetic and transcriptional changes associated with HCC risk. (A) RNA-Seq (left panel) and ChIP-Seq (right panel) mapping of transcriptomic and H3K27ac modifications among NASH-, CHC- and DAA/HCC cured patient-derived liver biopsies and resections. Left panel: Unsupervised clustering of significant 5,786 differentially expressed genes in livers from NASH (n=3), CHC (n=6) and DAA/HCC cured (n=3) patients compared to control patients (n=3, 5 and 3 respectively). Right panel: Differential signals in H3K27ac ChIP-Seq peaks for corresponding genes in livers from NASH (n=7), CHC (n=6) and DAA/HCC cured (n=6) compared to control patients (n=6). (B) Significant H3K27ac modifications correlate (Spearman's rank correlation coefficients and p-values) with gene expression changes in DAA/HCC cured patients. Prognostic association of hepatic gene expression was determined as described for FIG. 1B. (C) Venn diagram showing the overlap of significant epigenetically modified genes with corresponding expression changes in NASH, CHC and DAA/HCC cured patients derived from the ChIP-Seq and RNA-Seq experiments shown in panel A. (D) H3K27ac changes of the 1,256 genes with significant transcriptomic changes in NASH, CHC and DAA/HCC cured patients.

FIG. 5. Development of a diagnostic assays for detection of the PES in patient liver tissues using an FDA-approved nCounter probe and development of a noninvasive blood-based immune-assay to detect secreted PES proteins. (A) A custom-made PES hybridization array, designed and applied by the inventors and using the FDA-approved Nanostring nCounter hybridization technology, enables robust detection of the PES in liver tissues of patients with advanced fibrosis (F3-F4) vs. patients with early fibrosis (F0-F1). Gene set enrichment analysis of gene expression revealed a significant (FDR<0.05) induction of PES genes associated with high cancer risk (PES_high risk genes) and suppression of PES genes associated with a protective effect (PES_low risk genes). NES=normalized enrichment score in the assays. (B) Secreted PES-based proteins can be detected by immuno-assays in blood as shown for catalase (CAT). The PES component catalase (CAT) is associated with low cancer risk is strongly and significantly (adjusted P<0.05) impaired in the blood plasma of mice with metabolic liver disease (FRG-NOD mice fed with choline-deficient high fat diet) compared to mice fed with normal diet. Catalase protein abundance was measured with a scioDiscover antibody microarray (Sciomics, Germany).

FIG. 6. Modelling of PES expression and associated epigenetic modifications in cell-based models for viral and metabolic liver disease. (A) Schematic representation of the experimental setup of the model systems for liver disease model systems. H3K27ac marks were profiled by ChIP-Seq following free fatty acid (FFA) treatment (top panel: day 3) or persistent HCV infection (bottom panel: day 10). (B) H3K27ac data of the 1693 genes with significant transcriptomic changes in patients with NASH and chronic hepatitis C (CHC) derived from FIG. 1B, and corresponding changes in FFA-treated or HCV-infected cells derived from the ChIP-Seq experiment shown in panels A and B, and corresponding transcriptional changes in cell culture. (C) GSEA enrichments of 1693-gene signature and 25-gene signature (PES) gene sets in data shown for cell culture on panel B and D. The global status corresponds to the difference between low risk- and high risk-gene expression. (D) H3K27ac and transcriptomic changes for the subset of 25 (PES) genes which are included in 1693-gene signature as shown on panel B. (E) Gene Set Enrichment Analysis (GSEA) pathway analysis of genes associated with H3K27ac modifications in FFA-treated or HCV-infected compared with Mock or non-infected cells from the ChIP-Seq experiment shown in panels A and B.

FIG. 7. Discovery of compounds for prevention and treatment of liver disease using a cell-based assay modeling the clinical PES. (A) Schematic set-up of the drug discovery model using HCV infection as described in FIG. 7 and Materials and Methods. (B-C) Reversal of the poor prognosis/HCC high risk genes to good prognosis/HCC low risk expression by JQ1 (B) and Nizatidine (C) but not by other compounds. Differentiated Huh7.5.1 cells infected with HCV and expressing the PES poor prognosis status were subjected to treatment with compounds indicated and PES was expression was analyzed as described in Methods. The global status corresponds to the difference between low risk- and high risk-gene expression.

FIG. 8. Reversal of the PES poor prognosis status in vivo by a bromodomain-4 inhibitor translates into improvement of liver disease and cancer prevention (A) Schematic representation of in vivo the proof-of-concept study using a mouse model of DEN and choline-deficient, L-amino acid-defined, high-fat diet (CDAHFD)-induced hepatocarcinogenesis. (B) Transcriptomic changes of genes with significant H3K27ac modifications from livers from patients with NASH and chronic hepatitis C (CHC) as explained in FIG. 1D (overlapping genes) and corresponding changes in vehicle or JQ1-treated DEN/CDAHFD mice. (C) Venn diagram showing epigenetically and transcriptionally altered genes in CHC/NASH patients and in DEN/CDAHFD mice, that were corrected by JQ1 treatment. Genes that harbor epigenetic and transcriptomic changes were identified in CHC/NASH patients and in DEN/CDAHFD mice. Among the changed genes, the inventors analyzed whether JQ1 could revert back their H3K27ac levels and their transcript expression levels. (H) GSEA enrichments of 1693-gene signature and 25-gene signature (PES) gene sets in data shown for JQ1-treated DEN/CDAHFD mice. (D) Improvement of liver disease and prevention of HCC. The global status corresponds to the difference between low risk- and high risk-gene expression. GSEA enrichments of 1693-gene signature and 25-gene signature (PES) gene sets in data shown for JQ1-treatment on panel B. The global status corresponds to the difference between low risk- and high risk-gene expression. (E) JQ1 significantly reduces tumor burden in vivo. While body weights are stable, liver weights as well as the numbers of tumors are significantly (*p<0.05; ***p<0.001; ****p<0.0001, unpaired t-test) reduced in JQ1-treated (n=8) compared with untreated (n=8) mice. Results are expressed as means±SEM. (F) Representative macroscopic photographs of livers (×1.5 magnified), H&E and Sirius red staining of liver sections from vehicle and JQ1-treated mice. Tumor nodules are indicated by an arrowhead and are delimited by dashed lines. (G) JQ1 efficiently reduces liver fibrosis. Fibrosis stage was evaluated through quantitative digital analysis of whole-scanned liver sections (collagen proportional area (CPA)) in JQ1-treated (n=3) compared with JQ1-untreated (n=3) mice. Results are expressed as means±SD.

### BRIEF DESCRIPTION OF THE TABLES

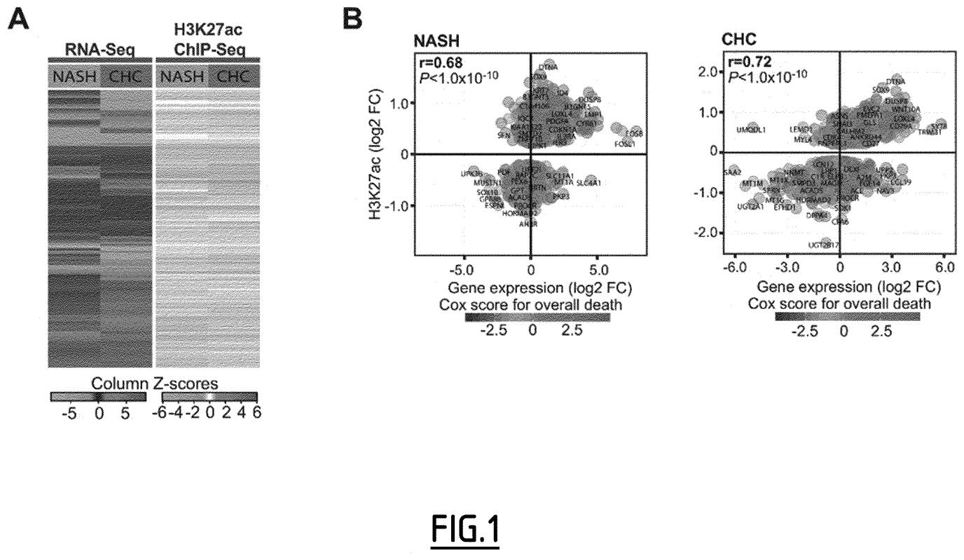

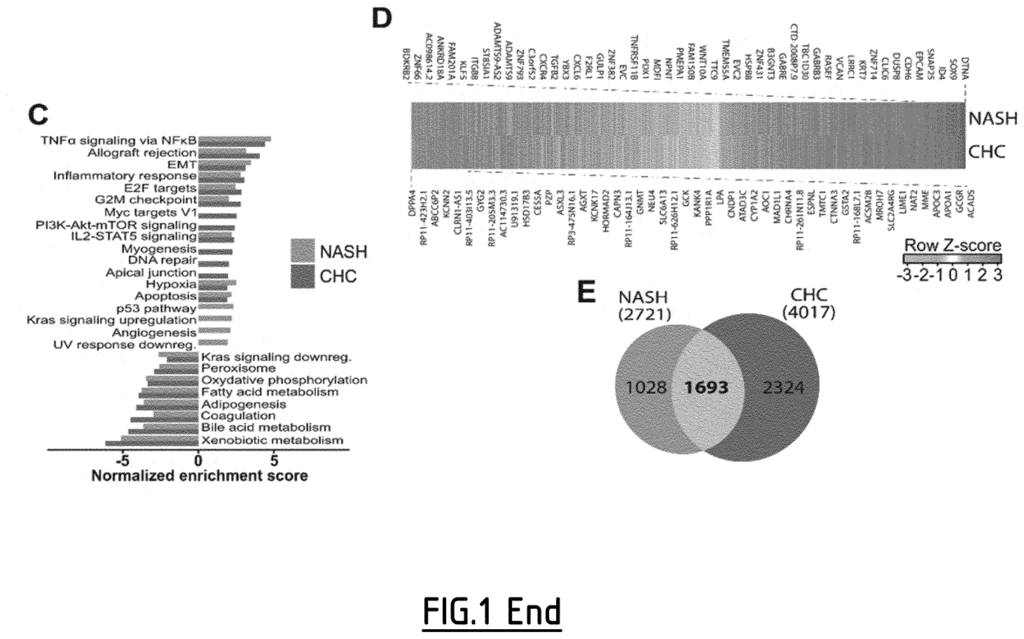

Table S1. Clinical data of patients included in epigenetic analyses using ChIP-seq (related to FIG. 1). Fibrosis was staged according to Kleiner score[51] for NASH and METAVIR score[52] for all the other etiologies. METAVIR score was used for histological grading of the activity of HCV infection[53]. ASV=asunaprevir, CHC=chronic hepatitis C, DCV=daclatasvir, HCC=hepatocellular carcinoma, HCV=hepatitis C virus, LDV=ledipasvir, N/A=not applicable, NASH=non-alcoholic steatohepatitis, RBV=ribavirin, SOF=sofosbuvir.

Table S3 (related to FIG. 1D). List of the 1693 genes of the prognostic epigenetic signature. Epigenetic (H3K27ac) log 2FCs of 1,693 common genes in NASH and CHC patients with similar significant epigenetic modifications and corresponding transcriptional changes.

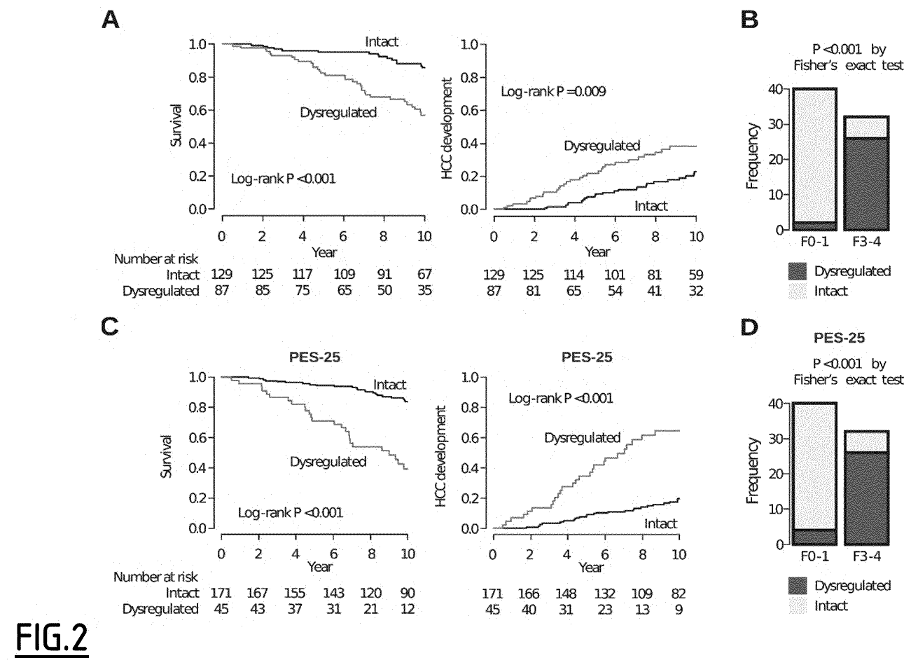

Table S4 (related to FIG. 2C). List of the 25 genes of the prognostic epigenetic signature (PES). List of the 25 genes (high and low-risk genes) with the highest prediction of HCC risk predicted from the 1,693 commonly changed genes on CHC and NASH patients (FDR<0.25) shown in FIG. 2C. The dysregulation was determined by the nearest template prediction [16].

### Examples

### Material and Methods

Human subjects. The inventors analyzed adjacent non-tumorous liver tissue from: 6 control patients without known liver disease and without HCC, 3 patients without known liver disease (F0) and HCC (“spontaneous” HCC), 3 CHC patients without HCC (F3-F4), 3 CHC patients with HCC (F3-F4), 6 DAA-cured CHC patients with HCC (F3-F4), 3 NASH and HCC (F1), 3 NASH and HCC (F4), and 4 NASH without HCC (F4) from patients undergoing surgical liver resection (see Table S1). The protocols were approved by the Ethics Committee of the Strasbourg University Hospitals (DC-2016-2616), Mount Sinai Hospital, New York (HS13-00159), Basel University Hospital (EKNZ 2014-362) and Hiroshima University Hospitals (E-1049-1). Some subjects have been described [6].

ChIPmentation based ChIP-Seq and RNA next-generation sequencing (NGS). ChIPmentation-based ChIP-Seq was performed as described[6, 11]. RNA-Seq was performed as described[6]. Mouse RNA-Seq data was processed as described for patient's data but was instead mapped to the mouse genome mm10 and annotated using the Gencode vM15 gene annotation. Processing of ChIPmentation data was described[6], and data was partially derived from BioProject PRJNA506130. Since late stage fibrosis patients have the highest HCC risk, patient tissues from late stage fibrosis (F3 and F4) samples were included for all H3K27ac analyses as well as from the external CHC RNA-Seq dataset (GSE84346)[12]. Transcriptomic data for NASH patients was derived from external expression dataset (GSE115193)[13] and data for DAA-cured patients was published in BioProject PRJNA506130[6]. The RNA-Seq data from HCV-infected liver cells was published in (GSE126831)[14].

Cell-based models. Huh7.5.1 and human stellate LX2 cells were cultured in Dulbecco's Modified Eagle Medium (DMEM) supplemented with 10% heat-decomplemented fetal bovine serum FBS, gentamycin (0.05 mg/mL) and non-essential amino acids (complete DMEM) at 37° C. with 5% CO2. For proliferation arrest and differentiation (Huh7.5.1thf cells), Huh7.5.1 cells were cultured in complete DMEM supplemented 1% DMSO [5]. All cell lines were certified mycoplasma-free. To analyze the PES induction Huh7.5.1thf cells were infected with HCV Jc1 (genotype 2a/2a) [6] for a total of 10 days. Cells were then treated with Captopril (1 μM), Nizatidine (10 μM), Pioglitazone (1 μM), JQ1 (50 nM). For the FFA model, Huh7.5.1^{dif} cells were cocultured with 20% LX-2 cells. Following co-culture for 3 days in DMEM supplemented with 10% heat-decomplemented FBS, gentamycin and 1% DMSO at 37° C. and 5% CO2, cells were incubated with FFA (800 μM oleic acid and 400 μM palmitic acid) for 72 h [7].

Analysis of gene expression of the prognostic epigenetic signature (PES) using nCounter expression or RNASeq analyses. Profiling of the PES was performed using Nanostring nCounter assay or RNASeq as described[15]. Induction or suppression of the PES in gene expression data was determined as previously reported using the Gene Set Enrichment Analysis (GSEA), implemented in GenePattern genomic analysis toolkits[16-18]. False discovery rate (FDR)<0.05 was regarded as statistically significant. Results are presented as heatmaps showing: (top) the classification of the PES global status as poor (orange) or good (green) prognosis; (bottom) the significance of induction (red) or suppression (blue) of PES poor- or good-prognosis genes. Global status corresponds to the difference between low risk- and high risk-gene enrichments. For PES results are presented as heatmaps showing the significance of induction (red) or suppression (blue) of (top) PES high-risk- or (bottom) low-risk genes.

Protein profiling of mouse plasma samples with liver disease using scioDiscover antibody microarrays (Sciomics, Germany). FGR-NOD mice (n=9) were transplanted with human primary hepatocytes as described. 4 mice were fed with normal diet and 5 mice with choline-deficient high fat diet (Research diet, Brogaarden, Denmark) for 20 weeks. At the end of the diet, the mouse livers were harvested and liver lysates were labelled at an adjusted protein concentration for two hours with scioDye 1 and scioDye 2. After two hours the reaction was stopped and the buffer exchanged to PBS. All labelled protein samples were stored at −20° C. until use. Samples were analysed in a dual-colour approach using a reference based design on 9 scioDiscover antibody microarrays (Sciomics, Germany) targeting 1,360 different proteins with 1,830 antibodies. Each antibody is represented on the array in four replicates. The arrays were blocked with scioBlock (Sciomics, Germany) on a Hybstation 4800 (Tecan, Austria) and afterwards the samples were incubated competitively using a dual-colour approach. After incubation for three hours, the slides were thoroughly washed with 1×PBSTT, rinsed with 0.1×PBS as well as with water and subsequently dried with nitrogen. Slide scanning was conducted using a Powerscanner (Tecan, Austria) with identical instrument laser power and adjusted PMT settings. Spot segmentation was performed with GenePix Pro 6.0 (Molecular Devices, Union City, CA, USA). Acquired raw data were analyzed using the linear models for microarray data (LIMMA) package of R-Bioconductor after uploading the median signal intensities.

Pathway enrichment and correlation analyses. Hallmark pathway enrichment and correlation analyses were performed as described [6].

Statistical analyses. Statistical analyses of NGS data are based on DESeq (RNA-Seq) and MACS2/edgeR (ChIP-Seq) as described [6]. The cell culture and tumorspheroids data are presented as the mean±SD except where mean±SEM. is indicated and were analyzed by the unpaired Student's t-test or the two-tailed Mann-Whitney test as indicated after determination of distribution by the Shapiro-Wilk normality test. The p-values are indicated in the figure legends for each figure panel. P<0.05 was considered significant. Statistical analyzes were performed with GraphPad Prism 6 software. For the antibody capture arrays (sciDiscover, Sciomics, Germany), raw data were normalized using a specialized invariant Lowess method. For analysis of the samples a one-factorial linear model was fitted with LIMMA resulting in a two-sided t-test or F-test based on moderated statistics. All presented p-values were adjusted for multiple testing by controlling the false discovery rate according to Benjamini and Hochberg. Differences in protein abundance between different samples or sample groups are presented as log-fold changes (log FC) calculated for the basis 2. Proteins were defined as differential for log FC>0.5 and an adjusted p value<0.05. In a study comparing samples versus control a log FC=1 means that the sample group had on average a 21=2 fold higher signal as the control group. log FC=−1 stands for 2−1=1/2 of the signal in the sample as compared to the control group (normal diet).

HCC risk profiling. Transcriptome profiles of 72 NASH-affected liver tissues were obtained from NCBI Gene Expression Omnibus database (www.ncbi.nlm.nih.gov/geo, accession number GSE49541). Transcriptomic molecular dysregulation was determined using Nearest Template Prediction (NTP) algorithm as previously described [16] and defined based on p<0.05.

Results

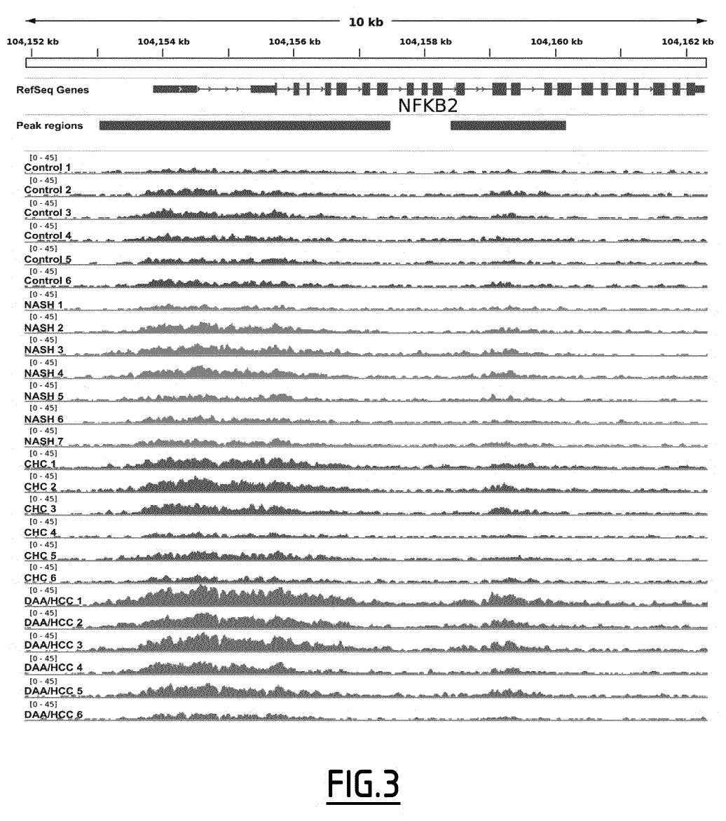

Example 1: NASH and CHC patients with advanced liver disease share similar epigenetic and transcriptional changes associated with HCC risk. To characterize epigenetic and transcriptional modifications in the liver driving HCC risk, the inventors analyzed NASH and CHC liver tissues with advanced liver fibrosis (F3/F4) using ChIPmentation-based ChIP-Seq profiling of the H3K27ac epigenetic marks of active promoters and enhancers combined with RNA-Seq (FIGS. 1A and 3). Within each etiology, epigenetically modified genes significantly correlate with transcriptomic changes (FIG. 1B). Pathways analysis revealed that NASH and CHC patients with advanced liver disease (F3 and F4) display increased H3K27ac levels on genes related to TNFα signaling via NF-κB, inflammatory response, epithelial-to-mesenchymal transition (EMT) and IL2-STAT5, and decreased levels of H3K27ac on genes related to xenobiotic, bile acid, and fatty acid metabolism as well as adipogenesis, coagulation, and oxidative phosphorylation (FIG. 1C). The inventors then identified genes with H3K27ac modifications and concomitant alteration of corresponding transcript expression, and then intersected NASH (n=2,721) and CHC (n=4,017) groups to identify a gene set with modulated expression in both etiologies present in the adjacent tissue of HCC. The inventors uncovered a total of 1,693 genes with common epigenetic and transcriptional changes (FIG. 1D-E, Table S3). Within the genes shared by CHC and NASH, the inventors identified overexpressed oncogenes with increased level of H3K27ac and down-regulated tumor suppressor genes (TSG) with decreased level of H3K27ac. Among the overexpressed oncogenes were FGFR1, a member of the fibroblast growth factor receptor (FGFR) family that plays a key role in the development and progression of HCC[22], the cyclin CCND2, reported to drive tumorigenesis and progression of various cancers including HCC, MLLT3, an oncogene associated to leukemia, CDH11, a cadherin reported to be involved in liver fibrosis and in EMT[23] as well as MAML2, a coactivator of the Notch signaling pathway known to mediate liver carcinogenesis[24]. Downregulated TSG included FANCC, encoding a protein associated to the DNA damage response, and TSC2, a negative regulator of the mTOR signaling pathway[25].

Since the analysis included only a limited number of patients and the large majority having already established HCC without detailed longitudinal data available, the inventors studied the association of the observed gene set modulation with HCC risk and overall mortality in a validation cohort of patients with HCV-related early-stage cirrhosis and longitudinal analysis [26]. Patients with dysregulation of the 1,693 commonly changed genes exhibited a significantly shorter survival and a significantly earlier HCC development than those with transcriptionally intact liver (FIG. 2A). Furthermore, the dysregulation was more prevalent in patients with NASH-related advanced fibrosis defined as F3 or F4, whose HCC risk is higher[27], compared to those with mild fibrosis defined as F0 or F1 (p<0.001) (FIG. 2B). The identified gene set could be condensed to an intersected gene set of 25 genes with highest prediction of HCC risk. This gene set was termed “prognostic epigenetic signature” (PES) (FIG. 2C-D and Table S4). Collectively, this validation analysis suggests that the identified gene expression changes are associated with HCC risk in advanced liver disease.

To assess transcriptional changes that were associated with the presence of advanced fibrosis (F3-4), the inventors intersected liver tissues with and without advanced fibrosis. Using this approach, the inventors found that 43% of the epigenetically modified genes appeared to be linked to the presence of advanced fibrosis (742 of 1,693). Interestingly, the inventors found 27 genes with epigenetic modifications in the adjacent tissue of three patients with spontaneous HCC without fibrosis which were not altered in patients with HCC and fibrosis of any stage and not present in patients without liver disease. These findings suggest that epigenetic modifications associated with hepatocarcinogenesis can occur in the absence of fibrosis.

Next, the inventors studied the role of epigenetic changes for the status of the prognostic liver signature (PLS)—a well characterized stromal liver 186-gene expression signature that has been shown to predict survival and HCC risk in patients with advanced liver disease of all major HCC etiologies[19]. In line with previously published results[6], the inventors observed a modulation of expression of genes predicting HCC high risk in both NASH and CHC patients with advanced liver disease (FIG. 2E, left panel). Interestingly, H3K27ac levels on these 186 genes were correlated with their respective transcript expression (FIG. 2E, right panel, and 2F), suggesting an association between disease-induced epigenetic modifications and the PLS gene expression. Interestingly, the poor-prognosis PLS status remained (FIG. 2E) upon HCV cure in patients with advanced fibrosis and HCC.

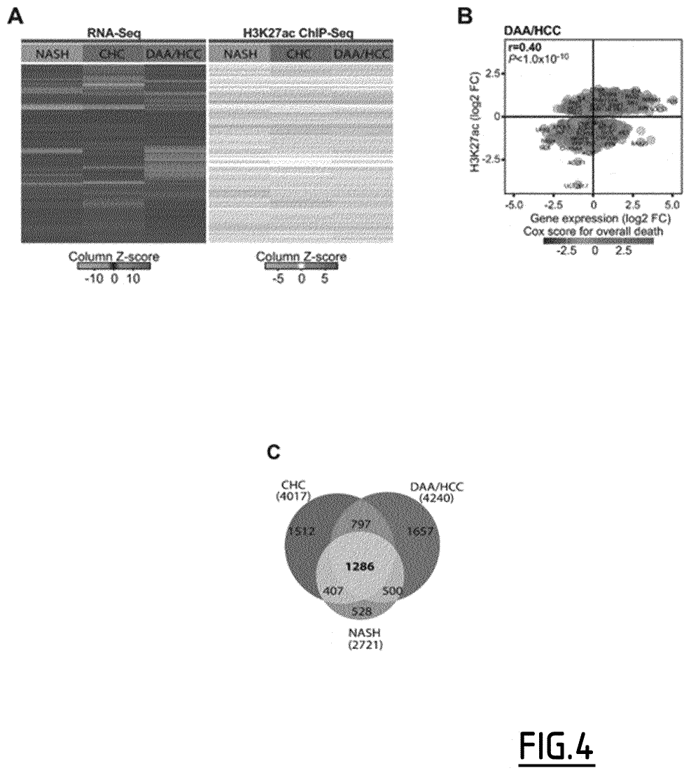

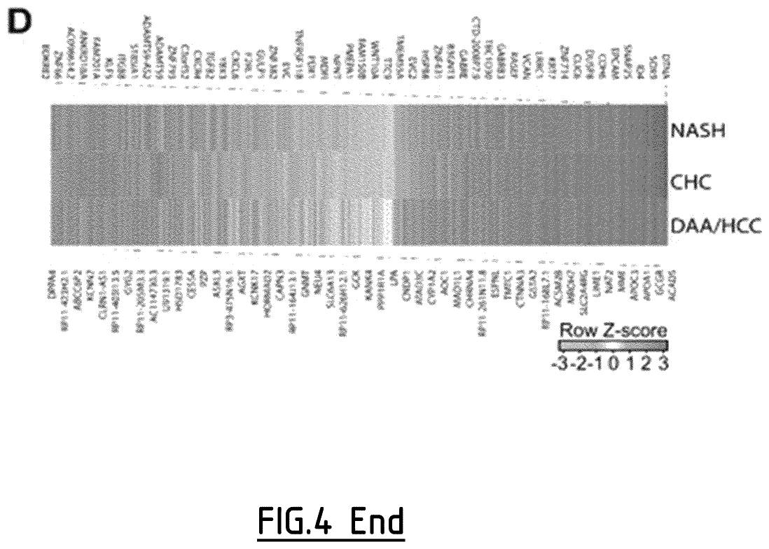

Finally, the inventors intersected the RNA-Seq and H3K27ac ChIP-Seq data among the three patient groups to uncover genes for which changes in their transcript were significantly correlated with H3K27ac modifications, assuming that these genes are most strongly associated with HCC risk. Seventy six percent (1,286 out of 1,693) of the genes identified in both NASH and CHC patient livers were similarly modulated in livers from DAA-cured patients who developed a HCC (FIG. 4A-D).

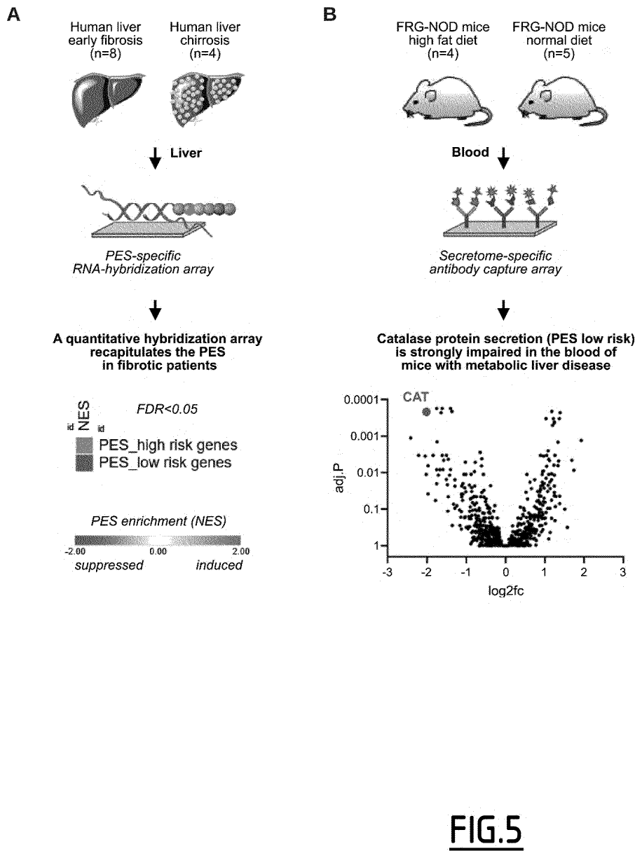

Example 2: Development of liver tissue- and blood-based diagnostic assays for prediction of liver disease progression and HCC risk based on PES gene and protein expression. Based on the patient investigations and discovery of the PES the inventors developed a diagnostic assay for its detection in patient tissues and blood. As shown in FIG. 5, an assay was developed allowing to robustly detect the PES in patient tissues using a the FDA-approved hybridization array nCounter (NanoString). For example in this PES-based assay, the poor prognosis status of the PES was significantly (FDR<0.05) induced in liver tissues of patients with advanced liver disease and cirrhosis compared to livers of patients with mild fibrosis reflecting the evident risk of liver disease progression and HCC risk in cirrhotic patients³⁶. Importantly, PES components are also secreted into the blood and can be detected by antibody capture array (sciDicover, Sciomics, Germany). As an example, the inventors a detected significant decreased expression of the corresponding protein Catalase of the PES poor prognosis gene CAT in the blood of mice with diet-induced metabolic-associated liver disease (FIG. 5B). Collectively, these data demonstrate that the PES can be detected in liver tissue using an FRD-approved technology and secreted proteins corresponding to PES genes can be used to develop a minimal invasive diagnostic assay detecting risk for liver disease progression, based PES and its components.

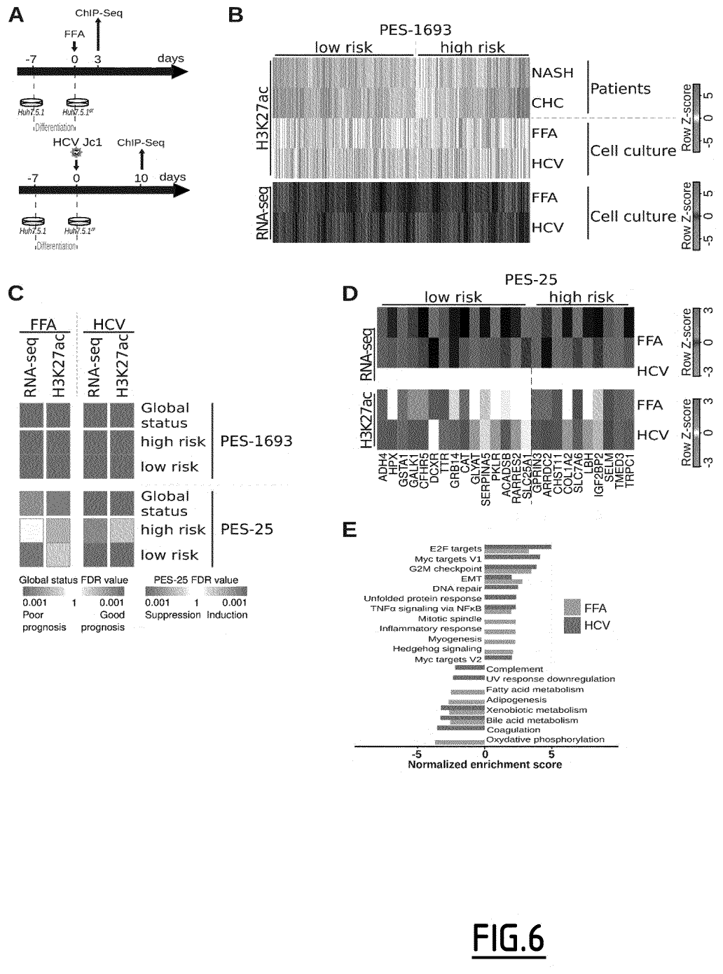

Example 3: Modeling of the patient PES in human liver cell-based systems. The inventors next aimed to model the epigenetic changes observed in patients in cell-based models that partially recapitulates transcriptomic and proteomic changes in patients with chronic liver disease. Dimethyl sulfoxide-differentiated Huh7.5.1 cells (Huh7.5.1 ^{dif} cells) infected by HCV showed transcriptomic and proteomic changes found in liver tissue from HCV-infected patients. Next, the inventors established a cell culture system aiming to model transcriptional changes in metabolic liver injury by using a co-culture of Huh7.5.1^{dif} and LX2 stellate cells treated with free fatty acids (FFA). ChIPmentation-based ChIP-Seq profiling of the H3K27ac epigenetic marks and RNA-Seq analyses (FIG. 6A) revealed that both persistent HCV infection and FFA treatment led to similar epigenetic and transcriptomic changes in cell culture that were observed in patients with NASH and CHC (FIG. 6B). In concordance with the results found in patient-derived liver tissues, GSEA analyses revealed perturbation of pathways mediating TNFα signaling activation, E2F targets, G2M checkpoint, and EMT signaling (FIG. 3E). Importantly, the 1693-gene signature and 25-gene signature (PES) gene sets were induced with poor prognosis/high risk status in the above-mentioned cell culture models under treatment with FFA or infections with HCV (FIG. 6B-D). Collectively, these analyses demonstrate that the cell-based models model the clinical PES and partially recapitulates epigenetic and associated transcriptional alterations that are found in patients with liver disease such as CHC and NASH.

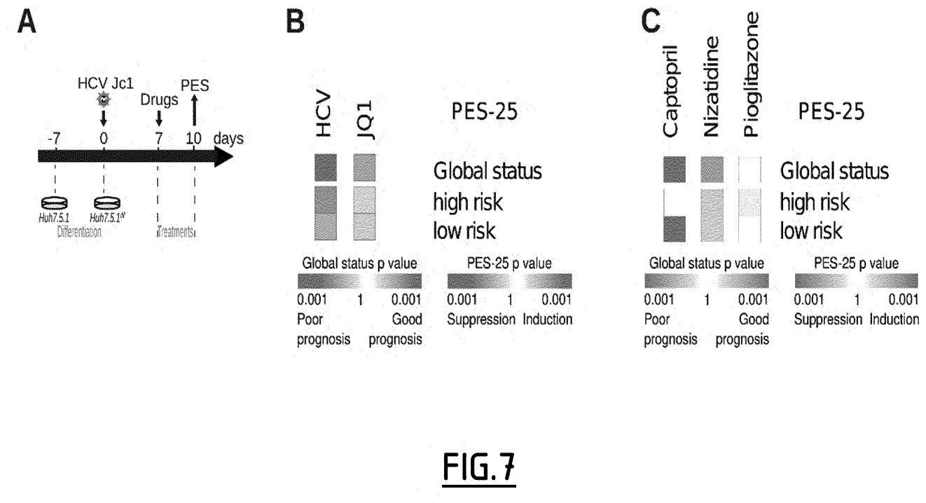

Example 4: Discovery of compounds for prevention and treatment of liver disease and cancer using the PES cell-based model. Aiming to establish a PES-based drug discovery platform, the inventors studied whether the cell-based assays described in Example 3 can be applied to identify compounds which modulate the PES gene expression status from HCC high risk/poor prognosis status to HCC low risk/good prognosis status. As shown in FIG. 7A the inventors first induced the PES poor prognosis/HCC high risk status by infecting differentiated Huh7.5.1 cells with recombinant HCV. The inventors then treated the cells with different small molecules and studied the PES (25 gene subset)expression by RNA-Seq or nCounter Nanostring technology as shown in Example 2. Treatment with bromodomain-4 inhibitor JQ1 reverted the 25-gene signature (PES) (FIG. 7B) from a HCC high risk, poor prognosis status to a HCC low risk, good prognosis as shown by PES (25 gene subset) gene expression analyses. Similar results were obtained when Nizatidine, a HRH2 antagonist was used to incubate the cells (FIG. 7C). These data demonstrate that a cell-based model can identify compounds which revert the high HCC risk, poor prognosis status to HCC low risk, good prognosis status of the PES.

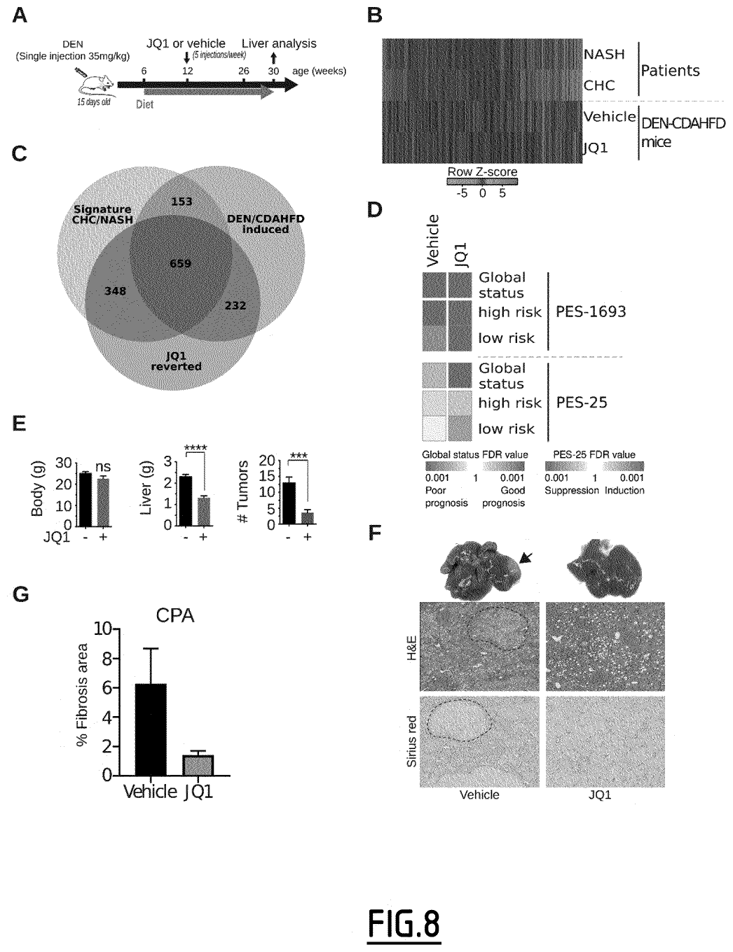

Example 5: Reversal of the PES poor prognosis status in vivo translates into improvement of liver disease, inhibition of fibrosis progession and cancer prevention. To investigate (1) whether the PES is modeled in animal models for liver disease and (2) reversal of the PLS poor prognosis status by a compound correlates with improvement of liver disease and cancer prevention, the inventors studied the 1693 gene signature and the 25 gene subset expression in a NASH-fibrosis-HCC mouse model, in which the mice are treated with the BRD inhibitor JQ1 following development of liver disease. In this model a Choline-deficient high-fat-diet (CDHFD) diet combined with DEN, a chemical carcinogen, induces liver fibrosis progressing to HCC. Genetically, DEN-induced tumors resemble human tumors with poor prognosis38 and DEN administration changes the proportion of heterochromatin and euchromatin, suggesting an alteration of epigenetic marks. Mice were treated with JQ1 after 6 weeks of CDAHFD feeding when liver disease developed (FIG. 8A). Transcriptomic profiling of DEN/CDAHFD mouse livers (FIG. 8B-C) unravelled gene expression changes on a large fraction of the 1693 genes of the 1693-gene signature that were similarly modulated in patients with CHC and NASH suggesting that the DEN/CDAHFD mouse model enables the study of epigenetic/transcriptomic changes associated with human liver carcinogenesis. Furthermore, the inventors' analyses revealed that expression of 59% (1007 out of 1693 genes) of H3K27ac-altered genes in patients were reverted by JQ1 treatment in DEN/CDAHFD mice, and 1693-gene signature and 25-gene signature (PES) gene sets were correspondingly (i.e., reversely) enriched after treatment (FIG. 8B-D). Analyses of liver fibrosis and HCC revealed that both the expression of the 1693 gene signature and the 25 gene subset expression and perturbation by JQ1 was associated with improvement of liver disease, fibrosis and HCC prevention. JQ1 treatment significantly reduced liver weights of DEN/CDAHFD mice without altering their body weight (FIG. 8E). Quantification of tumor nodules revealed an overall decrease in their number irrespective of their size and location (FIG. 8E-F). Moreover, JQ1 significantly (p<0.05) reduced liver fibrosis measured by collagen proportionate area (CPA) (FIG. 8G). Collectively, these data show that (i) fibrotic liver disease progressing to HCC is associated with induction of the PES poor prognosis status and (2) reversal of the 1693 gene signature and the 25 gene subset (PES) poor prognosis to good prognosis status by a small molecule targeting bromodomain-4 in vivo translates into improvement of liver disease and HCC prevention.

### Discussion

HCC prevention in patients with advanced liver fibrosis is likely the most effective strategy to improve patient survival, because tumor recurrence after surgical treatment is frequent, and therapeutic approaches for advanced disease as well as HCC remain unsatisfactory[46, 47]. Approved therapies for NASH are absent and late-stage clinical trials show only moderate success. In CHC, DAA-cured patients with advanced fibrosis remain at risk for HCC[2].

Here, the inventors have shown that liver injury in major viral and metabolic etiologies of liver diseases and HCC (CHC and NASH) results in similar epigenetic footprints and transcriptional changes associated with liver disease progression and HCC risk (FIG. 1-2). This finding is in line with the clinical and pathological observation that CHC and NASH share many phenotypes such as steatosis, insulin resistance, inflammation and fibrosis [43] and that HCCs of CHC and NASH exhibit similar deregulated pathways and genetic footprints[44, 45]. These observations also indicate that HCV infection may serve as a model for understanding progression of liver disease progression and hepatocarcinogenesis in NASH.

Based on these analyses the inventors identified a set of 1693 commonly changed genes that was associated with significantly shorter survival and a significantly earlier HCC development than those with transcriptionally intact liver suggesting that this gene set (1693-gene signature) enables to predict HCC risk and survival. To assess transcriptional changes that were associated with the presence of advanced fibrosis (F3-4), the inventors intersected liver tissues with and without advanced fibrosis. Using this approach, the inventors found that a large fraction of the epigenetically modified genes appeared to be linked to the presence of advanced fibrosis. Furthermore, the more prevalent dysregulation in patients with NASH-related advanced fibrosis defined as F3 or F4, compared to those with mild fibrosis defined as F0 or F1 (FIG. 1) suggests that this gene set may also associated with fibrosis progression and predict progression of fibrotic liver disease.

The signature of 1693 genes as well as condensed intersected gene set of 25 genes with highest prediction of HCC, termed “prognostic epigenetic signature” (PES) may serve a biomarker to predict liver disease progression, HCC risk and survival in patients. Collectively, the inventors' validation analysis in a second cohort suggests the validity of this approach. The detection of the PES in patient liver tissue by a commercially available and FDA approved technology (nCounter, Nanostring) demonstrates that the PES can be used to detect risk of disease progression of fibrotic liver disease and HCC in clinical material (FIG. 2). The detection of a protein corresponding to a PES gene in the blood of an animal model for liver disease (FIG. 2) opens a perspective to develop a minimal or non-invasive blood-based assay to predict liver disease progression, HCC risk and survival in patients with liver disease.

Furthermore, the inventors' data across models demonstrate that inhibition of disease-induced epigenetic changes robustly inhibits gene expression associated with liver disease progression and HCC risk and markedly and significantly inhibits hepatocarcinogenesis in a state-of-the-art in vivo model for NASH-induced HCC (FIG. 5). Collectively, these data demonstrate that epigenetic modifications are a target for treatment of advanced liver disease and HCC prevention. HCC prevention in patients with advanced liver fibrosis is likely the most effective strategy to improve patient survival, because tumor recurrence after surgical treatment is frequent, and therapeutic approaches for advanced disease remain unsatisfactory. Approved therapies for NASH and fibrosis are absent and late-stage clinical trials show only moderate success. In CHC, DAA-cured patients with advanced fibrosis remain at risk for HCC. Addressing this key unmet medical need, the inventors identify BRD4 as a candidate target and BRD4 inhibitor JQ1 as a candidate compound for treatment of liver fibrosis and HCC prevention.

There is an unmet technical need for experimental systems modeling human disease-specific gene expression to understand liver disease biology and hepatocarcinogenesis and enable drug discovery for treatment of advanced liver disease and HCC. Here we addressed this need by the development of a simple and robust liver cell-based system that models gene expression of patients with fibrotic and carcinogenic liver disease caused by HCV and NASH—two major etiologies of advanced liver disease progressing to HCC. Our findings demonstrate the clinical 1693 gene signature and the 25 gene subset (PES) can be experimentally modeled in a cell-based model (cPES). The cPES model offers opportunities to discover compounds for treatment of chronic liver disease treatment and HCC across the distinct liver cancer etiologies, in a fast-track high-throughput screening format as shown for JQ1 or Nizatidine. The innovation of the cPES model is its read-out of a clinically identified cPES predicting disease progression and HCC risk, which enables drug and target discovery for prevention and treatment of liver disease, fibrosis and HCC. The translatability of the compounds identified by the cPES assay is shown by in vivo validation of bromodomain-4 inhibitor JQ1 in a state-of-the-art mouse model for liver disease and hepatocarcinogenesis where reversal of the PES poor prognosis/HCC high risk status (shown for both the 1693 signature and the 25 subset, FIG. 8) by JQ1 was associated with inhibition of liver disease progression, improvement of liver fibrosis and reduced HCC development.

### Tables

TABLE S1
Clinical data of patients included in epigenetic analyses using ChIP-seq (related to FIG. 1).

| | | | Liver | | Viral | METAVIR | Fibrosis | Antiviral | Viral load |
|---|---|---|---|---|---|---|---|---|---|
| Group | Gender | Age | disease | Tumor | genotype | grade | stage | treatment | (log10 IU/ml) |
| Controls | F | 55 | Minimal | No | N/A | N/A | F0 | N/A | N/A |
| | | | hepatitis | | | | | | |
| Controls | M | 46 | Minimal | No | N/A | N/A | F0 | N/A | N/A |
| | | | hepatitis | | | | | | |
| Controls | F | 40 | Lobular | No | N/A | N/A | F0 | N/A | N/A |
| | | | hepatitis | | | | | | |
| Controls | F | 53 | Minimal | No | N/A | N/A | F0 | N/A | N/A |
| | | | hepatitis | | | | | | |
| Controls | M | 56 | Lobular | No | N/A | N/A | F0 | N/A | N/A |
| | | | hepatitis | | | | | | |
| Controls | F | 58 | Minimal | No | N/A | N/A | F0 | N/A | N/A |
| | | | hepatitis | | | | | | |
| HCC w/o | F | 71 | No | HCC | N/A | N/A | F0 | N/A | N/A |
| liver | | | | | | | | | |
| disease | | | | | | | | | |
| HCC w/o | M | 66 | No | HCC | N/A | N/A | F0 | N/A | N/A |
| liver | | | | | | | | | |
| disease | | | | | | | | | |
| HCC w/o | F | 65 | No | HCC | N/A | N/A | F0 | N/A | N/A |
| liver | | | | | | | | | |
| disease | | | | | | | | | |
| NASH | M | 65 | NASH | HCC | N/A | N/A | F1 | N/A | N/A |
| NASH | M | 84 | NASH | HCC | N/A | N/A | F1 | N/A | N/A |
| NASH | M | 78 | NASH | HCC | N/A | N/A | F1 | N/A | N/A |
| NASH | M | 27 | NASH | No | N/A | N/A | F4 | N/A | N/A |
| NASH | M | 63 | NASH | HCC | N/A | N/A | F4 | N/A | N/A |
| NASH | M | 73 | NASH | HCC | N/A | N/A | F4 | N/A | N/A |
| NASH | M | 76 | NASH | HCC | N/A | N/A | F4 | N/A | N/A |
| NASH | F | 65 | NASH | No | N/A | N/A | F4 | N/A | N/A |
| NASH | F | 47 | NASH | No | N/A | N/A | F4 | N/A | N/A |
| NASH | F | 68 | NASH | No | N/A | N/A | F4 | N/A | N/A |
| CHC | M | 52 | CHC | No | 1a | A3 | F3 | Naïve | 5.82 |
| CHC | M | 54 | CHC | HCC | 1b | A1 | F4 | Relapse to | 4.64 |
| | | | | | | | | SOF/DCV/RBV | |
| CHC | M | 68 | CHC | HCC | 2a | A3 | F3 | Naïve | 5.40 |
| CHC | F | 48 | CHC | No | 3a | A3 | F4 | Naïve | 6.06 |
| CHC | M | 65 | CHC | No | 1b | A2 | F4 | Naïve | 6.35 |
| CHC | M | 51 | CHC | HCC | 3a | A2 | F4 | Relapse to | 6.58 |
| | | | | | | | | SOF/RBV | |
| Cured | M | 58 | Cured | HCC | 1a | A0 | F4 | SOF/LDV | Undetectable |
| CHC | | | CHC | | | | | | |
| Cured | F | 79 | Cured | HCC | 1b | A2 | F4 | DCV/ASV | Undetectable |
| CHC | | | CHC | | | | | | |
| Cured | M | 63 | Cured | HCC | 2a | A2 | F4 | SOF/RBV | Undetectable |
| CHC | | | CHC | | | | | | |
| Cured | M | 69 | Cured | HCC | 1b | A2 | F3 | DCV/ASV | Undetectable |
| CHC | | | CHC | | | | | | |
| Cured | M | 73 | Cured | HCC | 1b | A2 | F3 | DCV/ASV | Undetectable |
| CHC | | | CHC | | | | | | |
| Cured | M | 75 | Cured | HCC | 1b | A2 | F3 | SOF/LDV | Undetectable |
| CHC | | | CHC | | | | | | |

Fibrosis was staged according to Kleiner score for NASH and METAVIR score for all the other etiologies. METAVIR score was used for histological grading of the activity of HCV infection.
ASV = asunaprevir, CHC = chronic hepatitis C, DCV = daclatasvir, HCC = hepatocellular carcinoma, HCV = hepatitis C virus, LDV = ledipasvir, N/A = not applicable, NASH = non-alcoholic steatohepatitis, RBV = ribavirin, SOF = sofosbuvir.

TABLE S2

| | | | Liver | | Viral | METAVIR | Fibrosis | Antiviral | Viral load |
|---|---|---|---|---|---|---|---|---|---|
| Group | Gender | Age | disease | Tumor | genotype | Grade | Stage | treatment | (log10 UI/ml) |
| Spheroids | M | 72 | NASH | HCC | N/A | N/A | F4 | N/A | N/A |
| Spheroids | M | 83 | No | HCC | N/A | N/A | F1 | N/A | N/A |
| Spheroids | M | 72 | NASH | HCC | N/A | N/A | F4 | N/A | N/A |
| Spheroids | M | 65 | Cured | HCC | 3 | A1 | F1 | PEG IFN and | Undetectable |
| | | | CHC | | | | | RBV | |
| Spheroids | F | 28 | NAFLD | HCA | N/A | N/A | F0 | N/A | N/A |

TABLE S3
1693 gene-signature also referred as PES Extended. Epigenetic
(H3K27ac) log2FCs of 1693 common genes in NASH and CHC
patients with similar significant epigenetic modifications
and corresponding transcriptional changes. Negative
changes indicate good prognosis genes while positive
changes indicate poor prognosis genes

| Gene | NASH | HCV |
|---|---|---|
| DPPA4 | −0.99149595 | −1.50988763 |
| RP11-423H2.1 | −0.94754738 | −1.48155106 |
| ABCC6P2 | −1.24512898 | −1.44208662 |
| KCNN2 | −0.76184643 | −1.39228038 |
| CLRN1-AS1 | −0.96441909 | −1.3557784 |
| RP11-403I13.5 | −0.85779975 | −1.26410861 |
| GYG2 | −0.69968025 | −1.26305098 |
| RP11-205M3.3 | −0.66576087 | −1.19639311 |
| AC114730.3 | −0.95312175 | −1.15783489 |
| U91319.1 | −0.59765221 | −1.15552389 |
| HSD17B3 | −0.93519793 | −1.15249858 |
| CES5A | −0.58376485 | −1.15154589 |
| PZP | −0.69107133 | −1.14231349 |
| ASXL3 | −0.43301035 | −1.13917046 |
| RP3-475N16.1 | −1.13779469 | −0.98193217 |
| AGXT | −1.12923854 | −0.75283994 |
| KCNK17 | −0.90529918 | −1.124968 |
| HORMAD2 | −1.09406334 | −1.11939868 |
| CAPN3 | −0.85622675 | −1.11518713 |
| RP11-164J13.1 | −0.85622679 | −1.11518708 |
| GNMT | −1.1093108 | −1.01696047 |
| NEU4 | −0.9270435 | −1.10879394 |
| SLC6A13 | −0.90009565 | −1.09119231 |
| RP11-626H12.1 | −0.84179363 | −1.07827663 |
| GCK | −1.06523593 | −0.86809113 |
| KANK4 | −0.74251028 | −1.0564263 |
| PPP1R1A | −0.7017774 | −1.03625106 |
| LPA | −0.49306007 | −1.0169621 |
| CNDP1 | −0.63792646 | −0.9987607 |
| ATAD3C | −0.99811532 | −0.83210312 |
| CYP1A2 | −0.98668091 | −0.81786809 |
| AOC1 | −0.53624729 | −0.98432856 |
| MAD1L1 | −0.97826581 | −0.87221391 |
| CHRNA4 | −0.81746823 | −0.96924072 |
| RP11-261N11.8 | −0.81746808 | −0.96924071 |
| ESPNL | −0.94309271 | −0.70166788 |
| TMTC1 | −0.4180584 | −0.94205862 |
| CTNNA3 | −0.42221582 | −0.94007736 |
| GSTA2 | −0.93792451 | −0.88896097 |
| RP11-168L7.1 | −0.48701285 | −0.93286311 |
| ACSM2B | −0.91701906 | −0.89974648 |
| MROH7 | −0.89045029 | −0.91423505 |
| SLC2A4RG | −0.9095737 | −0.59876869 |
| LIME1 | −0.90957369 | −0.59876869 |
| NAT2 | −0.48889312 | −0.90882634 |
| MME | −0.78982822 | −0.90698252 |
| APOC3 | −0.89246412 | −0.45008406 |
| APOA1 | −0.89246411 | −0.45008404 |
| GCGR | −0.89023552 | −0.62501281 |
| ACADS | −0.78259387 | −0.88482951 |
| IGFALS | −0.88450169 | −0.6259388 |
| CPNE6 | −0.87891263 | −0.80760341 |
| CYP2E1 | −0.73994513 | −0.87852408 |
| RP4-601P9.2 | −0.77082548 | −0.87729331 |
| THOP1 | −0.86939272 | −0.74055984 |
| GCDH | −0.86451674 | −0.61958447 |
| FAM151A | −0.83402025 | −0.85612392 |
| LINC00844 | −0.64764145 | −0.84587488 |
| AOX1 | −0.43160766 | −0.84293406 |
| TBX3 | −0.63799608 | −0.84108221 |
| TRIM55 | −0.41269109 | −0.84011324 |
| SLC22A25 | −0.61387539 | −0.83916424 |
| RBP4 | −0.83168116 | −0.72002451 |
| RP11-6B4.1 | −0.48612421 | −0.82930402 |
| PRODH2 | −0.82671595 | −0.78277324 |
| PCOLCE2 | −0.67769587 | −0.8229104 |
| CXXC4 | −0.49948404 | −0.82209373 |
| ORM2 | −0.48125488 | −0.82026193 |
| TRPC5 | −0.43558564 | −0.81958049 |
| GPR88 | −0.81941466 | −0.61700657 |
| CNPY3 | −0.81711475 | −0.71387346 |
| PPP1R32 | −0.81701208 | −0.54806826 |
| FITM1 | −0.6639242 | −0.81316963 |
| C1orf226 | −0.72632656 | −0.81225523 |
| PDLIM1P4 | −0.44372511 | −0.81068592 |
| RGN | −0.8093521 | −0.64054053 |
| C3P1 | −0.67831697 | −0.80805497 |
| ETNK2 | −0.80482527 | −0.67150957 |
| CES1P1 | −0.79369268 | −0.80408941 |
| NAGS | −0.80097203 | −0.71944772 |
| ZGPAT | −0.79965053 | −0.51234221 |
| FADS6 | −0.73288221 | −0.79342869 |
| HAO2-IT1 | −0.59955733 | −0.79336794 |
| KBTBD11 | −0.40521894 | −0.79100922 |
| MAGI2-AS3 | −0.35584842 | −0.78285799 |
| SLC7A9 | −0.78097086 | −0.76962553 |
| GFRA1 | −0.55440634 | −0.78061658 |
| CTD-2529021.2 | −0.53112617 | −0.78012415 |
| SNTG1 | −0.55097541 | −0.77969493 |
| RP11-113122.1 | −0.7130455 | −0.77938329 |
| MEX3A | −0.522985 | −0.77832605 |
| TYK2 | −0.65803804 | −0.7777852 |
| MOGAT2 | −0.71494902 | −0.77733915 |
| Y_RNA | −0.57640779 | −0.77705699 |
| SMO | −0.69834739 | −0.77436359 |
| RP11-475O6.1 | −0.49405245 | −0.77418792 |
| RP11-115J16.1 | −0.55973102 | −0.77241533 |
| RP11-417L19.2 | −0.77042498 | −0.62416275 |
| ALDH1L1-AS2 | −0.63925942 | −0.77040614 |
| AR | −0.59966299 | −0.76993595 |
| WFIKKN1 | −0.76945835 | −0.60842709 |
| FAM35BP | −0.72495655 | −0.76611146 |
| RP11-38L15.8 | −0.72495294 | −0.76610895 |
| CYP2D6 | −0.76392889 | −0.54764712 |
| ADH4 | −0.62470979 | −0.762872 |
| SERPINC1 | −0.64231174 | −0.75788513 |
| GPER1 | −0.73874659 | −0.7562845 |
| FAM99B | −0.75424502 | −0.63999416 |
| TSC2 | −0.75369796 | −0.6199147 |
| RP11-122K13.7 | −0.75356381 | −0.54125037 |
| PRAP1 | −0.75356381 | −0.54125041 |
| FUOM | −0.75356381 | −0.54125046 |
| CHAD | −0.74741586 | −0.49513365 |
| APOC2 | −0.7450738 | −0.54355247 |
| SLCO1B3 | −0.58591714 | −0.7442032 |
| ALDH1L1 | −0.59999491 | −0.74407132 |
| FMO3 | −0.49424232 | −0.74299001 |
| TM6SF2 | −0.73970533 | −0.62457193 |
| CAMSAP3 | −0.7391082 | −0.64069753 |
| RP11-403I13.4 | −0.53561344 | −0.73909758 |
| RP11-7F17.3 | −0.73628466 | −0.7269955 |
| RP11-830F9.5 | −0.7359723 | −0.57756223 |
| RP11-119D9.1 | −0.66797538 | −0.73584138 |
| ECHDC3 | −0.73574499 | −0.64695488 |
| GJB1 | −0.73271966 | −0.71216975 |
| GSTA7P | −0.69554599 | −0.72836277 |
| C11orf95 | −0.66759182 | −0.72831986 |
| MTND4P20 | −0.53622291 | −0.72807145 |
| LINC01018 | −0.72663526 | −0.4670925 |
| CTD-2227E11.1 | −0.72581509 | −0.72646364 |
| HAGH | −0.7241312 | −0.56129832 |
| RP11-372E1.4 | −0.48838126 | −0.72382996 |
| RP11-706C16.7 | −0.72151361 | −0.67126525 |
| JAKMIP2 | −0.59190911 | −0.71988615 |
| AP006216.5 | −0.71728566 | −0.60883583 |
| HPX | −0.53134133 | −0.71341157 |
| ALB | −0.61593953 | −0.71214067 |
| RP5-881L22.6 | −0.71205592 | −0.62633386 |
| PLEK2 | −0.58279018 | −0.71204221 |
| HGFAC | −0.51325387 | −0.71165281 |
| ASB13 | −0.70086639 | −0.71154238 |
| GSTA1 | −0.71032899 | −0.64856362 |
| DGAT2 | −0.70679534 | −0.70974109 |
| NAT1 | −0.70961995 | −0.68535333 |
| RP1-152L7.5 | −0.60024854 | −0.7088288 |
| PLGLA | −0.52861767 | −0.70862097 |
| CBLN4 | −0.68430856 | −0.70843115 |
| AASS | −0.56644207 | −0.70571741 |
| CYP4A11 | −0.705453 | −0.56334321 |
| MIR5589 | −0.53586591 | −0.70311148 |
| GALK1 | −0.6524941 | −0.70208265 |
| RP11-260M19.2 | −0.7011436 | −0.6485646 |
| AC005077.7 | −0.46807058 | −0.7006907 |
| EFNA2 | −0.49300466 | −0.69998303 |
| LYNX1 | −0.69971324 | −0.67462418 |
| SPSB3 | −0.69903937 | −0.46642679 |
| KCNMA1 | −0.55430532 | −0.69530983 |
| C10orf11 | −0.29734433 | −0.69480888 |
| RP11-659E9.2 | −0.66184955 | −0.69454529 |
| CECR2 | −0.54792984 | −0.69282446 |
| ADRA1A | −0.4610272 | −0.68963291 |
| APOA5 | −0.68897857 | −0.46835311 |
| IGF2 | −0.68669975 | −0.61636773 |
| INS-IGF2 | −0.68669973 | −0.61636774 |
| AADAT | −0.50497899 | −0.68563358 |
| CTD-2587M2.1 | −0.68322033 | −0.65519024 |
| SLC10A1 | −0.68208719 | −0.60260581 |
| TMPRSS6 | −0.67892758 | −0.46227989 |
| CTC-575D19.1 | −0.50301008 | −0.67783699 |
| SCP2 | −0.54729091 | −0.67775912 |
| NR1I2 | −0.67700905 | −0.62030923 |
| ABAT | −0.33331643 | −0.67659891 |
| GS1-124K5.11 | −0.6751906 | −0.64925827 |
| ECHDC2 | −0.54473282 | −0.67350042 |
| PON3 | −0.58047417 | −0.6721999 |
| HAO2 | −0.58929896 | −0.6716787 |
| SLC22A7 | −0.66730949 | −0.51135093 |
| SLC9A3R2 | −0.66600375 | −0.49863254 |
| GRHPR | −0.6657719 | −0.47558544 |
| SLC25A47 | −0.66548534 | −0.44343802 |
| CFHR5 | −0.39484139 | −0.66518336 |
| PLG | −0.52855394 | −0.66518177 |
| AZGP1 | −0.62395669 | −0.66494299 |
| PCSK9 | −0.60364697 | −0.66493919 |
| NTHL1 | −0.66491983 | −0.49255849 |
| APOC1 | −0.66305233 | −0.48365484 |
| APOE | −0.66305233 | −0.48365476 |
| AZGP1P1 | −0.66194605 | −0.54918409 |
| F2 | −0.46835274 | −0.66110168 |
| DFFB | −0.61016315 | −0.66016317 |
| MAT1A | −0.47979927 | −0.65852281 |
| EVPLL | −0.54608141 | −0.6559937 |
| MFSD3 | −0.65447539 | −0.44261957 |
| GPT | −0.65447539 | −0.44261959 |
| SMLR1 | −0.52661369 | −0.6541607 |
| PPP1R1C | −0.48262948 | −0.65398528 |
| CTD-2517M22.14 | −0.65335043 | −0.4442219 |
| SULT1A1 | −0.65307806 | −0.54881671 |
| ST3GAL6 | −0.29637856 | −0.6524712 |
| SLC25A34 | −0.65212305 | −0.54308214 |
| MGMT | −0.46699718 | −0.65200609 |
| ZNF511 | −0.65139548 | −0.4622824 |
| TMEM105 | −0.55302581 | −0.65100671 |
| HSD17B10 | −0.65077375 | −0.54606299 |
| RP3-339A18.6 | −0.65077365 | −0.54606314 |
| SERPINF2 | −0.61094679 | −0.65017548 |
| FBLN7 | −0.65001246 | −0.38974033 |
| SLC39A5 | −0.64976858 | −0.55007742 |
| SYAP1 | −0.4535541 | −0.64927048 |
| RP13-650J16.1 | −0.6478358 | −0.3982245 |
| DCXR | −0.64783579 | −0.39822448 |
| TPCN2 | −0.64773178 | −0.59461018 |
| PON1 | −0.59295438 | −0.64760568 |
| FAHD1 | −0.64633858 | −0.48634266 |
| FOXP2 | −0.33860001 | −0.64592897 |
| AZGP1P2 | −0.64485418 | −0.53287999 |
| TRABD2B | −0.47722253 | −0.64321508 |
| PROX1-AS1 | −0.33832987 | −0.64270413 |
| C2orf72 | −0.5594156 | −0.6418142 |
| GNA11 | −0.57685342 | −0.64092872 |
| HAAO | −0.6387934 | −0.45134397 |
| APOH | −0.43648694 | −0.63846139 |
| THAP3 | −0.50147004 | −0.63719817 |
| SLC38A3 | −0.63662377 | −0.36577035 |
| AP006285.7 | −0.63650096 | −0.51105337 |
| RP11-223I10.1 | −0.57321173 | −0.6353221 |
| MSRB1 | −0.4839175 | −0.63462722 |
| SEMA4G | −0.63420912 | −0.46527582 |
| UGT2B7 | −0.34735527 | −0.63379328 |
| COL18A1 | −0.633764 | −0.48613968 |
| TTR | −0.60446147 | −0.63364811 |
| GC | −0.29478957 | −0.6335266 |
| SLC6A12 | −0.6333656 | −0.45859746 |
| MT1X | −0.44253318 | −0.63240484 |
| HPGD | −0.4128829 | −0.63133765 |
| SKP2 | −0.35220983 | −0.62910207 |
| SULT1E1 | −0.40411073 | −0.62873206 |
| GOT2 | −0.62852963 | −0.53663294 |
| PROX1 | −0.35402263 | −0.62818618 |
| LCAT | −0.62813838 | −0.51929613 |
| TP53I13 | −0.62703429 | −0.37204121 |
| CES3 | −0.58736479 | −0.626415 |
| KLF15 | −0.62635639 | −0.45928035 |
| BHMT | −0.62591419 | −0.55159714 |
| HSD17B6 | −0.52637047 | −0.62578294 |
| CYP2C8 | −0.45076188 | −0.62565461 |
| MASP2 | −0.62540411 | −0.45429021 |
| FTCD | −0.62522253 | −0.38500405 |
| ABCC6P1 | −0.62509976 | −0.38452141 |
| TDO2 | −0.35564566 | −0.62458298 |
| SEPP1 | −0.39355054 | −0.62306905 |
| PSAT1 | −0.54753136 | −0.62300561 |
| AC016768.1 | −0.38420885 | −0.62272919 |
| ESRP2 | −0.6218496 | −0.48248855 |
| REXO1 | −0.56277739 | −0.62150134 |
| BCKDHB | −0.62123979 | −0.4713873 |
| WNK3 | −0.62106763 | −0.34570911 |
| GYS2 | −0.41303876 | −0.62103511 |
| SULT1A2 | −0.62028062 | −0.48991151 |
| TOMM40 | −0.61989376 | −0.43165585 |
| GRB14 | −0.4458305 | −0.61988769 |
| ALDH7A1 | −0.46238695 | −0.61876846 |
| SEC14L4 | −0.61755403 | −0.41089817 |
| MLXIPL | −0.6175478 | −0.54816044 |
| RAC3 | −0.61724767 | −0.35175161 |
| NUGGC | −0.44890558 | −0.61652604 |
| F7 | −0.61648164 | −0.50799424 |
| RTP3 | −0.61626652 | −0.54756019 |
| CES1 | −0.61619799 | −0.41731858 |
| RFNG | −0.61435967 | −0.50350499 |
| GPS1 | −0.61435961 | −0.50350491 |
| PPP1R16A | −0.61345434 | −0.41105207 |
| TTBK1 | −0.60395821 | −0.61324208 |
| KLHDC10 | −0.61303155 | −0.60010566 |
| MYO15A | −0.61245206 | −0.50874414 |
| RP5-834N19.1 | −0.51365436 | −0.61200935 |
| GAMT | −0.61187939 | −0.36664478 |
| IYD | −0.50358548 | −0.61132638 |
| MPST | −0.6108748 | −0.54581605 |
| RP11-418J17.3 | −0.57473442 | −0.61052084 |
| CPN2 | −0.48459523 | −0.6103631 |
| PSMB7 | −0.40820418 | −0.61021229 |
| PPARA | −0.6100155 | −0.52676745 |
| SPP2 | −0.56862656 | −0.60999811 |
| HFE2 | −0.46933974 | −0.60889644 |
| DMRTA1 | −0.51210504 | −0.60876512 |
| SORD | −0.58079901 | −0.60821283 |
| FAM99A | −0.608083 | −0.52127727 |
| XAB2 | −0.60789085 | −0.53251592 |
| GPRC5C | −0.60748968 | −0.58039232 |
| PKD2 | −0.44771588 | −0.6074226 |
| ACOT6 | −0.60734884 | −0.41927042 |
| COX10-AS1 | −0.53103449 | −0.60731393 |
| ST18 | −0.40357472 | −0.6066086 |
| NRBP2 | −0.6050793 | −0.55679106 |
| ABCC11 | −0.40158633 | −0.60456873 |
| PGRMC1 | −0.47252679 | −0.60425638 |
| PLIN4 | −0.60248646 | −0.60349035 |
| FES | −0.42565685 | −0.60271729 |
| SLC45A3 | −0.38899675 | −0.60209738 |
| TMEM176A | −0.46405227 | −0.60127865 |
| MBL2 | −0.53147691 | −0.60088917 |
| SLC16A2 | −0.33244141 | −0.60064312 |
| ADH1B | −0.43132286 | −0.60005989 |
| NOS1AP | −0.40807855 | −0.59979899 |
| SLC44A1 | −0.36490248 | −0.59952296 |
| TDGF1 | −0.41829492 | −0.59947195 |
| ACSM5 | −0.4846508 | −0.59940731 |
| DHTKD1 | −0.59913906 | −0.4510699 |
| ANPEP | −0.39222731 | −0.59797437 |
| AP000695.6 | −0.59793891 | −0.58609996 |
| TMEM110-MUSTN1 | −0.48091903 | −0.59739318 |
| MIR3646 | −0.5959499 | −0.52518315 |
| NUDC | −0.59416921 | −0.54419999 |
| TMEM176B | −0.45821951 | −0.59377894 |
| DGAT1 | −0.59367306 | −0.38374643 |
| CNTLN | −0.38339951 | −0.59294026 |
| ASGR2 | −0.57713067 | −0.59260218 |
| NLRP14 | −0.47829452 | −0.59257624 |
| RAVER1 | −0.58965445 | −0.56794636 |
| PLIN5 | −0.43688193 | −0.58939783 |
| RAPSN | −0.44810686 | −0.58914135 |
| KHK | −0.58850678 | −0.47178375 |
| NBPF13P | −0.52308524 | −0.58820299 |
| CEACAM22P | −0.58759132 | −0.5714708 |
| SLC43A1 | −0.58716714 | −0.48673092 |
| HNF1A | −0.58703441 | −0.43191851 |
| CMYA5 | −0.46678244 | −0.586006 |
| PQLC1 | −0.51128552 | −0.58518879 |
| LRRC3 | −0.58506819 | −0.33908631 |
| LRRC3-AS1 | −0.58506818 | −0.33908633 |
| CHID1 | −0.52113529 | −0.58487534 |
| KLKB1 | −0.49699917 | −0.58477479 |
| CTB-50L17.14 | −0.42648991 | −0.58312503 |
| HSD17B14 | −0.58311868 | −0.52414515 |
| MGST1 | −0.44586517 | −0.58266921 |
| ARID3C | −0.58266304 | −0.51941336 |
| CAT | −0.58201229 | −0.48705715 |
| RP11-252E2.2 | −0.58143053 | −0.56129588 |
| SLC22A10 | −0.53408945 | −0.58121634 |
| ACOX2 | −0.5805449 | −0.51146067 |
| RP11-696N14.1 | −0.44797812 | −0.58037082 |
| GLYAT | −0.41431923 | −0.57973952 |
| NR2F6 | −0.5793919 | −0.40638654 |
| RNF128 | −0.36331509 | −0.57910884 |
| C1orf115 | −0.49538995 | −0.57895323 |
| SNAPC4 | −0.57830369 | −0.36224395 |
| SNED1 | −0.57740037 | −0.40034854 |
| PNPLA3 | −0.5771948 | −0.50713825 |
| SCG5 | −0.50820222 | −0.57715739 |
| NIT2 | −0.52382134 | −0.57681417 |
| TST | −0.57610113 | −0.47748132 |
| GGACT | −0.29750282 | −0.57565388 |
| GSTZ1 | −0.57416671 | −0.49594461 |
| MRPL43 | −0.57393552 | −0.38562571 |
| SLC27A5 | −0.57384955 | −0.36274605 |
| BCAT2 | −0.57362287 | −0.4821496 |
| POLE | −0.5734815 | −0.36457824 |
| SERPINA4 | −0.45149394 | −0.57326362 |
| ABCG8 | −0.57311013 | −0.43992538 |
| TEF | −0.57245391 | −0.41824675 |
| LBX2 | −0.54741405 | −0.571731 |
| LBX2-AS1 | −0.54741406 | −0.57173079 |
| RP11-523H20.3 | −0.54741405 | −0.57173062 |
| BPHL | −0.37664424 | −0.57086038 |
| PACSIN3 | −0.51016493 | −0.569775 |
| ASPG | −0.5695377 | −0.44994196 |
| SERPINA5 | −0.37963361 | −0.56949765 |
| CA14 | −0.56920372 | −0.47864338 |
| GGH | −0.30389191 | −0.56847041 |
| AKRID1 | −0.46639569 | −0.56766263 |
| HNF1A-AS1 | −0.56746377 | −0.42809642 |
| PKD1 | −0.56703768 | −0.35638666 |
| C8orf82 | −0.56627989 | −0.3824422 |
| TTC36 | −0.55970467 | −0.56589728 |
| CLDN14 | −0.51014848 | −0.5653727 |
| PROC | −0.46263394 | −0.56342543 |
| HNF4A | −0.56170072 | −0.52228426 |
| KCTD21-AS1 | −0.37965635 | −0.56160309 |
| NDUFA6-AS1 | −0.56092034 | −0.38097628 |
| IVD | −0.56033217 | −0.43510994 |
| SLC25A10 | −0.55948616 | −0.36553108 |
| DNAJC12 | −0.40517798 | −0.55921823 |
| ITIH4 | −0.48430675 | −0.55896096 |
| RP5-966M1.6 | −0.48430678 | −0.5589609 |
| CBS | −0.55818014 | −0.49263627 |
| VIL1 | −0.55804691 | −0.50261453 |
| FTCDNL1 | −0.55759976 | −0.42851501 |
| TMEM25 | −0.53650468 | −0.55661602 |
| ALAD | −0.55652649 | −0.4414033 |
| POMT2 | −0.55575725 | −0.47829184 |
| SLC19A1 | −0.55514766 | −0.39980198 |
| AK4 | −0.37776613 | −0.55510821 |
| MOGAT1 | −0.46931272 | −0.55422142 |
| PCYT2 | −0.55361709 | −0.37385382 |
| RP4-758J18.2 | −0.55342691 | −0.33293506 |
| CCNL2 | −0.5534269 | −0.33293503 |
| QDPR | −0.41312051 | −0.55337415 |
| GNAO1 | −0.40709817 | −0.55335288 |
| SAT2 | −0.55328098 | −0.32899268 |
| NUDT16 | −0.49638324 | −0.55146716 |
| ABHD15 | −0.55087095 | −0.2706481 |
| RP3-402G11.26 | −0.55049535 | −0.42967261 |
| SELO | −0.55049531 | −0.42967262 |
| TMEM110 | −0.4523131 | −0.54941938 |
| AMBP | −0.5332563 | −0.54935059 |
| PHGDH | −0.39295342 | −0.54867972 |
| ALKBH2 | −0.53423372 | −0.54812573 |
| XYLB | −0.54782416 | −0.46845163 |
| TTC31 | −0.54772171 | −0.40042391 |
| C1R | −0.36476152 | −0.54761051 |
| STEAP3 | −0.37738984 | −0.54733639 |
| PMM1 | −0.54627888 | −0.28253201 |
| C16orf13 | −0.54570672 | −0.32688719 |
| ITIH1 | −0.44793049 | −0.54564737 |
| SHMT2 | −0.54552397 | −0.52298954 |
| SH3PXD2A | −0.44187639 | −0.54539559 |
| ASL | −0.54456235 | −0.52231389 |
| NDUFS7 | −0.54417121 | −0.29338413 |
| NAPEPLD | −0.42271619 | −0.54372659 |
| TCEA3 | −0.45619391 | −0.54341951 |
| SUGP1 | −0.54098686 | −0.44638745 |
| VTN | −0.54075469 | −0.443821 |
| SHANK3 | −0.54064551 | −0.39602555 |
| ECHS1 | −0.54056713 | −0.41448155 |
| MFAP3L | −0.30652926 | −0.53937362 |
| MRPL23 | −0.53932617 | −0.50045197 |
| MIR126 | −0.53192407 | −0.53928628 |
| AGXT2 | −0.32841234 | −0.53910873 |
| SLC25A42 | −0.53807907 | −0.36450899 |
| MARC1 | −0.38312721 | −0.53739454 |
| ELP3 | −0.36883554 | −0.53613072 |
| DAB1 | −0.32375416 | −0.53608108 |
| TMEM37 | −0.3196043 | −0.53540935 |
| GULOP | −0.53516416 | −0.40178378 |
| ASPSCR1 | −0.53480527 | −0.32653753 |
| GNE | −0.45806797 | −0.53442947 |
| SLC25A18 | −0.50350814 | −0.53424868 |
| SLC46A1 | −0.53407199 | −0.42455711 |
| SFXN5 | −0.496014 | −0.53339116 |
| C3 | −0.32496828 | −0.53334588 |
| ICAM3 | −0.5330691 | −0.47745957 |
| TTPAL | −0.53285799 | −0.4357361 |
| CTNS | −0.53240924 | −0.37648291 |
| PRKAG2-AS1 | −0.5310375 | −0.31160358 |
| ABCG5 | −0.5300833 | −0.41424045 |
| PROZ | −0.52983523 | −0.46086358 |
| SUOX | −0.47224945 | −0.52936307 |
| FAM53A | −0.52900374 | −0.37347314 |
| SPTBN2 | −0.41482506 | −0.52846757 |
| CASC10 | −0.46069097 | −0.52801376 |
| LCN12 | −0.52755957 | −0.2416872 |
| C8G | −0.52755957 | −0.2416872 |
| SIGMAR1 | −0.52753124 | −0.29635608 |
| MLYCD | −0.52746589 | −0.40467836 |
| CPPED1 | −0.45585123 | −0.52692497 |
| APOF | −0.39252114 | −0.52685065 |
| ABCC2 | −0.33069775 | −0.526547 |
| HPN | −0.52571552 | −0.45549562 |
| PKLR | −0.52544006 | −0.47379075 |
| RAD54L2 | −0.52459046 | −0.48270611 |
| RP5-875H18.4 | −0.52423609 | −0.39216451 |
| CMBL | −0.50367322 | −0.52370941 |
| RP11-390F4.2 | −0.33177001 | −0.52181134 |
| SLC30A10 | −0.38143031 | −0.52019655 |
| ACADSB | −0.35733082 | −0.51715283 |
| ITCH | −0.36434796 | −0.51632268 |
| RP11-1151B14.3 | −0.39617577 | −0.51550649 |
| RP11-661A12.7 | −0.51475978 | −0.36138695 |
| SLC13A5 | −0.44023114 | −0.51468042 |
| TMEM220 | −0.44426325 | −0.51422255 |
| PRKAG2 | −0.51333418 | −0.37661849 |
| TADA1 | −0.51288083 | −0.42028579 |
| ASGR1 | −0.5127561 | −0.48431379 |
| DOLPP1 | −0.51267896 | −0.41169338 |
| FXYD1 | −0.51259674 | −0.34985605 |
| PCK2 | −0.51153711 | −0.38174507 |
| DHRS4 | −0.51151351 | −0.35312145 |
| PGLYRP2 | −0.51129907 | −0.43756266 |
| LPAL2 | −0.47613199 | −0.51093138 |
| AC142528.1 | −0.50993273 | −0.47855001 |
| AGPAT2 | −0.50987929 | −0.50145303 |
| PEBP1 | −0.50957342 | −0.36946543 |
| HIBADH | −0.40473344 | −0.50932067 |
| CYP4F3 | −0.50927874 | −0.48591038 |
| RGS12 | −0.41474877 | −0.50878816 |
| TNFAIP8L1 | −0.50681237 | −0.31462028 |
| RP11-407B7.1 | −0.36462574 | −0.50655877 |
| DCTD | −0.38206799 | −0.5062648 |
| MRPS22 | −0.42399493 | −0.50613564 |
| DBT | −0.35164339 | −0.50496922 |
| PAOX | −0.50466519 | −0.27578744 |
| PAIP2B | −0.50458813 | −0.43717477 |
| MPC1 | −0.32317742 | −0.50313087 |
| NDRG2 | −0.38817639 | −0.5029702 |
| PEMT | −0.47849196 | −0.50282158 |
| ALDH8A1 | −0.30685945 | −0.50167267 |
| TSSC1 | −0.41604199 | −0.50166131 |
| AP001065.15 | −0.50121439 | −0.43630473 |
| TSPAN9 | −0.39052741 | −0.5010383 |
| FGFR4 | −0.50079069 | −0.40955859 |
| ZNF444 | −0.49924193 | −0.47143732 |
| LINC00094 | −0.49907735 | −0.34613862 |
| FAM73B | −0.49864561 | −0.41579389 |
| TTPA | −0.29893692 | −0.49838552 |
| CTD-2545H1.2 | −0.49794342 | −0.4922324 |
| TFR2 | −0.49737757 | −0.40044049 |
| FLCN | −0.49727171 | −0.39699181 |
| ACAA1 | −0.49691562 | −0.40187225 |
| FURIN | −0.38736648 | −0.49665955 |
| SALL1 | −0.32368911 | −0.49658827 |
| ZNF696 | −0.49657629 | −0.3440484 |
| RNF126 | −0.49601691 | −0.33194009 |
| AC004156.3 | −0.49601688 | −0.33194018 |
| POLR3H | −0.49517761 | −0.31785751 |
| GHR | −0.32229072 | −0.49490278 |
| ACP2 | −0.49474027 | −0.46615352 |
| ZNF497 | −0.49442209 | −0.39183096 |
| ZBTB48 | −0.49401346 | −0.2997022 |
| FAM20C | −0.49390927 | −0.46207081 |
| NAT8 | −0.4935748 | −0.42309711 |
| HPD | −0.49355526 | −0.42777177 |
| ADI1 | −0.49351634 | −0.42814468 |
| CTA-292E10.6 | −0.2790415 | −0.49296135 |
| SNHG8 | −0.30963332 | −0.49265941 |
| BNIP3 | −0.49251914 | −0.36536377 |
| ZNF517 | −0.4917722 | −0.46460047 |
| EHHADH | −0.41997315 | −0.49164704 |
| RP11-449P15.2 | −0.49137643 | −0.43785462 |
| GET4 | −0.4913764 | −0.43785455 |
| TBC1D2 | −0.44369124 | −0.49087881 |
| SFXN1 | −0.45312962 | −0.49081025 |
| FBXW11P1 | −0.42475793 | −0.49075521 |
| TTC6 | −0.2733433 | −0.49007905 |
| GALT | −0.48939574 | −0.32661027 |
| MRPL20 | −0.48889795 | −0.29298016 |
| RP11-513G11.3 | −0.32566644 | −0.48866387 |
| ZBTB45 | −0.48783977 | −0.30485582 |
| EDC3 | −0.44603223 | −0.48753583 |
| AC009166.5 | −0.32444132 | −0.48751622 |
| PCBD1 | −0.48719245 | −0.39835197 |
| RP11-44M6.3 | −0.48716407 | −0.36623863 |
| LRRC37A5P | −0.4311686 | −0.48715786 |
| OSGIN1 | −0.48661609 | −0.33313549 |
| DEPDC7 | −0.3491709 | −0.48657689 |
| TMEM82 | −0.48639705 | −0.40000723 |
| SLC26A1 | −0.48606527 | −0.39855654 |
| HSBP1L1 | −0.35304183 | −0.48601142 |
| THRB | −0.29114288 | −0.48535447 |
| ENTPD5 | −0.35027874 | −0.48523689 |
| PKD1L2 | −0.46030085 | −0.48456053 |
| BDH1 | −0.48419263 | −0.33955255 |
| SLC25A20 | −0.48393904 | −0.34051564 |
| RDH16 | −0.48359387 | −0.4489049 |
| MYD88 | −0.48334123 | −0.39550524 |
| MPLKIP | −0.48304731 | −0.40127274 |
| FBP1 | −0.48272264 | −0.35558511 |
| SOWAHB | −0.46464072 | −0.48157392 |
| FUBP3 | −0.48155248 | −0.40610461 |
| CYP27A1 | −0.41327242 | −0.48118186 |
| IL11RA | −0.4798205 | −0.3337762 |
| SLC27A2 | −0.44706769 | −0.47901059 |
| ZNF385B | −0.33023531 | −0.47848627 |
| DEAF1 | −0.47796184 | −0.36825415 |
| POLR2F | −0.47774132 | −0.33296713 |
| MMACHC | −0.47712778 | −0.41091257 |
| RP11-344P13.4 | −0.39852927 | −0.47679292 |
| IDUA | −0.47671009 | −0.39321773 |
| HDHD3 | −0.44181024 | −0.47659129 |
| FABP1 | −0.30753758 | −0.47627108 |
| SCCPDH | −0.45745009 | −0.47546018 |
| EPHX2 | −0.47481538 | −0.43637706 |
| TTC38 | −0.474034 | −0.28784079 |
| PCDH1 | −0.27460496 | −0.47387188 |
| C19orf66 | −0.47381786 | −0.43815769 |
| CFB | −0.35693341 | −0.47336005 |
| MTHFD1 | −0.46427643 | −0.47210587 |
| CYP8B1 | −0.32041968 | −0.47112259 |
| TRIM24 | −0.40642724 | −0.46963243 |
| FKBP8 | −0.46922264 | −0.43147395 |
| PEX6 | −0.46908041 | −0.33063488 |
| BTD | −0.38310231 | −0.46782556 |
| SLC6A1 | −0.46749302 | −0.33398139 |
| RANBP1 | −0.46739615 | −0.34553256 |
| RP11-38G5.4 | −0.44114597 | −0.4670968 |
| TOLLIP | −0.4670631 | −0.37155505 |
| LGI4 | −0.46690066 | −0.30048316 |
| RP11-209K10.2 | −0.35233705 | −0.46683615 |
| OPLAH | −0.44651345 | −0.46490928 |
| SGK2 | −0.39416895 | −0.46457315 |
| EI24 | −0.46454905 | −0.3812715 |
| CYP4F11 | −0.4083028 | −0.46418502 |
| WDR18 | −0.40553702 | −0.46347861 |
| TOLLIP-AS1 | −0.463264 | −0.37064645 |
| USP30 | −0.45958095 | −0.46283748 |
| ABCG2 | −0.45366629 | −0.46181431 |
| ACACB | −0.35797062 | −0.46176925 |
| AHCY | −0.4064863 | −0.46103644 |
| ABCC6 | −0.43846415 | −0.45989323 |
| GLS2 | −0.45963971 | −0.27880136 |
| AC019181.2 | −0.39783467 | −0.45961561 |
| ACVR1C | −0.38045717 | −0.45891588 |
| F10 | −0.45882069 | −0.36950754 |
| ZKSCAN1 | −0.39867916 | −0.45840621 |
| ADCY1 | −0.45809579 | −0.37976781 |
| CHMP6 | −0.42696208 | −0.4575594 |
| RPL7AP6 | −0.35534184 | −0.4563226 |
| SEC14L2 | −0.43225002 | −0.45577579 |
| C7orf50 | −0.45567089 | −0.36783366 |
| FAH | −0.36603446 | −0.45532692 |
| CLUH | −0.45525342 | −0.26882068 |
| PPP6R2 | −0.45511075 | −0.25961048 |
| C1S | −0.27588469 | −0.45447322 |
| FANCC | −0.28011776 | −0.45402927 |
| SLC25A13 | −0.45387704 | −0.43150595 |
| RAPH1 | −0.31973935 | −0.45347927 |
| RNF215 | −0.45336741 | −0.45261625 |
| PAH | −0.36803375 | −0.45335418 |
| GCSH | −0.45281308 | −0.34995596 |
| LHPP | −0.45207122 | −0.33877499 |
| TFDP2 | −0.45187246 | −0.39525617 |
| KDM8 | −0.39823294 | −0.45164846 |
| ACSF2 | −0.45161505 | −0.28630564 |
| RAB26 | −0.45154017 | −0.29370101 |
| SMOC1 | −0.40655165 | −0.45031917 |
| AL161668.5 | −0.4499014 | −0.32396975 |
| TPPP2 | −0.44990139 | −0.3239697 |
| IPO5 | −0.26741723 | −0.44948608 |
| ZC3H7B | −0.44913537 | −0.3385584 |
| RP4-584D14.7 | −0.3750442 | −0.44912805 |
| RARRES2 | −0.3750443 | −0.44912805 |
| POLM | −0.44893481 | −0.29853335 |
| RP11-713M15.2 | −0.38277897 | −0.44866948 |
| GPR146 | −0.44865796 | −0.38895443 |
| MOCS1 | −0.41773577 | −0.44792101 |
| SERPIND1 | −0.36698026 | −0.44786657 |
| TACO1 | −0.44688164 | −0.37216997 |
| EPHX1 | −0.4466512 | −0.2775992 |
| SDC2 | −0.3101171 | −0.44600249 |
| RP11-45M22.4 | −0.44589988 | −0.30248336 |
| TOP1MT | −0.39726686 | −0.44579036 |
| FAAH | −0.44572098 | −0.40437451 |
| SLC22A1 | −0.35592978 | −0.44505431 |
| CTD-2619J13.8 | −0.44504645 | −0.33025985 |
| PUF60 | −0.44404391 | −0.29095008 |
| GLYCTK | −0.44377755 | −0.35026885 |
| ARFGAP2 | −0.44372545 | −0.35288168 |
| PXMP2 | −0.4433802 | −0.24999398 |
| RAB40C | −0.44330146 | −0.31355658 |
| PPL | −0.4315543 | −0.44327275 |
| ESD | −0.39666646 | −0.44284912 |
| IQGAP2 | −0.32689737 | −0.44264569 |
| DECR2 | −0.3407892 | −0.44198449 |
| ARMC6 | −0.44171579 | −0.38459195 |
| POLE2 | −0.43139487 | −0.44093522 |
| CDO1 | −0.44002295 | −0.33446279 |
| ITIH3 | −0.34223843 | −0.43953042 |
| SP5 | −0.43825549 | −0.42995769 |
| MAL2 | −0.32726125 | −0.43704003 |
| SMARCA1 | −0.29755465 | −0.43694811 |
| AP1M1 | −0.42285109 | −0.43687256 |
| SH2D3A | −0.39439644 | −0.43681467 |
| CLSTN3 | −0.35954896 | −0.43629611 |
| RP11-736K20.6 | −0.35310608 | −0.43597212 |
| MAOB | −0.35851329 | −0.43468652 |
| ACSM3 | −0.40360969 | −0.43359854 |
| PITPNM2 | −0.43183173 | −0.40867964 |
| ALDH6A1 | −0.43183011 | −0.35096677 |
| EPB41L4B | −0.43182827 | −0.32919962 |
| SCLY | −0.40008884 | −0.43091631 |
| OAF | −0.33562809 | −0.43048654 |
| ALDH4A1 | −0.28716605 | −0.43042383 |
| ARRDC1 | −0.43038866 | −0.31724026 |
| KCTD21 | −0.33341511 | −0.43004666 |
| MTSS1 | −0.33017069 | −0.42971923 |
| RP11-273B20.1 | −0.42903237 | −0.38342987 |
| RBP5 | −0.42903235 | −0.38342977 |
| AGL | −0.30940841 | −0.42726395 |
| AKT2 | −0.42725154 | −0.30639023 |
| DHRS1 | −0.31698678 | −0.42714246 |
| SS18L1 | −0.29773655 | −0.42702242 |
| LDHD | −0.42515427 | −0.42699012 |
| CNGA1 | −0.4261871 | −0.37338182 |
| CYB5A | −0.30128304 | −0.42584528 |
| GPD1 | −0.42512576 | −0.33581025 |
| CRLS1 | −0.38554414 | −0.42500853 |
| EEF1D | −0.42455967 | −0.27087658 |
| RNF43 | −0.33079245 | −0.42327577 |
| SND1 | −0.41353442 | −0.42320194 |
| STARD10 | −0.42288169 | −0.30011581 |
| UNC119B | −0.2635271 | −0.42255029 |
| CYP4F2 | −0.36909994 | −0.42188912 |
| RAPGEFL1 | −0.42174714 | −0.38579213 |
| CCS | −0.42145656 | −0.35268149 |
| C1RL | −0.28282706 | −0.42072201 |
| RABEPK | −0.25830292 | −0.42060896 |
| SLC25A1 | −0.42051755 | −0.29538267 |
| XRCC5 | −0.41252298 | −0.42007243 |
| RTTN | −0.27426122 | −0.42003928 |
| AC091729.9 | −0.41998922 | −0.30812968 |
| ZFAND2A | −0.41998921 | −0.30812963 |
| FAM50B | −0.24568531 | −0.41974922 |
| ZCCHC24 | −0.3000804 | −0.41900341 |
| KIAA1161 | −0.41780651 | −0.31125164 |
| ORAI3 | −0.41675126 | −0.2260546 |
| RTKN | −0.37891422 | −0.41651837 |
| ARVCF | −0.4158601 | −0.33280454 |
| RANBP10 | −0.41582233 | −0.35862083 |
| GGCX | −0.41520765 | −0.4005541 |
| RAI14 | −0.28978227 | −0.41414754 |
| SEC24B | −0.41410303 | −0.40612039 |
| DHRS4-AS1 | −0.41357542 | −0.28741066 |
| HGD | −0.41354462 | −0.39801706 |
| AK2 | −0.33886441 | −0.41331637 |
| COMT | −0.41291208 | −0.40673668 |
| RP11-390F4.3 | −0.4128031 | −0.37317276 |
| IGSF8 | −0.41194622 | −0.35958714 |
| CNNM3 | −0.40992921 | −0.27139126 |
| CDA | −0.36907875 | −0.40982203 |
| NIPSNAP1 | −0.40921439 | −0.27157182 |
| MARC2 | −0.29767051 | −0.40886411 |
| RMDN2 | −0.33596855 | −0.40884522 |
| CDHR3 | −0.31811406 | −0.40828798 |
| CDKN2AIPNL | −0.35830649 | −0.40812345 |
| HMGCS2 | −0.35195098 | −0.40794621 |
| RUNDC3B | −0.40754375 | −0.36411206 |
| PAQR9 | −0.40581813 | −0.34342162 |
| PGM1 | −0.29171942 | −0.40564516 |
| CLU | −0.33992741 | −0.40451622 |
| METTL7B | −0.40440387 | −0.36579102 |
| OGFR-AS1 | −0.40303212 | −0.28290761 |
| OGFR | −0.40303194 | −0.28290762 |
| CNP | −0.40300593 | −0.27696223 |
| ALDH2 | −0.34376265 | −0.40235433 |
| SMARCD2 | −0.40191315 | −0.26016194 |
| SLC47A1 | −0.38887493 | −0.40078725 |
| MYH14 | −0.39185752 | −0.40078648 |
| SNTB1 | −0.29749725 | −0.39967707 |
| ZNF3 | −0.39956344 | −0.29301597 |
| PHYHD1 | −0.36517495 | −0.39823507 |
| CSAD | −0.39743692 | −0.31170411 |
| ACSL1 | −0.38375538 | −0.39712276 |
| CLDN15 | −0.2855084 | −0.39595715 |
| ZER1 | −0.39454397 | −0.33872364 |
| RP11-384L8.1 | −0.39319765 | −0.3070985 |
| CMTM8 | −0.39319761 | −0.30709859 |
| LRP1 | −0.29833253 | −0.39301776 |
| PKP2 | −0.34439876 | −0.3926357 |
| SLC25A27 | −0.33965267 | −0.39108688 |
| CYP39A1 | −0.33965247 | −0.39108607 |
| RNF220 | −0.36030039 | −0.39104117 |
| RP5-1033H22.2 | −0.30503081 | −0.39050236 |
| ECI2 | −0.39036176 | −0.30879026 |
| SIRT7 | −0.38972422 | −0.28326125 |
| FZD4 | −0.34499709 | −0.38933786 |
| SUV39H1 | −0.36971855 | −0.38888921 |
| TCF7L1 | −0.26986858 | −0.38839527 |
| CHST13 | −0.38814415 | −0.32402455 |
| NFIC | −0.38801358 | −0.31715258 |
| PC | −0.2713548 | −0.38793794 |
| SESN2 | −0.3709953 | −0.38782191 |
| AFMID | −0.38774641 | −0.30584992 |
| NR0B2 | −0.36539925 | −0.38774243 |
| SLC12A7 | −0.38663084 | −0.27527243 |
| CAPN5 | −0.34570202 | −0.38577686 |
| THRB-AS1 | −0.34943077 | −0.38575153 |
| ABHD6 | −0.34257166 | −0.38522473 |
| ATXN7L1 | −0.26845327 | −0.38521028 |
| TALDO1 | −0.38502538 | −0.30703897 |
| DGCR8 | −0.38471087 | −0.3773287 |
| ATP5SL | −0.31559414 | −0.38440531 |
| ZBTB42 | −0.38427243 | −0.2922977 |
| AKT1 | −0.38427243 | −0.29229771 |
| PANX2 | −0.38404028 | −0.36173835 |
| MTFR1 | −0.38367093 | −0.24254236 |
| SHMT1 | −0.38348468 | −0.35373178 |
| GCH1 | −0.2959687 | −0.38317213 |
| F12 | −0.38308371 | −0.27951505 |
| MTM1 | −0.38247333 | −0.29781583 |
| NME4 | −0.32377865 | −0.38216429 |
| SLCO2B1 | −0.38122455 | −0.26391129 |
| RASSF7 | −0.3811343 | −0.293724 |
| RP11-655M14.13 | −0.38109626 | −0.26735602 |
| TBX10 | −0.38109598 | −0.26735594 |
| NUDT8 | −0.38109581 | −0.2673559 |
| PSMA7 | −0.38098092 | −0.29845841 |
| TRMT2A | −0.38057839 | −0.22895339 |
| EEFSEC | −0.34037015 | −0.38025914 |
| HLF | −0.32957238 | −0.38009922 |
| PEX16 | −0.37993452 | −0.33492077 |
| PARP10 | −0.37981547 | −0.21387565 |
| RP11-134G8.7 | −0.27602285 | −0.37863625 |
| GRTP1 | −0.37814196 | −0.36530467 |
| HDLBP | −0.37797755 | −0.3024235 |
| MAVS | −0.37731114 | −0.36557947 |
| EPN1 | −0.37682644 | −0.27167571 |
| IRF2BP1 | −0.37674375 | −0.32099143 |
| ASB3 | −0.31361875 | −0.37647766 |
| TMEM80 | −0.37570265 | −0.24624656 |
| C16orf70 | −0.37492915 | −0.31083243 |
| SLC35D1 | −0.37436408 | −0.27595108 |
| SCAP | −0.37369073 | −0.28496227 |
| MDN1 | −0.3562244 | −0.3736243 |
| ADCK3 | −0.29812925 | −0.37307645 |
| HSD11B2 | −0.3726025 | −0.31254558 |
| PHYH | −0.3720753 | −0.33485628 |
| MMP15 | −0.35668513 | −0.37172165 |
| HOGA1 | −0.31742346 | −0.37107901 |
| RHCE | −0.37080779 | −0.30171794 |
| ARL6IP4 | −0.37076139 | −0.28891513 |
| AQP3 | −0.32343107 | −0.36985801 |
| LRR1 | −0.3287904 | −0.36848651 |
| SETD1A | −0.36744264 | −0.23242592 |
| TMEM57 | −0.3357718 | −0.36739329 |
| EPS8L2 | −0.36492322 | −0.22369652 |
| AP000355.2 | −0.36480429 | −0.31196501 |
| GPAM | −0.36443184 | −0.34811237 |
| FOXA3 | −0.36375897 | −0.29284569 |
| PIPOX | −0.36276665 | −0.28074058 |
| EPHB4 | −0.33746387 | −0.36213718 |
| TMEM192 | −0.27675938 | −0.36162899 |
| ANG | −0.25749011 | −0.36109385 |
| SMPDL3A | −0.25931705 | −0.36022303 |
| NT5DC3 | −0.26949837 | −0.35751769 |
| WDR81 | −0.35673733 | −0.25774597 |
| RAP2C | −0.35660216 | −0.24521162 |
| RP11-350G8.5 | −0.3543378 | −0.35485534 |
| EPB41L5 | −0.35472404 | −0.2812191 |
| CERS2 | −0.26745357 | −0.35404273 |
| GTF2IRD1 | −0.35349659 | −0.33576831 |
| PEX19 | −0.35337264 | −0.34915125 |
| AKAP1 | −0.35263918 | −0.29265316 |
| SH2D4A | −0.25490705 | −0.35212954 |
| NR1H3 | −0.35210006 | −0.27146195 |
| PDIK1L | −0.35204896 | −0.2829924 |
| ANXA9 | −0.26745198 | −0.35124338 |
| PFKFB1 | −0.33473836 | −0.35099402 |
| KANK2 | −0.35036754 | −0.3319646 |
| SHH | −0.32551636 | −0.35032606 |
| TMEM101 | −0.33732052 | −0.34983763 |
| RXRA | −0.34948675 | −0.28767448 |
| HPCAL1 | −0.28039765 | −0.34899215 |
| C1RL-AS1 | −0.34870299 | −0.3393696 |
| KIF1C | −0.34811672 | −0.23372311 |
| IDNK | −0.2652978 | −0.34799289 |
| SIVA1 | −0.34716386 | −0.27226572 |
| ELK1 | −0.34650465 | −0.30471213 |
| IL6R | −0.34129699 | −0.3463338 |
| NEIL1 | −0.34551526 | −0.32270888 |
| SERPINF1 | −0.34534084 | −0.33522948 |
| FCGRT | −0.34444097 | −0.27204935 |
| AGFG2 | −0.34421296 | −0.29824866 |
| MUM1 | −0.34369254 | −0.32853246 |
| ABHD14A | −0.34363545 | −0.23778997 |
| CLASRP | −0.34316616 | −0.25338292 |
| MLX | −0.34239925 | −0.32670861 |
| ACY1 | −0.34232231 | −0.247427 |
| STARD5 | −0.3419298 | −0.31524065 |
| FZD5 | −0.25822211 | −0.3409676 |
| DCAF8 | −0.27681872 | −0.339734 |
| ZNF408 | −0.26236338 | −0.33957453 |
| ALG3 | −0.3389394 | −0.32429392 |
| WIZ | −0.3386708 | −0.23583346 |
| BAIAP2L1 | −0.26230457 | −0.33833424 |
| CTIF | −0.32081451 | −0.33504599 |
| MACROD1 | −0.33385226 | −0.31944701 |
| WWC2-AS2 | −0.22097621 | −0.3336651 |
| COASY | −0.33200618 | −0.31079482 |
| GLTPD2 | −0.33175708 | −0.27314765 |
| SEPHS2 | −0.27031363 | −0.3309181 |
| CES2 | −0.32990787 | −0.24570301 |
| FAM96B | −0.32990787 | −0.24570233 |
| SDC1 | −0.26936324 | −0.32918957 |
| ZBTB39 | −0.32865224 | −0.32095137 |
| HDAC4 | −0.32840301 | −0.31694505 |
| SLC23A3 | −0.32771937 | −0.31415409 |
| XBP1 | −0.23846671 | −0.32709786 |
| C1orf220 | −0.32669137 | −0.27036106 |
| AP000253.1 | −0.32451248 | −0.29401886 |
| SLC35A3 | −0.29282936 | −0.32197216 |
| PPP1R26 | −0.3197786 | −0.29937873 |
| PCTP | −0.28365657 | −0.3197411 |
| RPS29 | −0.27154984 | −0.319368 |
| LTB4R2 | −0.27227953 | −0.31874686 |
| LTB4R | −0.27227964 | −0.31874682 |
| KIAA2013 | −0.31837726 | −0.27583915 |
| SOD1 | −0.31814476 | −0.30820525 |
| NFYC | −0.31229081 | −0.31672619 |
| DDI2 | −0.31641086 | −0.26843984 |
| PLA2G12B | −0.31627894 | −0.27972574 |
| GSDMD | −0.31549845 | −0.28092864 |
| C2 | −0.31260761 | −0.31434771 |
| ENKD1 | −0.31330502 | −0.30424951 |
| SUN2 | −0.31313228 | −0.23607547 |
| NUDT7 | −0.29642592 | −0.31281805 |
| CDON | −0.31268371 | −0.26055483 |
| PEX11G | −0.28673975 | −0.31256604 |
| UPB1 | −0.31139815 | −0.27512159 |
| COBL | −0.31078456 | −0.27482485 |
| CTNNBIP1 | −0.31070486 | −0.26971012 |
| FAHD2A | −0.2759954 | −0.31009587 |
| RAB40B | −0.27038904 | −0.30958601 |
| NAPA | −0.30932566 | −0.24080098 |
| ATP11C | −0.30919624 | −0.29446512 |
| RBL2 | −0.30816824 | −0.25066095 |
| TPCN1 | −0.2009117 | −0.30642959 |
| KLHDC2 | −0.25686425 | −0.30247648 |
| MCEE | −0.30024145 | −0.29215766 |
| SLC37A4 | −0.27316758 | −0.30000485 |
| VAMP8 | −0.29952151 | −0.27377672 |
| SLC25A44 | −0.29879377 | −0.29111812 |
| AKAP8 | −0.29806694 | −0.27827847 |
| ERMARD | −0.29347634 | −0.29781332 |
| NELFB | −0.29745893 | −0.24232981 |
| GRINA | −0.29608637 | −0.22354455 |
| FAM35A | −0.29523254 | −0.26738233 |
| RPL41 | −0.29310028 | −0.23916321 |
| ZNF490 | −0.24853924 | −0.29208614 |
| RP4-605O3.4 | −0.2906624 | −0.23350988 |
| COX14 | −0.2906624 | −0.2335097 |
| PMF1 | −0.22145396 | −0.28935479 |
| DNAJC25 | −0.2511999 | −0.28893162 |
| MTMR4 | −0.28516127 | −0.28642688 |
| CRTC2 | −0.25459398 | −0.28468128 |
| MCCC2 | −0.28210687 | −0.26364444 |
| ZNF787 | −0.27950087 | −0.21988141 |
| NPRL3 | −0.25928478 | −0.27888637 |
| RAB11FIP3 | −0.25547192 | −0.27748997 |
| PLXNB2 | −0.2757926 | −0.20709931 |
| RP11-1055B8.4 | −0.24897767 | −0.27466938 |
| TPRN | −0.25973517 | −0.27431518 |
| DBI | −0.23974492 | −0.26067366 |
| ABCB6 | −0.25566735 | −0.25991797 |
| COG8 | −0.25807848 | −0.23033772 |
| SLC27A3 | −0.22292407 | −0.25561704 |
| RP11-1275H24.1 | −0.22375083 | −0.25122346 |
| RP11-1275H24.3 | −0.22375074 | −0.25122324 |
| ZNF689 | −0.24985306 | −0.24292946 |
| ATXN2 | −0.23871843 | −0.24756363 |
| OGG1 | −0.23787695 | −0.24669267 |
| ILK | 0.2107524 | 0.21567035 |
| HIPK1 | 0.22308137 | 0.22679421 |
| AFTPH | 0.22903256 | 0.23199635 |
| EZR | 0.26048853 | 0.25396407 |
| ATM | 0.25494652 | 0.26255025 |
| PLEKHA2 | 0.2728107 | 0.25542597 |
| DCHS1 | 0.27428179 | 0.24121081 |
| C15orf39 | 0.27940961 | 0.25906811 |
| GAL3ST4 | 0.24442711 | 0.28107799 |
| CASP8 | 0.28831686 | 0.28884766 |
| GGPS1 | 0.27350961 | 0.2914372 |
| PPARD | 0.29422585 | 0.26209515 |
| PRPF38B | 0.28612183 | 0.29435086 |
| HIVEP2 | 0.29979044 | 0.26748027 |
| DENND3 | 0.26159665 | 0.30049974 |
| ADD3 | 0.30430185 | 0.28750897 |
| RP11-356I2.4 | 0.27518333 | 0.30451362 |
| CTC1 | 0.27179002 | 0.30572551 |
| CREB1 | 0.30337455 | 0.30671961 |
| SSH2 | 0.30718237 | 0.28522004 |
| MAP3K1 | 0.26723477 | 0.3077578 |
| CACUL1 | 0.3085782 | 0.2941355 |
| FGD6 | 0.3089605 | 0.23620758 |
| OSTM1 | 0.29658056 | 0.31139925 |
| NABP1 | 0.31071937 | 0.31259699 |
| SLC43A2 | 0.28064101 | 0.31293773 |
| TUBA1C | 0.29126926 | 0.31379851 |
| GNA13 | 0.31531003 | 0.26145623 |
| SPAG4 | 0.31660075 | 0.31274341 |
| PARP8 | 0.28475341 | 0.317782 |
| SYT11 | 0.32092233 | 0.32197549 |
| GON4L | 0.3209226 | 0.32197557 |
| RAB34 | 0.28369033 | 0.32260509 |
| ARL4A | 0.323018 | 0.3129778 |
| LEPROTL1 | 0.32585751 | 0.27928831 |
| YWHAH | 0.32625776 | 0.31678903 |
| HIVEP3 | 0.29366104 | 0.32863623 |
| CHMP4A | 0.25714653 | 0.32963386 |
| ARPC5 | 0.32536411 | 0.33003866 |
| ANXA11 | 0.33060593 | 0.2010745 |
| OGFRL1 | 0.33488857 | 0.30733855 |
| KLHL5 | 0.3353751 | 0.28553148 |
| IGF1R | 0.34046751 | 0.32825058 |
| COL4A2 | 0.34215423 | 0.25903599 |
| PAK1 | 0.3427117 | 0.32645573 |
| JUN | 0.34682145 | 0.29406581 |
| MTMR11 | 0.34716135 | 0.28582582 |
| LINC00857 | 0.34786949 | 0.22936654 |
| PDCD4 | 0.26620003 | 0.35243856 |
| ANKRD53 | 0.31397449 | 0.3549392 |
| FHL3 | 0.3431548 | 0.35544121 |
| CD63 | 0.3167874 | 0.35658012 |
| F2R | 0.35693987 | 0.35232893 |
| MAP3K8 | 0.35769287 | 0.27357984 |
| SERPINB9 | 0.31953944 | 0.3598179 |
| PRDM2 | 0.28054952 | 0.35994273 |
| PRKCH | 0.36102968 | 0.27743854 |
| IFFO2 | 0.36126927 | 0.34581623 |
| ZNF160 | 0.35993642 | 0.36378827 |
| ZNF397 | 0.31489028 | 0.36584279 |
| TCP11L2 | 0.28389699 | 0.36684095 |
| ACER2 | 0.36782935 | 0.30536002 |
| U73166.2 | 0.36816264 | 0.32979711 |
| GPRIN3 | 0.35076587 | 0.36842184 |
| SERAC1 | 0.36849956 | 0.29506972 |
| RNF145 | 0.36582755 | 0.36903238 |
| SH3RF3 | 0.2870755 | 0.36954186 |
| CTD-2201E18.3 | 0.36995465 | 0.33561194 |
| SMURF2 | 0.30973965 | 0.37256902 |
| CD47 | 0.373919 | 0.36315117 |
| MKL1 | 0.37531047 | 0.29102788 |
| MAPRE1 | 0.35081922 | 0.37557016 |
| BCL2 | 0.3620567 | 0.37762952 |
| ARHGEF2 | 0.2802985 | 0.37847804 |
| TSPYL1 | 0.37911655 | 0.27570659 |
| RALGDS | 0.31588532 | 0.38045161 |
| USP37 | 0.38160616 | 0.34059824 |
| GNA12 | 0.38340653 | 0.29021981 |
| RUNX2 | 0.29206024 | 0.38402238 |
| IQGAP1 | 0.3841836 | 0.37678882 |
| CDC42EP3 | 0.38492335 | 0.37741779 |
| CCNG2 | 0.34678301 | 0.38542955 |
| PAFAH1B2 | 0.38586348 | 0.35567709 |
| DUSP5 | 0.37846408 | 0.38624474 |
| RNF24 | 0.38748492 | 0.29782783 |
| PLA2G4C | 0.38868241 | 0.37218793 |
| ITGA6 | 0.28413376 | 0.3897485 |
| SLC12A2 | 0.3912081 | 0.36340859 |
| GYPC | 0.30513307 | 0.39166292 |
| IL2RA | 0.39535583 | 0.32875042 |
| SLC25A12 | 0.39580534 | 0.23786379 |
| ZNF304 | 0.32302879 | 0.39613419 |
| RAP2B | 0.37400913 | 0.39725167 |
| FAM129B | 0.36540402 | 0.39745065 |
| CAPN2 | 0.38426856 | 0.39788697 |
| AAGAB | 0.39411787 | 0.4028782 |
| KIAA1462 | 0.40346982 | 0.39022757 |
| SNN | 0.29266211 | 0.40383775 |
| CTB-55O6.12 | 0.24814694 | 0.40494538 |
| MDFIC | 0.40499853 | 0.31179508 |
| IGFBP7 | 0.37855108 | 0.40553844 |
| PLCD3 | 0.30730081 | 0.40569611 |
| NOD2 | 0.4063656 | 0.31642647 |
| STK17A | 0.3926019 | 0.40642822 |
| POU6F1 | 0.38276416 | 0.40795243 |
| CEP135 | 0.40805415 | 0.34136025 |
| ZNF829 | 0.35517015 | 0.40811835 |
| AEN | 0.38432407 | 0.40843242 |
| ZNF568 | 0.33969794 | 0.40852172 |
| TTC16 | 0.40948024 | 0.39201105 |
| IDS | 0.37552294 | 0.40957342 |
| HSPG2 | 0.3918783 | 0.41001784 |
| ATP2B4 | 0.26574319 | 0.41007285 |
| HELB | 0.41035191 | 0.40435962 |
| FAM49B | 0.29091936 | 0.41106319 |
| AGO2 | 0.28394163 | 0.41116859 |
| SYNGAP1 | 0.28654513 | 0.41198544 |
| CDC42SE2 | 0.41240258 | 0.3792452 |
| LY6G5C | 0.38755945 | 0.41256565 |
| SP110 | 0.35181251 | 0.4129386 |
| REL | 0.41352048 | 0.27269062 |
| FAM228B | 0.41355544 | 0.39511166 |
| LUZP1 | 0.41366119 | 0.3241621 |
| CD58 | 0.37465017 | 0.41459738 |
| TULP3 | 0.41536548 | 0.39175604 |
| JAG1 | 0.41536706 | 0.27433069 |
| ZNF292 | 0.41604909 | 0.30230274 |
| CTTNBP2NL | 0.41729261 | 0.30793738 |
| CCNJ | 0.36735682 | 0.41765825 |
| CCDC28B | 0.340358 | 0.41972829 |
| ZNF512 | 0.34494758 | 0.41998657 |
| BBS7 | 0.38128807 | 0.42024751 |
| HSPA12A | 0.42119491 | 0.35460429 |
| PRICKLE2 | 0.4223884 | 0.2695499 |
| RAVER2 | 0.42464549 | 0.39642982 |
| GRIN2D | 0.3772624 | 0.42483366 |
| EGLN3 | 0.35420715 | 0.42732412 |
| ZNF614 | 0.42734074 | 0.32668887 |
| TIMP2 | 0.42790695 | 0.3976555 |
| MXD1 | 0.42906983 | 0.33033446 |
| PKDCC | 0.36179776 | 0.42972519 |
| SFMBT2 | 0.34457148 | 0.43061116 |
| PLP2 | 0.43116284 | 0.37154635 |
| DYRK2 | 0.34939968 | 0.43140284 |
| LOXL3 | 0.33730819 | 0.43211472 |
| DOK1 | 0.33730827 | 0.43211541 |
| TMSB10 | 0.34648728 | 0.43295041 |
| EVA1C | 0.43400964 | 0.4169852 |
| SYNJ2 | 0.43404459 | 0.37729678 |
| DNAJC10 | 0.41325387 | 0.43449638 |
| FZD3 | 0.43463077 | 0.36760367 |
| KIRREL | 0.4351682 | 0.41940162 |
| RPS6KA3 | 0.43588415 | 0.29224785 |
| ZNF347 | 0.43610448 | 0.43305458 |
| HSPBAP1 | 0.44034637 | 0.26476308 |
| TP53I3 | 0.43168036 | 0.44063386 |
| ANKRD44 | 0.35421672 | 0.44114336 |
| STK4 | 0.30557742 | 0.44162894 |
| PHACTR2 | 0.44186311 | 0.30109068 |
| RP1-111C20.4 | 0.29870027 | 0.44232855 |
| HMGN4 | 0.39458524 | 0.44312601 |
| ZNF548 | 0.29203637 | 0.44325844 |
| ETS1 | 0.38507175 | 0.44343685 |
| PDE7A | 0.44419666 | 0.42157149 |
| C1QTNF1 | 0.44015928 | 0.44453413 |
| NF1 | 0.44460792 | 0.34325888 |
| PMP22 | 0.44598518 | 0.4028151 |
| TAGLN2 | 0.41063153 | 0.44666745 |
| ANTXR1 | 0.37101085 | 0.44800178 |
| NXPE3 | 0.44813856 | 0.27467834 |
| UBASH3B | 0.35945585 | 0.44966858 |
| SLC41A1 | 0.44969093 | 0.42095754 |
| TMEM217 | 0.44969829 | 0.31413917 |
| PPP1R12B | 0.45108735 | 0.26002915 |
| PAG1 | 0.31164018 | 0.45144144 |
| LINC00092 | 0.33776175 | 0.45248883 |
| ANKRD36C | 0.42114693 | 0.45312528 |
| SLC6A9 | 0.38816511 | 0.45316169 |
| QSOX1 | 0.42971667 | 0.45443878 |
| HMGCR | 0.45463584 | 0.33868725 |
| PAPOLG | 0.45494359 | 0.42735562 |
| MPP3 | 0.45501537 | 0.42775513 |
| AMIGO2 | 0.37551093 | 0.45574505 |
| PDE4B | 0.45635271 | 0.33535628 |
| TMEM185B | 0.40094285 | 0.45663537 |
| ALOX5 | 0.31486994 | 0.45679653 |
| PRKX | 0.45686789 | 0.45514033 |
| C10orf128 | 0.38293117 | 0.45754484 |
| DDHD1 | 0.45794778 | 0.37834292 |
| RFX5 | 0.35284567 | 0.45828102 |
| PAM | 0.45896442 | 0.41257519 |
| ARMC2 | 0.45969429 | 0.25523299 |
| EMP3 | 0.30966654 | 0.46017564 |
| PRTFDC1 | 0.43036982 | 0.46088073 |
| NFKBIE | 0.25910283 | 0.46144042 |
| KIAA0556 | 0.34073991 | 0.46244078 |
| WIPF1 | 0.34676681 | 0.46304747 |
| UBE2Q2 | 0.46342887 | 0.44039956 |
| DNM3 | 0.46708241 | 0.4226569 |
| PACS1 | 0.37466034 | 0.46757148 |
| STARD3NL | 0.46838676 | 0.42147856 |
| ABR | 0.31153821 | 0.46976107 |
| MAFK | 0.47102533 | 0.40103476 |
| EML2 | 0.45565411 | 0.4724727 |
| HECA | 0.32915436 | 0.47786029 |
| FHOD1 | 0.40956315 | 0.47825439 |
| ZNF611 | 0.39414727 | 0.47916196 |
| FAM188B | 0.47938055 | 0.35957877 |
| FKBP10 | 0.45869871 | 0.47991687 |
| CEP85L | 0.48009881 | 0.3076561 |
| CAV2 | 0.48010438 | 0.2827198 |
| PRDM1 | 0.46971767 | 0.48160546 |
| TMEM184B | 0.48163047 | 0.47869769 |
| IER5 | 0.37380049 | 0.48238365 |
| GPRASP1 | 0.38849838 | 0.48242602 |
| ANK1 | 0.33321029 | 0.48282041 |
| GAS8 | 0.43469481 | 0.48323083 |
| HIST1H4H | 0.41684649 | 0.48488746 |
| PIP4K2A | 0.40369193 | 0.48490063 |
| FAM57A | 0.42520587 | 0.4849433 |
| ARF3 | 0.40778092 | 0.4854765 |
| STXBP1 | 0.48770991 | 0.46077966 |
| SYTL3 | 0.23936244 | 0.48812979 |
| SCLT1 | 0.48933128 | 0.42659741 |
| TPM2 | 0.49037062 | 0.4880653 |
| ARRDC4 | 0.34973272 | 0.49054962 |
| RUNX1 | 0.4910348 | 0.39624144 |
| LHFPL2 | 0.49123161 | 0.34682454 |
| CUEDC1 | 0.49161992 | 0.33336144 |
| SEL1L3 | 0.41317706 | 0.49164071 |
| ZC3HAVIL | 0.38668801 | 0.49225992 |
| DACT1 | 0.49339673 | 0.45229507 |
| ANK3 | 0.3784585 | 0.49503629 |
| BIRC3 | 0.483975 | 0.49518394 |
| APH1B | 0.49565719 | 0.46748055 |
| PDE4D | 0.4965212 | 0.36657125 |
| CMTM7 | 0.33828934 | 0.49655722 |
| TYROBP | 0.30310964 | 0.496969 |
| GLIPR2 | 0.30316371 | 0.4971785 |
| IKZF2 | 0.49752303 | 0.27186267 |
| CACNB3 | 0.44463524 | 0.49869309 |
| ARHGAP27 | 0.30243399 | 0.49974341 |
| MMP25 | 0.36376234 | 0.50007319 |
| ABCC4 | 0.50075499 | 0.3744854 |
| PPP1R2 | 0.30381138 | 0.50101244 |
| PHLDB1 | 0.39787274 | 0.5012486 |
| MEF2C | 0.50294246 | 0.35897157 |
| CEP97 | 0.50332863 | 0.4070238 |
| ITPKB | 0.33312205 | 0.50451849 |
| METRNL | 0.32182726 | 0.50659894 |
| COL4A1 | 0.50693035 | 0.45289629 |
| DBN1 | 0.46345206 | 0.50779986 |
| TNFAIP8 | 0.41689345 | 0.50951227 |
| CHST15 | 0.50987762 | 0.43353198 |
| HID1 | 0.44648635 | 0.51027467 |
| FBLIM1 | 0.51097315 | 0.44851917 |
| PCSK5 | 0.51289366 | 0.40155007 |
| ARRDC2 | 0.43813799 | 0.51427321 |
| MFSD7 | 0.32302576 | 0.51444871 |
| SMARCD3 | 0.37790608 | 0.51456669 |
| MMP14 | 0.51457997 | 0.35766629 |
| STK32C | 0.29817777 | 0.51482284 |
| BARD1 | 0.5157784 | 0.49116312 |
| ARHGEF19 | 0.34717626 | 0.51597494 |
| RP11-344B5.2 | 0.47688681 | 0.5160077 |
| GATA2 | 0.40734865 | 0.51642578 |
| FAM102B | 0.51018536 | 0.51646631 |
| PHF20 | 0.35967186 | 0.51714878 |
| FOS | 0.51723482 | 0.49843324 |
| PLAGL1 | 0.36332848 | 0.51831473 |
| ADAMTS10 | 0.51891303 | 0.45949198 |
| VCL | 0.51963989 | 0.3093319 |
| NFKB2 | 0.3763195 | 0.52061422 |
| ZNF267 | 0.52210061 | 0.40389285 |
| ZNF737 | 0.52232205 | 0.50969225 |
| ARHGDIB | 0.42518457 | 0.52245756 |
| ZNF85 | 0.49766617 | 0.52266336 |
| GEM | 0.49369967 | 0.52275169 |
| SH3YL1 | 0.52462088 | 0.39617475 |
| TP53BP1 | 0.41798994 | 0.52582135 |
| CHST3 | 0.52597136 | 0.39870375 |
| MEF2C-AS1 | 0.52831047 | 0.43584248 |
| BTN2A3P | 0.45618558 | 0.52891207 |
| RP11-10K16.1 | 0.47912049 | 0.52942448 |
| GALNT7 | 0.4791205 | 0.52942741 |
| PPP1R15A | 0.51438974 | 0.52960335 |
| CASP1 | 0.53004311 | 0.38226252 |
| FAM131A | 0.53052353 | 0.47029867 |
| CDKN2A | 0.53121383 | 0.49892258 |
| CHST11 | 0.35013813 | 0.53121634 |
| NEURL3 | 0.53200576 | 0.40525289 |
| SPINT2 | 0.40865108 | 0.53216305 |
| RIN1 | 0.50104036 | 0.53266083 |
| ANKDD1B | 0.53338926 | 0.36210881 |
| CDKN1A | 0.53585024 | 0.37035565 |
| PAQR8 | 0.42941828 | 0.53590815 |
| RASGRP3 | 0.53609675 | 0.42685963 |
| PDE3A | 0.53625641 | 0.41263336 |
| PDE5A | 0.53732958 | 0.45986461 |
| DNAJB6 | 0.43164569 | 0.5373962 |
| GIPR | 0.53501251 | 0.53901154 |
| NKRF | 0.53907752 | 0.48815413 |
| TNFRSF12A | 0.54017298 | 0.47555485 |
| B3GNT7 | 0.40492137 | 0.5431056 |
| SLC1A5 | 0.41749864 | 0.54348945 |
| NOTCH3 | 0.54369334 | 0.3879665 |
| HCG11 | 0.35711029 | 0.54571694 |
| AC092835.2 | 0.36293675 | 0.54587032 |
| HDAC7 | 0.4644996 | 0.54608113 |
| ARL4C | 0.38595878 | 0.54751588 |
| RPS6KA1 | 0.2448949 | 0.54783821 |
| SLC26A2 | 0.47719781 | 0.5479984 |
| E2F3 | 0.54912729 | 0.4093195 |
| C10orf54 | 0.27096674 | 0.55216616 |
| NFKBID | 0.35908523 | 0.55219818 |
| HCST | 0.35908535 | 0.5521982 |
| ENDOD1 | 0.55248025 | 0.55221703 |
| IMPDH1 | 0.40902717 | 0.55270406 |
| ANXA2R | 0.55341079 | 0.52331638 |
| AC025171.1 | 0.55341079 | 0.52331644 |
| PEA15 | 0.55550754 | 0.54654346 |
| TRABD2A | 0.34077103 | 0.55605791 |
| FLVCR1 | 0.5560836 | 0.33767305 |
| TUBB6 | 0.55625243 | 0.49154716 |
| CHORDC1 | 0.5564496 | 0.54151163 |
| CAV1 | 0.55749876 | 0.30249684 |
| SLC35F2 | 0.55860647 | 0.51792416 |
| TNFSF8 | 0.51293016 | 0.55941066 |
| PDP1 | 0.39213174 | 0.55993107 |
| NRSN2 | 0.56069089 | 0.56040862 |
| FAM167A | 0.4562808 | 0.56243388 |
| FMNL3 | 0.36738023 | 0.56260499 |
| PHF19 | 0.30788195 | 0.56263611 |
| AP3M2 | 0.31214264 | 0.56298546 |
| MTHFD1L | 0.563114 | 0.3889355 |
| ITPR3 | 0.34723006 | 0.56319202 |
| SYK | 0.56337245 | 0.54784322 |
| ISG20 | 0.45028012 | 0.56611837 |
| TRIM31 | 0.51590358 | 0.56710758 |
| RARG | 0.38131729 | 0.56736657 |
| TMEM51 | 0.56747986 | 0.42353012 |
| RP11-712B9.2 | 0.51234728 | 0.5677274 |
| PCBP3 | 0.45831443 | 0.56812112 |
| ARHGAP31 | 0.56256999 | 0.56887828 |
| INPP4B | 0.50195657 | 0.56989785 |
| PRICKLE1 | 0.57026461 | 0.5689147 |
| SWAP70 | 0.57121006 | 0.54702142 |
| TMEM173 | 0.31375224 | 0.5719445 |
| ALOX5AP | 0.47668661 | 0.57400025 |
| WNT4 | 0.44679828 | 0.57537098 |
| C5orf30 | 0.57591781 | 0.5074811 |
| MARCH3 | 0.57605523 | 0.45001766 |
| PWWP2B | 0.30247751 | 0.57642316 |
| PTPRH | 0.5767047 | 0.53219812 |
| TMEM51-AS1 | 0.57689266 | 0.42152941 |
| HNRNPA1L2 | 0.5668362 | 0.57929393 |
| DBNDD1 | 0.55159325 | 0.57950392 |
| ADAM9 | 0.5809513 | 0.27644204 |
| AP1S3 | 0.58125243 | 0.4899579 |
| GSTM5 | 0.53508569 | 0.58281795 |
| GSN | 0.5830383 | 0.34920528 |
| PLAU | 0.36932798 | 0.58416417 |
| RAB8B | 0.5843851 | 0.43509498 |
| ZNF486 | 0.51365374 | 0.58467117 |
| CHD3 | 0.34974385 | 0.5848934 |
| RP11-848P1.9 | 0.57955488 | 0.58526065 |
| ZNF853 | 0.4073588 | 0.58652254 |
| DLG3 | 0.52896732 | 0.58685481 |
| FGFR1 | 0.58780905 | 0.51033287 |
| STX11 | 0.58956956 | 0.55775123 |
| ZSWIM4 | 0.58973443 | 0.56286966 |
| CD83 | 0.43427826 | 0.59034087 |
| P2RX1 | 0.39595553 | 0.59207479 |
| SYCP2 | 0.50774449 | 0.59353471 |
| PHLDA3 | 0.59361913 | 0.48179321 |
| SLC25A24 | 0.59527835 | 0.45030916 |
| FAR1 | 0.37933675 | 0.59574564 |
| ITGA3 | 0.59653283 | 0.57519978 |
| IL34 | 0.52681978 | 0.59673106 |
| PPDPF | 0.37317828 | 0.59688014 |
| COL16A1 | 0.53911551 | 0.59846358 |
| FUT4 | 0.52582932 | 0.59851705 |
| ZDHHC1 | 0.59939596 | 0.49127992 |
| TPM4 | 0.42021788 | 0.59985211 |
| MCAM | 0.60098824 | 0.5073176 |
| STAT4 | 0.60106806 | 0.5689905 |
| FBLN5 | 0.41215194 | 0.60110259 |
| HMGA1 | 0.4465091 | 0.60122377 |
| ZSCAN9 | 0.60170843 | 0.45016034 |
| LOXL1 | 0.39225189 | 0.60176019 |
| ENAH | 0.60282452 | 0.37060372 |
| IFI27L2 | 0.58017434 | 0.60293347 |
| KIF3C | 0.54475307 | 0.60434703 |
| EIF5A2 | 0.58321585 | 0.60488452 |
| F3 | 0.6056885 | 0.49185377 |
| CD96 | 0.56613089 | 0.6062371 |
| TRNP1 | 0.60679965 | 0.3961341 |
| ITM2C | 0.47444143 | 0.60811754 |
| CLSTN1 | 0.52612578 | 0.60983087 |
| MTHFD2 | 0.49159257 | 0.6101798 |
| LINC00271 | 0.6117606 | 0.36538242 |
| AHI1 | 0.61176064 | 0.36538254 |
| PTGER4 | 0.3781944 | 0.61257286 |
| SRGN | 0.61265101 | 0.56588221 |
| PARP6 | 0.40968134 | 0.61266417 |
| C1orf116 | 0.6127681 | 0.48258022 |
| CYS1 | 0.61404874 | 0.58710389 |
| ZNF816 | 0.49012137 | 0.61509152 |
| ATP9A | 0.61548665 | 0.31186667 |
| CYTIP | 0.53009704 | 0.61590384 |
| C1orf145 | 0.42581101 | 0.61601003 |
| COL12A1 | 0.61787272 | 0.55428585 |
| SESN3 | 0.41189014 | 0.6196084 |
| ALDH1A3 | 0.62036516 | 0.54893551 |
| PDLIM4 | 0.62041818 | 0.52071777 |
| ABCC1 | 0.6022527 | 0.62140983 |
| ARSJ | 0.6218029 | 0.38518403 |
| RELB | 0.40104726 | 0.62198863 |
| MYOF | 0.62355697 | 0.47967252 |
| GSTP1 | 0.56525828 | 0.62403088 |
| RHOQ | 0.62453525 | 0.50619135 |
| LRP8 | 0.44236347 | 0.62479346 |
| DAGLA | 0.59829134 | 0.62492443 |
| LTBP4 | 0.35542217 | 0.62495215 |
| BATF3 | 0.62535223 | 0.60967772 |
| TEAD4 | 0.62582399 | 0.43564597 |
| TP53I11 | 0.59236895 | 0.62614093 |
| COL1A2 | 0.47829077 | 0.62628384 |
| RIMKLB | 0.62717825 | 0.45935488 |
| RIPK3 | 0.49096529 | 0.6277245 |
| MYO5A | 0.40920988 | 0.62779222 |
| PYGO1 | 0.45202523 | 0.62840257 |
| ROBO1 | 0.62849096 | 0.55898068 |
| ZNF529 | 0.55712458 | 0.62858898 |
| RAB11FIP5 | 0.62919558 | 0.36616513 |
| RECK | 0.6294769 | 0.43788935 |
| BEX4 | 0.5910851 | 0.63005211 |
| SFXN3 | 0.58797575 | 0.63040854 |
| PIWIL4 | 0.6313442 | 0.5386261 |
| SPECC1 | 0.47948072 | 0.63444676 |
| PAQR5 | 0.63467314 | 0.53113008 |
| PLEKHG4 | 0.38365905 | 0.63495302 |
| PRR7 | 0.4790133 | 0.6349686 |
| NMNAT2 | 0.63588177 | 0.63561552 |
| ISYNA1 | 0.43951788 | 0.6369664 |
| VIM-AS1 | 0.51139609 | 0.63730363 |
| LINC00982 | 0.60717294 | 0.63741163 |
| GBGT1 | 0.41594828 | 0.63834565 |
| ZNF667-AS1 | 0.52877945 | 0.63838193 |
| ZNF667 | 0.52877916 | 0.63838209 |
| RAB11FIP1 | 0.38599858 | 0.63846934 |
| ODF2L | 0.63858194 | 0.60858386 |
| NCK2 | 0.51803331 | 0.63867034 |
| DZIP1L | 0.63933721 | 0.60065084 |
| SLC38A1 | 0.5888489 | 0.63944904 |
| AKR1B1 | 0.56929296 | 0.64032396 |
| SCRN1 | 0.64171508 | 0.56966255 |
| HENMT1 | 0.41349065 | 0.64282589 |
| SLC45A4 | 0.48788347 | 0.64300174 |
| VIM | 0.50548708 | 0.64433933 |
| ZNF506 | 0.64461734 | 0.61013154 |
| SLC4A8 | 0.42042218 | 0.6446396 |
| OSBPL7 | 0.45545053 | 0.64485672 |
| MLLT3 | 0.59936912 | 0.64569286 |
| AC144831.1 | 0.58979539 | 0.64742621 |
| RP11-353N14.5 | 0.53817712 | 0.64832393 |
| TMEM243 | 0.49426528 | 0.64920592 |
| PLD4 | 0.50270134 | 0.64991953 |
| SLC7A6 | 0.41512372 | 0.65110127 |
| FRAS1 | 0.65177099 | 0.53817222 |
| XYLT1 | 0.42788894 | 0.65179297 |
| CHIT1 | 0.39041954 | 0.6519958 |
| DNAJC6 | 0.65338665 | 0.56617878 |
| BICD1 | 0.65364578 | 0.56603614 |
| CYR61 | 0.65428371 | 0.47510669 |
| ZNF426 | 0.65441711 | 0.43673876 |
| OBSCN | 0.40311877 | 0.65513282 |
| CLCF1 | 0.6554957 | 0.62940671 |
| S100A11 | 0.65702966 | 0.60215854 |
| SLC6A6 | 0.44054689 | 0.65770945 |
| CARD16 | 0.65908174 | 0.58386911 |
| C7orf31 | 0.40684957 | 0.66003683 |
| LCA5 | 0.6600795 | 0.43118261 |
| ZFP82 | 0.62599046 | 0.66067734 |
| LXN | 0.66111868 | 0.48350004 |
| KIAA0226L | 0.50874131 | 0.66190765 |
| LBH | 0.34498832 | 0.66191651 |
| CD59 | 0.66292552 | 0.3725589 |
| RP11-44N21.1 | 0.50320866 | 0.66316455 |
| BCL11A | 0.56981758 | 0.66319515 |
| ATP6V1E2 | 0.66322131 | 0.52488336 |
| SOD3 | 0.58411381 | 0.66330532 |
| ABCA7 | 0.27373557 | 0.66455718 |
| VANGL2 | 0.66497775 | 0.63181702 |
| MAP2 | 0.66574025 | 0.4419067 |
| TUBA1A | 0.53760031 | 0.66603317 |
| FLRT2 | 0.66632757 | 0.55087694 |
| IFT57 | 0.66668838 | 0.53532668 |
| AC006129.2 | 0.40575517 | 0.66707076 |
| RP11-1149O23.3 | 0.53127556 | 0.66905934 |
| NFATC4 | 0.56365233 | 0.6691852 |
| ADRA2A | 0.66896096 | 0.67013393 |
| KIAA1549 | 0.67077788 | 0.3846937 |
| RAP1GAP2 | 0.67059011 | 0.67200911 |
| PKM | 0.645632 | 0.67446074 |
| HLA-L | 0.53280219 | 0.67606992 |
| CD44 | 0.41471387 | 0.67718683 |
| ORAI2 | 0.40671418 | 0.678431 |
| STK39 | 0.55501553 | 0.67932671 |
| PTP4A3 | 0.33653795 | 0.6798218 |
| RASSF3 | 0.67482527 | 0.68118708 |
| IGF2BP2 | 0.61517048 | 0.68516452 |
| TRIM59 | 0.49495112 | 0.68718385 |
| ARMCX1 | 0.67367224 | 0.6926071 |
| PDGFA | 0.69274886 | 0.64020435 |
| MAML2 | 0.69286813 | 0.61444161 |
| HOMER1 | 0.64588532 | 0.69471367 |
| SLC25A36 | 0.61822678 | 0.69515008 |
| MEX3B | 0.48825645 | 0.69525689 |
| KCNAB2 | 0.28017135 | 0.69571201 |
| RP11-4O1.2 | 0.69699882 | 0.64007133 |
| ZNF14 | 0.50911358 | 0.69700309 |
| CSGALNACT1 | 0.6972112 | 0.53962567 |
| ZNF43 | 0.69766043 | 0.66041851 |
| FAM60A | 0.69798662 | 0.57687346 |
| ZDHHC13 | 0.59284567 | 0.70134313 |
| ROR2 | 0.47953378 | 0.70146852 |
| LRRC8B | 0.70218975 | 0.43526829 |
| SNPH | 0.65861824 | 0.7030763 |
| LAPTM5 | 0.35569268 | 0.70316271 |
| PIK3IP1 | 0.45872585 | 0.70367893 |
| SIK1 | 0.57517578 | 0.70403294 |
| PDE4A | 0.50238721 | 0.70742546 |
| SLC6A8 | 0.49251283 | 0.70985862 |
| LETM2 | 0.70993884 | 0.51858834 |
| NLRP1 | 0.33686951 | 0.71202987 |
| SLFN11 | 0.57179801 | 0.71204882 |
| CDC7 | 0.71385596 | 0.46514214 |
| AFAP1 | 0.63624792 | 0.71489051 |
| ZC2HC1A | 0.62917918 | 0.71651923 |
| FMNL1 | 0.3242739 | 0.71782192 |
| OSBPL3 | 0.54660585 | 0.71872488 |
| SLC7A7 | 0.71925364 | 0.65642501 |
| NFE2L3 | 0.57475584 | 0.71968189 |
| TNFRSF21 | 0.7196947 | 0.54695308 |
| CHST10 | 0.71972882 | 0.61237824 |
| SELM | 0.56812723 | 0.71975839 |
| RP11-325F22.2 | 0.50460597 | 0.72090451 |
| MST1R | 0.47181874 | 0.7209631 |
| STX3 | 0.72102985 | 0.48367941 |
| MFSD6 | 0.52289539 | 0.72111097 |
| MIR24-2 | 0.5390643 | 0.72140079 |
| TNFRSF10A | 0.50417259 | 0.72150615 |
| GABBR1 | 0.6500145 | 0.7215756 |
| S100A4 | 0.36820521 | 0.72160432 |
| TMED3 | 0.6722162 | 0.72252257 |
| LMTK3 | 0.53581676 | 0.72422981 |
| CNN2 | 0.35223128 | 0.72444483 |
| NFATC1 | 0.53048737 | 0.72455305 |
| SGCA | 0.72589799 | 0.7198172 |
| HTR7 | 0.4759203 | 0.72789687 |
| ZNF462 | 0.73006055 | 0.4488765 |
| EPB41L4A | 0.73040779 | 0.57817447 |
| TC2N | 0.5669905 | 0.73047692 |
| STK17B | 0.45729752 | 0.73168938 |
| FAM43A | 0.38031763 | 0.7321046 |
| ZNF682 | 0.73258188 | 0.55241921 |
| CDR2L | 0.73259998 | 0.52105382 |
| ANXA1 | 0.73264023 | 0.57869759 |
| DBNDD2 | 0.73310133 | 0.73344632 |
| MCTP2 | 0.73454144 | 0.71721696 |
| CACNB1 | 0.47128632 | 0.7393145 |
| TRPC1 | 0.74188418 | 0.59654163 |
| NCR3LG1 | 0.74276061 | 0.60495753 |
| GDPD1 | 0.74610697 | 0.52045434 |
| ZNF551 | 0.5517514 | 0.74765999 |
| EFEMP1 | 0.68304411 | 0.74833159 |
| WDR54 | 0.45749948 | 0.74856452 |
| BTG2 | 0.6140358 | 0.7489323 |
| GPR160 | 0.69934449 | 0.75515911 |
| PCYOX1L | 0.49390356 | 0.75582657 |
| ENO2 | 0.56016537 | 0.75727464 |
| PTPN13 | 0.73135756 | 0.75732687 |
| MAPK10 | 0.73135737 | 0.75732839 |
| SRGAP1 | 0.7586089 | 0.6047318 |
| RP11-196G18.3 | 0.65548978 | 0.75891918 |
| METTL24 | 0.76034617 | 0.71722934 |
| LOXL1-AS1 | 0.47086974 | 0.76116581 |
| ZNF135 | 0.61234173 | 0.76129642 |
| AC006273.5 | 0.76159241 | 0.73583054 |
| SLC7A1 | 0.76343128 | 0.68634926 |
| KCNN4 | 0.39542934 | 0.76376793 |
| NRARP | 0.61413335 | 0.76444255 |
| LRRC7 | 0.76457592 | 0.65462437 |
| S100A6 | 0.71410763 | 0.76557321 |
| DUSP4 | 0.76719907 | 0.56528911 |
| TES | 0.64641779 | 0.76721734 |
| RASSF2 | 0.42520019 | 0.76744221 |
| PFKP | 0.76863343 | 0.39487891 |
| IL32 | 0.42036146 | 0.7705907 |
| C11orf63 | 0.76641642 | 0.77070393 |
| PLEKHN1 | 0.77090664 | 0.75277317 |
| SERTAD4 | 0.61386474 | 0.77235122 |
| SPHK1 | 0.77352945 | 0.59674735 |
| ZNF93 | 0.7413635 | 0.77732631 |
| DOCK8 | 0.71316828 | 0.77998158 |
| DNAJA4 | 0.41673965 | 0.78066734 |
| GUCY1A3 | 0.78101523 | 0.43909463 |
| FAT1 | 0.78161206 | 0.41320572 |
| SEZ6L2 | 0.78300846 | 0.52046814 |
| ENPP2 | 0.66985804 | 0.78327047 |
| HAGHL | 0.32832646 | 0.78364508 |
| ZNF430 | 0.62275886 | 0.78636638 |
| KIF5C | 0.56901499 | 0.78695083 |
| PMAIP1 | 0.78805908 | 0.7697196 |
| MIR155HG | 0.63833738 | 0.78900615 |
| SLC44A3 | 0.78996891 | 0.35579282 |
| OSM | 0.45717062 | 0.79018332 |
| GLIS2 | 0.58553663 | 0.79081199 |
| HS3ST1 | 0.79134804 | 0.56934978 |
| MB21D2 | 0.79162081 | 0.61279489 |
| PAPLN | 0.60978089 | 0.79186658 |
| ZNF83 | 0.74987129 | 0.79591802 |
| ZNF525 | 0.6508787 | 0.79797534 |
| JAK3 | 0.42769928 | 0.79907813 |
| CD24P4 | 0.79989556 | 0.79379019 |
| GLS | 0.76883896 | 0.80128197 |
| CDS1 | 0.80415355 | 0.63792143 |
| PDZK1IP1 | 0.80419565 | 0.59092917 |
| SGPP2 | 0.80642066 | 0.74843572 |
| EMILIN2 | 0.56552122 | 0.80927578 |
| ZNF738 | 0.71420581 | 0.8101293 |
| ZNF827 | 0.61687125 | 0.81182985 |
| KIAA1324L | 0.64072218 | 0.81313444 |
| CRIM1 | 0.81351764 | 0.5727473 |
| ARHGAP22 | 0.81355971 | 0.53217941 |
| LGALS3 | 0.81554883 | 0.80460839 |
| SLFN12 | 0.58587157 | 0.81763725 |
| AC009495.4 | 0.82025839 | 0.62715837 |
| GALNT3 | 0.8202589 | 0.62715973 |
| LAYN | 0.82570013 | 0.56557893 |
| CDH11 | 0.78947697 | 0.82706053 |
| NBEA | 0.82816261 | 0.55764296 |
| SUSD1 | 0.82864571 | 0.63820308 |
| IFI16 | 0.82900121 | 0.57093752 |
| MICAL1 | 0.40809112 | 0.83229 |
| PRDM8 | 0.4147069 | 0.83317986 |
| EPHA4 | 0.8407004 | 0.51534728 |
| IL20RA | 0.84074687 | 0.63080009 |
| MCOLN3 | 0.84799599 | 0.82310983 |
| MOXD1 | 0.61819037 | 0.85571757 |
| ZNF610 | 0.85580077 | 0.76624814 |
| C2CD4A | 0.85807699 | 0.61331564 |
| PTPN14 | 0.85928039 | 0.54240255 |
| CLDN4 | 0.86385408 | 0.70732723 |
| ZNF320 | 0.69940051 | 0.86434523 |
| SULT1C4 | 0.8666197 | 0.55853359 |
| C1orf198 | 0.86838867 | 0.75771881 |
| RP11-255H23.2 | 0.78348346 | 0.87134821 |
| RP11-54O7.17 | 0.66060703 | 0.87492254 |
| HES4 | 0.66060698 | 0.87492257 |
| ZNF439 | 0.7754003 | 0.87763927 |
| OXCT1 | 0.69052376 | 0.88093823 |
| HLA-V | 0.65968038 | 0.88135899 |
| HCG4 | 0.65968113 | 0.8813591 |
| NACAD | 0.88540406 | 0.78948956 |
| KLHL29 | 0.88688097 | 0.78426889 |
| GRAMD1B | 0.88789388 | 0.87824523 |
| BHLHE41 | 0.89380159 | 0.82046044 |
| CXCL1 | 0.89397774 | 0.49676709 |
| ETV4 | 0.89505799 | 0.77147938 |
| LOXL4 | 0.83770117 | 0.89640324 |
| SPINT1 | 0.9019053 | 0.81320729 |
| TUBB3 | 0.77901178 | 0.90258575 |
| SLFN13 | 0.71647168 | 0.90474057 |
| TESC | 0.91154566 | 0.7155896 |
| SSPN | 0.91333829 | 0.80776856 |
| HKDC1 | 0.80267098 | 0.91553292 |
| GPRC5B | 0.91582436 | 0.71925544 |
| B3GALT5 | 0.82298552 | 0.92241342 |
| LEF1 | 0.64581157 | 0.92796029 |
| CCND2 | 0.51570471 | 0.93297174 |
| BACE2 | 0.93873245 | 0.93421453 |
| LAMA2 | 0.94512492 | 0.70426537 |
| ZNF781 | 0.8958214 | 0.94739801 |
| WFDC2 | 0.95117142 | 0.84255703 |
| LPAR2 | 0.74984023 | 0.95256452 |
| ITGA2 | 0.95418097 | 0.72896514 |
| COL4A4 | 0.71663337 | 0.96095475 |
| COL4A3 | 0.71663331 | 0.96095538 |
| TAF4B | 0.60146796 | 0.96199357 |
| ITGAM | 0.9681795 | 0.8187902 |
| ASNS | 0.80819223 | 0.9686812 |
| SOX4 | 0.97014313 | 0.64336655 |
| ZNF607 | 0.96801207 | 0.97105325 |
| HSPA4L | 0.97111408 | 0.70391323 |
| PRSS22 | 0.97314734 | 0.78588991 |
| KRT80 | 0.97565683 | 0.77689948 |
| ANXA3 | 0.98288653 | 0.65418067 |
| DCDC2 | 0.98344899 | 0.84556677 |
| ZNF665 | 0.98826934 | 0.98849773 |
| C1orf106 | 0.98917225 | 0.92284703 |
| ZNF888 | 0.90202205 | 0.99204931 |
| BDKRB2 | 1.00219891 | 0.75459275 |
| ZNF66 | 0.78637292 | 1.00612414 |
| AC098614.2 | 0.73989189 | 1.0094951 |
| ANKRD18A | 1.01070424 | 0.79144155 |
| FAM201A | 1.01070424 | 0.79144103 |
| KLF5 | 1.01939278 | 0.61866171 |
| ITGB8 | 1.02049996 | 0.95067059 |
| ST8SIA1 | 0.52806315 | 1.02256245 |
| ADAMTS9-AS2 | 1.03801979 | 0.67690411 |
| ADAMTS9 | 1.0380198 | 0.67690424 |
| ZNF793 | 1.03634612 | 1.04053639 |
| C3orf52 | 1.05646192 | 0.74854065 |
| CXCR4 | 0.69277409 | 1.05694656 |
| TGFB2 | 1.07245542 | 0.66816217 |
| YBX3 | 1.07488767 | 0.60213429 |
| CXCL6 | 1.07602493 | 0.82122851 |
| F2RL1 | 1.07822298 | 0.62809111 |
| GULP1 | 1.08334898 | 0.87106295 |
| ZNF382 | 0.91514769 | 1.08943678 |
| EVC | 0.70441736 | 1.09029465 |
| TNFRSF11B | 1.09291997 | 0.80432709 |
| PDX1 | 1.03806686 | 1.10210071 |
| MDFI | 0.99970847 | 1.10729961 |
| NPNT | 1.11084697 | 0.90699949 |
| PMEPA1 | 1.11864875 | 0.98597334 |
| FAM150B | 1.12938884 | 0.93355004 |
| WNT10A | 0.54025736 | 1.13200548 |
| TTC9 | 0.91326661 | 1.1326541 |
| TMEM55A | 1.13757001 | 1.02024542 |
| EVC2 | 0.79916052 | 1.13916517 |
| HSPB8 | 1.02574973 | 1.15133921 |
| ZNF431 | 1.05242004 | 1.1614527 |
| B3GNT3 | 1.16547116 | 1.13459562 |
| GABRE | 1.16594765 | 0.83124387 |
| CTD-2008P7.9 | 1.16713333 | 0.99437014 |
| TBC1D30 | 1.18821614 | 0.75123115 |
| GABRB3 | 1.08299275 | 1.19747739 |
| RASEF | 1.22724328 | 1.07280705 |
| VCAN | 1.22905187 | 0.76885504 |
| LRRC1 | 1.16117843 | 1.23945445 |
| KRT7 | 1.29272853 | 1.01287264 |
| ZNF714 | 0.99537496 | 1.30371306 |
| CLIC6 | 1.32775759 | 1.0734942 |
| DUSP8 | 1.11637838 | 1.33563505 |
| CDH6 | 1.38128848 | 1.12788111 |
| EPCAM | 1.39394879 | 1.36241594 |
| SNAP25 | 1.47530976 | 1.0638733 |
| ID4 | 1.24692876 | 1.52077185 |
| SOX9 | 1.55632138 | 1.6100915 |
| DTNA | 1.74642847 | 1.81480646 |

TABLE S4
Prognostic epigenetic signature - 25 gene subset. The common
prognostic subset of signature genes in NASH and CHC (related
to FIG. 1). List of the 25 genes with the highest prediction
of HCC risk predicted from the 1693 commonly changed genes
on CHC and NASH patients (FDR < 0.25). The dysregulation
was determined by the nearest template prediction

| High risk genes | Low risk genes |
|---|---|
| GPRIN3 | GSTA1 |
| COL1A2 | GRB14 |
| SLC7A6 | SERPINA5 |
| CHST11 | CAT |
| LBH | SLC25A1 |
| TRPC1 | PKLR |
| IGF2BP2 | ADH4 |
| ARRDC2 | GLYAT |
| SELM | TTR |
| TMED3 | HPX |
| | RARRES2 |
| | ACADSB |
| | CFHR5 |
| | DCXR |
| | GALK1 |

### REFERENCES

1. Bray F, Ferlay J, Soerjomataram I, et al. Global cancer statistics 2018: GLOBOCAN estimates of incidence and mortality worldwide for 36 cancers in 185 countries. CA Cancer J Clin. 2018; 68(6):394-424.
2. Kanwal F, Kramer J, Asch S M, et al. Risk of Hepatocellular Cancer in HCV Patients Treated With Direct-Acting Antiviral Agents. Gastroenterology. 2017; 153(4):996-1005 e1.
3. Kim D, Li A A, Perumpail B J, et al. Changing Trends in Etiology-Based and Ethnicity-Based Annual Mortality Rates of Cirrhosis and Hepatocellular Carcinoma in the United States. Hepatology. 2019; 69(3): 1064-74.
4. Fujiwara N, Friedman S L, Goossens N, et al. Risk factors and prevention of hepatocellular carcinoma in the era of precision medicine. J Hepatol. 2017; 68(3):526-549.
5. Polak P, Karlić R, Koren A, et al. Cell-of-origin chromatin organization shapes the mutational landscape of cancer. Nature. 2015; 518(7539):360-364.
6. Hamdane N, Juhling F, Crouchet E, et al. HCV-Induced Epigenetic Changes Associated With Liver Cancer Risk Persist After Sustained Virologic Response. Gastroenterology. 2019; 156(8):2313-2329.e7.
7. Perez S, Kaspi A, Domovitz T, et al. Hepatitis C virus leaves an epigenetic signature post cure of infection by direct-acting antivirals. PLoS Genet. 2019; 15(6):e1008181.
8. Berdasco M, Esteller M. Clinical epigenetics: Seizing opportunities for translation. Nature

Reviews Genetics. 2019; 20(2):109-27.
9. Hardy T, Mann D A. Epigenetics in liver disease: From biology to therapeutics. Gut. 2016; 65(11):1895-1905.
10. Pérez-Salvia M, Esteller M. Bromodomain inhibitors and cancer therapy: From structures to applications. Epigenetics. 2016; 12(5):323-39.
11. Schmidl C, Rendeiro A F, Sheffield N C, et al. ChIPmentation: fast, robust, low-input ChIP-seq for histones and transcription factors. Nature Methods. 2015; 12(10):963-5.
12. Boldanova T, Suslov A, Heim M H, et al. Transcriptional response to hepatitis C virus infection and interferon-alpha treatment in the human liver. EMBO Molecular Medicine. 2017; 9(6):816-834.
13. Febbraio M A, Reibe S, Shalapour S, et al. Preclinical Models for Studying NASH-Driven HCC: How Useful Are They? Cell Metab. 2019; 29(1):18-26.
14. Lupberger J, Croonenborghs T, Roca Suarez A A, et al. Combined Analysis of Metabolomes, Proteomes, and Transcriptomes of Hepatitis C Virus-Infected Cells and Liver to Identify Pathways Associated With Disease Development. Gastroenterology. 2019; 157(2):537-551.e9.
15. King L Y, Canasto-Chibuque C, Johnson K B, et al. A genomic and clinical prognostic index for hepatitis C-related early-stage cirrhosis that predicts clinical deterioration. Gut. 2015; 64(8):1296-302.
16. Hoshida Y. Nearest template prediction: a single-sample-based flexible class prediction with confidence assessment. PloS One. 2010; 5(11):e15543.
17. Reich M, Liefeld T, Gould J, et al. Genepattern 2.0. Nature Genetics. 2006; 38(5):500-1.
18. Subramanian A, Tamayo P, Mootha V K, et al. Gene set enrichment analysis: a knowledge-based approach for interpreting genome-wide expression profiles. Proc Natl Acad Sci. 2005; 102(43):15545-50.
19. Nakagawa S, Wei L, Song W M, et al. Molecular Liver Cancer Prevention in Cirrhosis by Organ Transcriptome Analysis and Lysophosphatidic Acid Pathway Inhibition. Cancer Cell. 2016; 30(6):879-90.
20. Jung H R, Kang H M, Ryu J W, et al. Cell Spheroids with Enhanced Aggressiveness to Mimic Human Liver Cancer In Vitro and In Vivo. Sci Rep. 2017; 7(1):10499.
21. Song Y, Kim J S, Kim S H, et al. Patient-derived multicellular tumor spheroids towards optimized treatment for patients with hepatocellular carcinoma. J Exp Clin Cancer Res. 2018; 37(1):109.
22. Sandhu D S, Baichoo E, Roberts L R. Fibroblast growth factor signaling in liver carcinogenesis. Hepatology. 2014; 59(3):1166-73.
23. Ruan W, Pan R, Shen X, et al. cDH11 promotes liver fibrosis via activation of hepatic stellate cells. Biochem Biophys Res Commun. 2019; 508(2):543-9.
24. Lu J, Xia Y, Chen K, et al. Oncogenic role of the Notch pathway in primary liver cancer.

Oncology Letters. 2016; 12(1):3-10.
25. Tee A R, Fingar D C, Manning B D, et al. Tuberous sclerosis complex-1 and -2 gene products function together to inhibit mammalian target of rapamycin (mTOR)-mediated downstream signaling. Proc Natl Acad Sci USA. 2002; 99(21):13571-6.
26. Hoshida Y, Villanueva A, Sangiovanni A, et al. Prognostic gene expression signature for patients with hepatitis C-related early-stage cirrhosis. Gastroenterology. 2013; 144(5):1024-30.
27. Kanwal F, Kramer J R, Mapakshi S, et al. Risk of Hepatocellular Cancer in Patients With Non-Alcoholic Fatty Liver Disease. Gastroenterology. 2018; 155(6):1828-37 e2.
28. Bauhofer O, Ruggieri A, Schmid B, et al. Persistence of HCV in quiescent hepatic cells under conditions of an interferon-induced antiviral response. Gastroenterology. 2012; 143(2):429-38.e8.
29. Christen V, Treves S, Duong F H T, et al. Activation of endoplasmic reticulum stress response by hepatitis viruses up-regulates protein phosphatase 2A. Hepatology. 2007; 46(2):558-65.
30. Egnatchik R A, Leamy A K, Jacobson D A, et al. ER calcium release promotes mitochondrial dysfunction and hepatic cell lipotoxicity in response to palmitate overload. Molecular metabolism. 2014; 3(5):544-53.
31. Sehgal P, Szalai P, Olesen C, et al. Inhibition of the sarco/endoplasmic reticulum (ER) Ca′-ATPase by thapsigargin analogs induces cell death via ER Ca′ depletion and the unfolded protein response. The Journal of Biological Chemistry. 2017; 292(48):19656-73.
32. Bowers E M, Yan G, Mukherjee C, et al. Virtual ligand screening of the p300/CBP histone acetyltransferase: identification of a selective small molecule inhibitor. Chem Biol. 2010; 17(5):471-82.
33. Cabo E, Wysocka J. Modification of enhancer chromatin: what, how, and why? Mol Cell. 2013; 49(5):825-37.
34. Karatas H, Townsend E C, Cao F, et al. High-affinity, small-molecule peptidomimetic inhibitors of MLL1/WDR5 protein-protein interaction. J Am Chem Soc. 2013; 135(2):669-82.
35. Lobera M, Madauss K P, Pohlhaus D T, et al. Selective class IIa histone deacetylase inhibition via a nonchelating zinc-binding group. Nature chemical biology. 2013; 9(5):319-25.
36. Vigushin D M, Ali S, Pace P E, et al. Trichostatin A is a histone deacetylase inhibitor with potent antitumor activity against breast cancer in vivo. Clin Cancer Res. 2001; 7(4):971-6.
37. Bayo J, Fiore E J, Dominguez L M, et al. A comprehensive study of epigenetic alterations in hepatocellular carcinoma identifies potential therapeutic targets. J Hepatol. 2019; 71(1):78-90.
38. Delmore J E, Issa G C, Lemieux M E, et al. BET bromodomain inhibition as a therapeutic strategy to target c-Myc. Cell. 2011; 146(6):904-17.
39. Hong S H, Eun J W, Choi S K, et al. Epigenetic reader BRD4 inhibition as a therapeutic strategy to suppress E2F2-cell cycle regulation circuit in liver cancer. Oncotarget. 2016; 7(22):32628-40.
40. Li G Q, Guo W Z, Zhang Y, et al. Suppression of BRD4 inhibits human hepatocellular carcinoma by repressing MYC and enhancing BIM expression. Oncotarget. 2016; 7(3):2462-74.
41. Lee J S, Chu I S, Mikaelyan A, et al. Application of comparative functional genomics to identify best-fit mouse models to study human cancer. Nature Genetics. 2004; 36(12):1306-11.
42. Zanconato F, Battilana G, Forcato M, et al. Transcriptional addiction in cancer cells is mediated by YAP/TAZ through BRD4. Nature Medicine. 2018; 24(10):1599-610.
43. Nicolas G, Francesco N. Insulin resistance, non-alcoholic fatty liver disease and hepatitis C virus infection. Reviews on Recent Clinical Trials. 2014; 9(3):204-9.
44. Fujimoto A, Furuta M, Totoki Y, et al. Whole-genome mutational landscape and characterization of noncoding and structural mutations in liver cancer. Nature Genetics. 2016; 48:500-9.
45. Schulze K, Imbeaud S, Letouzé E, et al. Exome sequencing of hepatocellular carcinomas identifies new mutational signatures and potential therapeutic targets. Nature Genetics. 2015; 47(5):505-11.
46. Forner A, Reig M, Bruix J. Hepatocellular carcinoma. The Lancet. 2018; 391(10127):1301-14.
47. Villanueva A. Hepatocellular carcinoma. N Engl J Med. 2019; 380(15):1450-62.
48. Ding N, Hah N, Yu R T, et al. BRD4 is a novel therapeutic target for liver fibrosis. Proc Natl Acad Sci USA. 2015; 112(51):15713-8.
49. Stratton M S, Haldar S M, McKinsey T A. BRD4 inhibition for the treatment of pathological organ fibrosis. F1000Research. 2017; 6:F1000 Faculty Rev-15.
50. Stathis A, Bertoni F. BET Proteins as Targets for Anticancer Treatment. Cancer Discovery. 2018; 8(1):24-36.
51. Kleiner D E, Brunt E M, Van Natta M, et al. Design and validation of a histological scoring system for nonalcoholic fatty liver disease. Hepatology 2005; 41:1313-21.
52. Intraobserver and interobserver variations in liver biopsy interpretation in patients with chronic hepatitis C. The French METAVIR Cooperative Study Group. Hepatology 1994; 20:15-20.
53. Bedossa P, Poynard T. An algorithm for the grading of activity in chronic hepatitis C. The METAVIR Cooperative Study Group. Hepatology 1996; 24:289-93.
54. Verrier E R, Colpitts C C, Bach C, et al. A targeted functional RNA interference screen uncovers glypican 5 as an entry factor for hepatitis B and D viruses. Hepatology 2016; 63:35-48.
55. Yan H, Zhong G, Xu G, et al. Sodium taurocholate cotransporting polypeptide is a functional receptor for human hepatitis B and D virus. Elife 2012; 1:e00049.

1. A method of diagnosis and/or prognosis of liver disease progression, survival and/or risk of hepatocellular carcinoma in a subject comprising detecting an epigenetic or transcriptomic change in subject with liver disease, the method comprising comparing
a) the level of expression of a marker or a plurality of markers in a subject sample; and
b) the level of expression of the marker or plurality of markers in a control sample,
wherein the marker or plurality of markers are selected from the group consisting of the genes listed in table S3 and a significant difference between the level of expression of the marker or plurality of markers in the subject sample and the control sample is an indication that the subject is at risk for progression of liver disease, at risk of poor survival and/or at risk of developing a hepatocellular carcinoma.

2. The method of claim 1, wherein the liver disease is a non-alcoholic or alcoholic liver disease, a liver disease due to viral hepatitis or liver fibrosis.

3. The method of claim 2, wherein the liver disease is a non-alcoholic or alcoholic steatohepatitis, chronic hepatitis A, B, C, D or E related liver disease or liver fibrosis.

4. The method according to claim 1, wherein the subject is a patient cured by direct-acting antivirals (DAA) and/or interferon-alfa based treatment or a patient cured of or with controlled viral infection by any treatment.

5. The method according to claim 1, wherein the marker or at least one marker of the plurality of markers have increased expression in the subject sample relative to the control sample.

6. The method according to claim 1, wherein the marker or at least one marker of the plurality of markers have decreased expression in the subject sample relative to the control sample.

7. The method according to claim 1, wherein at least one marker has increased expression in the subject sample relative to the control sample and at least one marker has decreased expression in the subject sample relative to the control sample.

8. The method according to claim 1, wherein at least one gene of the high-risk gene of Table S3 is overexpressed and/or wherein at least one gene of the low-risk gene of Table S3 is underexpressed, in the subject sample in comparison to the control sample.

9. The method according to claim 1, wherein the subject has undergone tumor resection.

10. The method according to claim 1, wherein the subject sample is obtained from a non-tumorous liver tissue or a tissue surrounding a resected tumor.

11. The method according to claim 1, wherein the subject sample is selected from the group consisting of fresh tissue, fresh frozen tissue, fixed embedded tissue, patient-derived spheroids, serum, plasma or urine.

12. The method according to claim 11, wherein the patient-derived spheroids were generated by culturing fresh liver tissue in spheroid culture medium.

13. (canceled)

14. The method according to claim 1, wherein the marker is or the plurality of markers are a gene selected from the group consisting of GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

15. A method of assessing the efficacy of a therapy for liver disease and/or hepatocellular carcinoma prevention or treatment in a subject with liver disease, the method comprising comparing:
a) the level of expression of a marker or a plurality of markers in a subject sample; and
b) the level of expression of the marker or plurality of markers in a second subject sample following the treatment with the therapy,
wherein the marker or plurality of markers are selected from the group consisting of the genes listed in tables S3 and a significant difference between the level of expression of the marker or plurality of markers indicates the efficacy of the prevention or treatment of liver disease and/or hepatocellular carcinoma.

16. The method of claim 15, wherein (i) the subject is at risk for progression of liver disease, death and/or developing a hepatocellular carcinoma and/or (ii) the liver disease is a non-alcoholic or alcoholic steatohepatitis, chronic hepatitis A, B, C, D or E-related liver disease or liver fibrosis.

17. A method of identifying a compound useful for the prevention or treatment of liver disease and/or hepatocellular carcinoma, said method comprising the steps of:
a) providing a sample;
b) contacting the sample with a candidate compound; and
c) detecting an increase or decrease in expression of a marker or a plurality of markers selected from the group consisting of the genes listed in Table S3, relative to a control, and
d) identifying the compound as useful for the prevention or treatment of liver disease and/or hepatocellular carcinoma if it increases or decreases the expression of said marker or at least a marker of the plurality of markers relative to the control.

18. The method according to claim 17, wherein the genes is the subset of 25-genes presented in Table S4, and wherein the candidate compound is identified as an agent useful for agent for treatment of liver disease or prevention and treatment of hepatocellular carcinoma if the candidate compound suppresses the expression of the 10 HCC high-risk genes, or of a subset thereof and/or induces the expression of the 15 HCC low-risk genes, or of a subset thereof.

19. The method according to claim 17, wherein the sample is or comprises a subject-derived HCC or adjacent liver tissue, a cancer cell, a liver cell line, a combination of liver and non-liver cell lines including non-parenchymal cells or a cell line derived from a subject-derived HCC or adjacent liver tissue plasma, serum or urine.

20. The method according to claim 17, wherein the candidate compound is a chromatin modifier, regulator or reader inhibitor or compound targeting epigenetic gene regulation.

21. The method according to claim 20, wherein the chromatin modifier, regulator or reader inhibitor or compound targeting epigenetic gene regulation is selected from the list the group consisting of BRPF1B inhibitors, G9a/GLP inhibitors, PCAF/GCN5 inhibitors, LSD1 inhibitors, SPIN1 inhibitors, CREBBP/EP300 inhibitors, SMYD2 inhibitors, PRDM9 inhibitors, SMARCA2/4 inhibitors, EZH2 inhibitors, BAZ2A/2B inhibitors, SUV420H1/H2 inhibitors, CECR2/BPTF inhibitors, L3MBTL3 inhibitors, ATAD2A/B inhibitors or PRMT4/6 inhibitor.

22. A method for preventing or delaying the progression of a liver disease, delaying the onset of or treating hepatocellular carcinoma in a subject comprising:
performing the steps of the method of diagnosis and/or prognosis of liver disease progression and/or risk of hepatocellular carcinoma according to claim 1 or 14, and
administering a preventive treatment to the subject diagnosed as at risk for progression of liver disease and/or at risk of developing a hepatocellular carcinoma.

23. A kit for the diagnosis and/or prognosis of liver disease progression, survival and/or risk of hepatocellular carcinoma, wherein said kit comprises means for assessing the level of expression of GPRIN3, COL1A2, SLC7A6, CHST11, LBH, TRPC1, IGF2BP2, ARRDC2, SELM, TMED3, GSTA1, GRB14, SERPINA5, CAT, SLC25A1, PKLR, ADH4, GLYAT, TTR, HPX, RARRES2, ACADSB, CFHR5, DCXR and GALK1.

24. A method for generating a cellular model for liver disease or hepatocellular carcinoma (HCC) development and progression, said method comprising steps of:
(a) differentiating liver cancer cell line to obtain hepatocyte-like cells; and
(b) submitting said hepatocyte-like cells to one hepatocarcinogenic/fibrosis causing agent such as hepatitis C virus or free fatty acids to obtain liver cells exhibiting a Prognostic Epigenetic Signature (PES) high-risk gene signature

25. The method according to claim 24, wherein the liver cancer cell line is selected from the group consisting of the Huh6, Huh7, Huh7.5.1, Hep3B.1-7, HepG2, SkHepI, C3A, PLC/PRF/5 and SNU-398 cell lines or optionally a combination with another cell line such as LX2 cells or THP1 cells or another cell line or liver non-parenchymal cells such as Kupffer cells, or myofibroblasts or liver sinusoidal endothelial cells.

26. A method for identifying an agent for the treatment or prevention of liver disease and HCC, wherein said method comprises the use of a cellular model for liver disease progression and HCC risk according to claim 24.
